(19) United States
(12) Patent Application Publication
(10) Pub. No.: US 2007/0190029 A1
(43) Pub. Date: Aug. 16, 2007

(54) Listeria-induced immunorecruitment and activation, and methods of use thereof

(75) Inventors: Drew M. Pardoll, Brookeville, MD (US); Richard D. Schulick, Baltimore, MD (US); Keith S. Bahjat, Concord, CA (US); Dirk G. Brockstedt, Oakland, CA (US); Thomas W. Dubensky JR., Piedmont, CA (US); Martin A. Giedlin, Moraga, CA (US); Kiyoshi Yoshimura, Baltimore, MD (US); Ajay Jain, Baltimore, MD (US)

Correspondence Address: MORRISON & FOERSTER LLP, 755 PAGE MILL RD, PALO ALTO, CA, 94304-1018, US

(73) Assignees: Cerus Corporation, Concord, CA (US); The Johns Hopkins University, Baltimore, MD (US)

(21) Appl. No.: 11/507,734

(22) Filed: Aug. 21, 2006

Related U.S. Application Data
(60) Provisional application No. 60/709,699, filed on Aug. 19, 2005.

(51) Int. Cl. A61K 48/00; A61K 39/02; C12N 1/21

(52) U.S. Cl. 424/93.2; 424/200.1; 435/252.3

(57) ABSTRACT

Provided are reagents and methods for administering an attenuated bacterium for use in treating a cancerous or infectious condition. Reagents and methods for administering an attenuated bacterium for use in inducing an immune response against a tumor, cancer cell, or infective agent are further provided. Also provided are methods of diagnosis and kits.

### CROSS-REFERENCE TO RELATED APPLICATIONS

This application claims the priority benefit of U.S. Ser. No. 60/709,699, filed Aug. 19, 2005, the contents of which are hereby incorporated by reference herein in their entirety.

### STATEMENT REGARDING FEDERALLY SPONSORED RESEARCH OR DEVELOPMENT

This invention was made, in part, with U.S. government support under National Cancer Institute NHI 1 K23CA104160-01. The government may have certain rights in the invention.

### FIELD OF THE INVENTION

The present invention relates to compositions and methods for immunorecruitment. In particular, it provides an attenuated Listeria bacterium for treating tumors, tumor metastases, precancerous disorders, and infections.

### BACKGROUND OF THE INVENTION

Liver cancer is the fifth most common malignancy in men, and the eighth most common malignancy in women, worldwide. The disorder affects mainly persons with cirrhosis of the liver, where cirrhosis can arise from, e.g., hepatitis or alcoholism. Risk factors for liver cancer include, e.g., hepatitis B, hepatitis C, chronic exposure to dietary aflatoxin, and alcoholism. In view of the fact that hepatitis is an important risk factor, it should be noted that in the United States, about 1.2 million persons and 3.9 million persons are chronically infected with hepatitis B and C, respectively (see, e.g., (Mulhall and Younossi (2005) J. Clin. Gastroenterol. 39 (1 Suppl.):S23-S37; Bosch, et al. (2004) Gasteroenterol. 127 (5 Suppl. 1):S5-S16; Llovet, et al. (2004) Liver Transpl. 10 (2 Suppl. 1):S1 15-S120; Guyton and Kensler (2002) Curr. Oncol. Rep. 4:464-470; Kensler, et al. (2002) Eur. J. Cancer Prev. 11 Suppl. 2:S58-S64; Schiff and Ozden (2003) Alcohol Res. Health 27:232-239; Kensler, et al. (2004) Gasteroenterol. 127 (5 Suppl. 1) S310-S18; Szabo, et al. (2004) Pathol. Oncol. Res. 10:5-11; Poynard, et al. (2003) Lancet 362:2095-2100; Alter (1997) Clin Liver Dis. 1:559-68, CDC (2004) MMWR Morb. Mortal Weekly Rep. 52:1252-1254).

Liver tumors can arise by way of a primary tumor or by way of metastasis. The liver is a common site for tumor metastasis. Tumors of the liver can originate via metastasis from other parts of the liver (e.g., from hepatocytes, bile duct epithelium, endothelial cells, and the biliary tree), as well as from the stomach, colon, pituitary, pancreas, lungs, parotid, thyroid, uveal melanoma, and other tissues, such as the small intestines (see, e.g., Chen, et al. (2000) J. Hepatol. 33:91-98; Broelsch, et al. (2004) Surg. Clin. North Am. 84:495-511; Chen, et al. (1998) Hepatogastroenterol. 45:492-495; Kanoh, et al. (2004) J. Pharmacol. Exp. Therapeutics 308:168-174; Suzuki, et al. (2002) Endocr. J. 49:153-158; Matthews, et al. (2000) Am. Surg. 66:1116-1122; Cervone, et al. (2000) Am. Surg. 66:611-615; Obara, et al. (1998) Med. Oncol. 15:292-294; Olsha, et al. (1995) Invasion Metastasis 15:163-166; Martin, et al. (2003) J. Am. Coll. Surg. 196:402-409; Salvatori, et al. (2004) J. Endocrinol. Invest. 27:52-56; Feldman, et al. (2004) Ann. Surg. Oncol. 11:290-297; Kursar, et al. (2002) J. Immunol. 168:6382-6387; Nishikawa, et al. (1998) Microbiol. Immunol. 42:325-327).

Hepatocellular carcinoma is the most common form of primary liver cancer. Other liver cancers include hepatoblastoma (a cancer of children), angiosarcoma, and epithelioid hemangioendotheliioma. Related cancers include cancers of the bile duct (cholangiocarcinoma) and gallbladder (see, e.g., DeVita, et al. (eds.) (2001) Cancer of the Liver and Biliary Tree in Cancer Principles and Practice of Oncology 6^{th} ed., Lippincott, Williams, and Wilkens, Phila. PA, pp. 1162-1203; Curley (1998) Liver Cancer, M. D. Anderson Solid Tumor Oncology Series, Springer-Verlag, NY, N.Y.).

Liver cancers are usually not discovered until when they are at an advanced state and, when discovered, they are resistant to chemotherapy. Partial hepatectomy is the most common treatment, but most partial hepatectomy patients experience reoccurrences. Liver transplantation is also an effective treatment of liver cancer, but here long term survival is about the same as with partial hepatectomy. Other treatments include 5-fluorouracil, doxorubicin, tumor necrosis factor, cis-platin, and radiation (see, e.g., Ruan and Warren (2004) Surg. Oncol. Clin. N. Am. 13:505-516; Christoforidis, et al. (2002) Eur. J. Surg. Oncol. 28:875-890; Yogita and Tashiro (2000) J. Med. Invest. 47:91-100; Carr (2004) Gasteroenterol. 127 (5 Suppl. 1) S218-S224).

There has been some interest in using the Gram positive bacterium Listeria monocytogenes (L. monocytogenes) for treating experimental tumors in animals. Listeria has been administered by way of intratumoral injections (Bast, et al. (1975) J. Natl. Cancer Inst. 54:757-761). Listeria, both heat-killed or viable, administered as a mixture with an experimental tumor cell line, or injected directly into a tumor, inhibited subsequent growth of the tumor cells in vivo (see, e.g., Bast, et al. (1975) J. Natl. Cancer Inst. 54:757-761; Youdim (1976) J. Immunol. 116:579-584; Youdim (1977) Cancer Res. 37:572-577; Fulton, et al. (1979) Infection Immunity 25:708-716; Keller, et al. (1989) Int. J. Cancer 44:512-317; Keller, et al. (1990) Eur. J. Immunol. 20:695-698; Pace, et al. (1985) J. Immunol. 134:977-981). Related studies demonstrated that there was no inhibition of tumor growth where Listeria was systemically disseminated (or where the Listeria was administered at a different site from the site of the administered tumor cells) (Youdim, et al. (1974) J. Natl. Cancer Inst. 52:193-198). Mycobacterium bovis BCG has also been used to stimulate immune response, though this bacterium is unusually slow growing, and resists modification by genetic engineering or transduction.

L. monocytogenes has a natural tropism for the liver and spleen and, to some extent, other tissues such as the small intestines (see, e.g., Dussurget, et al. (2004) Ann. Rev. Microbiol. 58:587-610; Gouin, et al. (2005) Curr. Opin. Microbiol. 8:35-45; Cossart (2002) Int. J. Med. Microbiol. 291:401-409; Vazquez-Boland, et al. (2001) Clin. Microbiol. Rev. 14:584-640; Schluter, et al. (1999) Immunobiol. 201:188-195). Where the bacterium resides in the intestines, passage to the bloodstream is mediated by listerial proteins, such as actA and internalin A (see, e.g., Manohar, et al. (2001) Infection Immunity 69:3542-3549; Lecuit, et al. (2004) Proc. Natl. Acad. Sci. USA 101:6152-6157; Lecuit and Cossart (2002) Trends Mol. Med. 8:537-542). Once the bacterium enters a host cell, the life cycle of L. monocytogenes involves escape from the phagolysosome and to the cytosol. This life cycle contrasts with that of Mycobacterium, which remains inside the phagolysosome (see, e.g., Clemens, et al. (2002) Infection Immunity 70:5800-5807; Schluter, et al. (1998) Infect. Immunity 66:5930-5938; Gutierrez, et al. (2004) Cell 119:753-766). L. monocytogenes' escape from the phagolysosome is mediated by listerial proteins, such as listeriolysin (LLO), PI-PLC, and PC-PLC (see Portnoy, et al. (2002) J. Cell Biol. 158:409-414).

As both metabolically active L. monocytogenes and heat-killed L. monocytogenes have been used in studies of immune response, it should be noted that these two preparations do not stimulate the immune system in the same way. Regarding the differences between metabolically active Listeria and heat-killed Listeria, and without limiting the present invention to any mechanism, or excluding the present invention from any mechanism, it should be noted that heat-killed Listeria can produce an immune response, but where protection is not long lasting; that heat-killed Listeria can induce CD8⁺ T cells, but the CD8⁺ T cells are functionally impaired; that Listeria blocked in metabolism generally can stimulate immune response by cross-presentation, but not cross-presentation MHC Class I epitopes; that Listeria that cannot express listeriolysin (LLO) (e.g., heat-killed Listeria) fail to enter the cytoplasm and fail to efficiently induce, e.g., IL-12, MCP-1, CD40, and CD80 (see, e.g., Emoto, et al. (1997) Infection Immunity 65:5003-5009; Vazquez-Boland, et al. (2001) Clin. Microbiol. Revs. 14:584-640; Brzoza, et al. (2004) J. Immunol. 173:2641-2651; Serbina, et al. (2003) Immunity 19:891-901; Janda, et al. (2004) J. Immunol. 173:5644-5651; Kursar, et al. (2004) J. Immunol. 172:3167-3172; Brunt, et al. (1990) J. Immunol. 145:3540-3546; Lauvau, et al. (2001) Science 294:1735-1739).

Methods for treating cancers, tumors, metastases, precancerous disorders, dysplasias, and infections are often ineffective. The present invention fulfills this need by providing an attenuated Listeria for use in immunorecruitment against tumors and infections in the liver and in other tissues, e.g., for treatment of metastatic liver cancer.

### SUMMARY OF THE INVENTION

The present invention is based, in part, on the recognition that administering an attenuated Listeria monocytogenes to a mammal bearing a liver tumor resulted in enhanced survival, where the Listeria monocytogenes was not engineered to contain a nucleic acid encoding a non-listerial antigen that stimulates immune response against a tumor. The invention provides a variety of Listeria, compositions, and methods for treating cancerous or infectious conditions in a mammal, and for inducing an innate and/or an adaptive (i.e., acquired) immune response.

In some aspects, the invention provides a method for treating a mammal having a cancerous or non-listerial infectious condition, comprising administering to the mammal an effective amount of an attenuated Listeria. In some embodiments, the Listeria does not comprise a nucleic acid encoding a non-listerial antigen capable of stimulating a specific immune response against the condition (e.g., a tumor antigen or antigen from an infective agent causing the infectious condition). In some embodiments, the Listeria is administered to the mammal in the absence of a separately generated, vaccine-induced immune response to the cancerous or infectious condition in the mammal. In some embodiments, the cancerous or infectious condition is in the liver of the mammal. In some embodiments, the attenuated Listeria is metabolically active. In some embodiments, the attenuated Listeria is capable of accessing the cytosol of a cell from a phagocytic vacuole.

In some aspects, the invention provides a method for inducing an immune response against a cancer cell, tumor, or non-listerial infective agent in a mammal, comprising administering to the mammal an effective amount of an attenuated Listeria. In some embodiments, the attenuated Listeria is not administered orally to the mammal, is administered as a composition that is at least 99% free of other types of bacteria, is administered in a pharmaceutical composition, and/or is a non-naturally occurring strain. In some embodiments, the Listeria does not comprise a nucleic acid encoding a non-listerial antigen capable of stimulating a specific immune response against the cancer cell, tumor, or infective agent (e.g., a tumor antigen or antigen from the infective agent). In some embodiments, the Listeria is administered to the mammal in the absence of a separately generated, vaccine-induced immune response to the cancerous or infectious condition in the mammal. In some embodiments, the mammal comprises the cancer cell, tumor, or non-listerial infective agent in its liver. In some embodiments, the attenuated Listeria is metabolically active. In some embodiments, the attenuated Listeria is capable of accessing the cytosol of a cell from a phagocytic vacuole. In some embodiments, the immune response is an innate immune response (e.g., an NK-mediated innate immune response), an adaptive immune response (e.g., a systemic, tumor-specific memory response), or both.

In some aspects, the invention provides methods for inhibiting or reducing a cancerous disorder or condition, and/or an infectious disorder or condition.

The present invention provides a method for inhibiting or reducing a cancerous or infectious condition in a mammal having the condition, comprising administering to the mammal an effective amount of a metabolically active attenuated Listeria, wherein the Listeria does not comprise a nucleic acid encoding a non-listerial antigen capable of stimulating a specific immune response against the condition. In another embodiment, the invention provides the above method, wherein the Listeria cannot do one or more of: a. form colonies; b. replicate; or c. divide. Yet another embodiment provides the above method, wherein the metabolically active attenuated Listeria has a transcription rate that is at least: a. 10%; b. 20%; c. 50%; or d. 90%, that of a parental or wild type Listeria.

The present invention provides a method for inhibiting or reducing a cancerous or infectious condition in a mammal having the condition, comprising administering to the mammal an effective amount of an attenuated Listeria, wherein the Listeria does not comprise a nucleic acid encoding a non-listerial antigen capable of stimulating a specific immune response against the condition.

Another aspect of the present invention provides the above method, wherein the Listeria is metabolically active and cannot do one or more of: a. form colonies; b. replicate; or c. divide. Yet another aspect provides the above method, wherein the Listeria is essentially metabolically inactive. A further embodiment provides the above method, wherein the condition comprises a tumor, cancer, or pre-cancerous disorder. Yet another embodiment provides the above method, wherein the condition comprises an infection. Furthermore, what is provided is the above method wherein the infectious condition comprises one or more of: a. hepatitis B; b. hepatitis C; c. human immunodeficiency virus (HIV); d. cytomegalovirus (CMV); e. Epstein-Barr virus (EBV); or f. leishmaniasis. Also, supplied is the above method that inhibits or reduces one, or any combination, of the: a. number of tumors or cancer cells; b. tumor mass; or c. titer of an infectious agent, in the mammal. In addition, the present invention embraces the above method wherein the condition is of the liver. Moreover, the invention embraces the above method wherein the attenuated Listeria comprises a recombinant nucleic acid encoding one or more of: a. an antibiotic resistance gene; b. a mutated actA gene; or c. a mutated inlB gene. In yet another aspect, the present invention contemplates the above method, wherein the attenuated Listeria is attenuated in one or more of: a. growth; b. cell-to-cell spread; c. binding to or entry into a host cell; d. replication; or e. DNA repair. What is supplied by the invention is the above method, wherein the Listeria is attenuated by one or more of: a. an actA mutation; b. an inlB mutation; c. a uvrA mutation; d. a uvrB mutation; e. a uvrC mutation; f. a nucleic acid targeting compound; or g. a uvrAB mutation and a nucleic acid targeting compound. What is also encompassed, is the above method wherein the nucleic acid targeting compound is a psoralen. Also encompassed is the above method, wherein the administering stimulates an innate immune response. Yet another embodiment is the above method, wherein the administering stimulates an acquired immune response. And another embodiment is the above method, wherein the administering stimulates one, or any combination, of a: a. NK cell; b. NKT cell; c. dendritic cell (DC); d. monocyte or macrophage; e. neutrophil; or f. toll-like receptor (TLR) or nucleotide-binding oligomerization domain (NOD) protein, as compared with immune response in the absence of the administering of the effective amount of the attenuated Listeria.

Embraced by the present invention, is the above method, wherein the administering stimulates increased expression of any one, or any combination, of: a. CD69; b. interferon-gamma (IFNgamma); c. interferon-alpha (IFNalpha) or interferon-beta (IFNbeta); d. interleukin-12 (IL-12), monocyte chemoattractant protein (MCP-1), or e. interleukin-6 (IL-6), as compared with expression in the absence of the administering of the effective amount of the attenuated Listeria. Also embraced, is the above method, wherein the administering of the attenuated Listeria is one, or any combination, of: a. intravenous; b. intramuscular; c. subcutaneous; or d. oral. What is also supplied, is the above method, wherein the mammal is human. Moreover, what is supplied is the above method, wherein the Listeria is Listeria monocytogenes. Furthermore, what is supplied is the above method, further comprising administering one, or any combination of: a. an agonist or antagonist of a cytokine; b. an inhibitor of a T regulatory cell (Treg); or c. a tumor cell attenuated in growth or replication. In yet a further aspect, what is provided is the above method, wherein the inhibitor of a Treg is cyclophosphamide (CTX). The present invention also encompasses the above method, wherein the mammal comprises hepatic leukocytes, and the administering stimulates one or both of: a. an increase in the percent of hepatic leukocytes that is NK cells, compared to the percent without the administering of the attenuated Listeria; or b. an increase in expression of an activation marker by a hepatic NK cell, compared to the expression without the administering of the attenuated Listeria. Moreover, what is provided is the above method, wherein the increase in the percent of hepatic leukocytes that is NK cells is at least: a. 5%; b. 10%; c. 15%; d. 20%; or e. 25%, greater than compared to the percent without the administering of the attenuated Listeria. Also encompassed, is the above method, wherein the attenuated Listeria is one or both of: a. not administered orally to the mammal, or b. administered as a composition that is at least 99% free of other types of bacteria.

In some aspects, the invention provides methods for enhancing survival.

What is provided is a method for enhancing survival to a cancerous or infectious condition in a mammal having the condition, comprising administering to the mammal an effective amount of an attenuated Listeria, wherein the Listeria does not comprise a nucleic acid encoding a non-listerial antigen capable of stimulating a specific immune response against the condition. Also provided is the above method, wherein the Listeria is metabolically active and cannot do one or more of: a. form colonies; b. replicate; or c. divide. Yet another aspect provides the above method wherein the Listeria is essentially metabolically inactive.

In another embodiment, the present invention provides a method for enhancing survival to a cancerous or infectious condition in a mammal having the condition, comprising administering to the mammal an effective amount of a metabolically active attenuated Listeria, wherein the Listeria does not comprise a nucleic acid encoding a non-listerial antigen capable of stimulating a specific immune response against the condition. Yet another embodiment provides the above method, wherein the metabolically active attenuated Listeria has a transcription rate that is at least: a. 10%; b. 20%; c. 50%; or d. 90%, that of a parental or wild type Listeria.

Yet another embodiment provides the above method, wherein the survival time is enhanced as compared to survival with an appropriate control mammal not administered the attenuated Listeria. Moreover, what is embraced by the present invention is the above method, wherein the survival time is enhanced by at least: a. five days; b. ten days; c. fifteen days; or d. twenty days, as compared to survival with an appropriate control mammal not administered the attenuated Listeria. Supplied is the above method, wherein the condition comprises a cancer, tumor, or pre-cancerous disorder. Also supplied, is the above method, wherein the condition comprises an infection. Moreover, in another aspect, the present invention provides the above method, wherein the infectious condition comprises one or more of: a. hepatitis B; b. hepatitis C; c. human immunodeficiency virus (HIV); d. cytomegalovirus (CMV); e. Epstein-Barr virus (EBV); or f. leishmaniasis.

Additionally, what is supplied is the above method for enhancing survival, wherein the condition is of the liver. Furthermore, what is supplied is the above method, wherein the attenuated Listeria comprises a recombinant nucleic acid encoding: a. an antibiotic resistance gene; b. a mutated actA gene; or c. a mutated inlB gene. In yet a further aspect, what is supplied by the present invention, is the above method wherein the attenuated Listeria is attenuated in one or more of: a. growth; b. cell-to-cell spread; c. binding to or entry into a host cell; d. replication; or e. DNA repair. What is also embraced by the present invention, is the above method, wherein the Listeria is attenuated by one or more of: a. an actA mutation; b. an inlB mutation; c. a uvrA mutation; d. a uvrB mutation; e. a uvrC mutation; f. a nucleic acid targeting compound; or g. a uvrAB mutation and a nucleic acid targeting compound. Moreover, what is embraced is the above method, wherein the nucleic acid targeting compound is a psoralen. In yet another aspect, the present invention provides the above method, wherein the administering stimulates an innate immune response. Also, what is supplied is the above method, wherein the administering stimulates an acquired immune response. Moreover, provided is the above method, wherein the administering stimulates one, or any combination, of a: a. NK cell; b. NKT cell; c. dendritic cell (DC); d. monocyte or macrophage; e. neutrophil; f. toll-like receptor (TLR); or g. nucleotide-binding oligomerization domain protein (NOD protein). Additionally, what is provided is the above method, wherein the administering stimulates increased expression of any one, or any combination, of: a. CD69; b. interferon-gamma (IFNgamma); c. interferon-alpha (IFNalpha) or interferon-beta (IFNbeta); d. interleukin-12 (IL-12); e. monocyte chemoattractant protein (MCP-1); or f. interleukin-6 (IL-6), as compared with expression in the absence of the administering of the effective amount of the attenuated Listeria. Furthermore, an additional embodiment that is provided by the present invention, is the above method, wherein the administering of the attenuated Listeria is one, or any combination, of: a. intravenous; b. intramuscular; c. subcutaneous; or d. oral. Also supplied, is the above method wherein the mammal is human. Moreover, supplied is the above method, wherein the Listeria is Listeria monocytogenes. Additionally, what is embraced by the present invention, is the above method, further comprising administering one, or any combination of: a. an agonist or antagonist of a cytokine; b. an inhibitor of a T regulatory cell (Treg); or c. a tumor cell attenuated in growth or replication. Yet another aspect, is the above method, wherein the inhibitor of a Treg is cyclophosphamide (CTX). Further, another aspect is the above method, wherein the mammal comprises hepatic leukocytes, and the administering stimulates one or both of: a. an increase in the percent of hepatic leukocytes that is NK cells, compared to the percent without the administering of the attenuated Listeria; or b. an increase in expression of an activation marker by a hepatic NK cell, compared to the expression without the administering of the attenuated Listeria. Also embraced by the present invention, is the above method, wherein the increase in the percent of hepatic leukocytes that is NK cells is at least: a. 5%; b. 10%; c. 15%; d. 20%; or e. 25%, greater than compared to the percent without the administering of the attenuated Listeria. Supplied by the invention is the above method, wherein the administered attenuated Listeria is one or both of: a. not administered orally to the mammal; or b. administered as a composition that is at least 99% free of other types of bacteria.

In another aspect, the present invention provides a method for inhibiting or reducing a cancerous or infectious condition in a mammal having the condition, comprising administering to the mammal an effective amount of an attenuated Listeria, wherein the attenuation is in one or more of the: a. actA gene; b. inlB gene; c. uvrA gene; d. uvrB gene; or e. uvrC gene, and wherein the Listeria does not comprise a nucleic acid encoding a non-listerial antigen capable of stimulating a specific immune response against the disorder.

Yet another aspect of the present invention provides a method for enhancing survival to a cancerous or infectious condition in a mammal having the condition, comprising administering to the mammal an effective amount of an attenuated Listeria, wherein the attenuation is in one or more of: a. an actA gene; b. an inlB gene; c. a uvrA gene; d. a uvrB gene; or a uvrC gene, and wherein the Listeria does not comprise a nucleic acid encoding a non-listerial antigen capable of stimulating a specific immune response against the disorder.

In some embodiments, the methods (and reagents) disclosed above encompass using an attenuated Listeria that comprises a nucleic acid encoding at least one tumor antigen, an attenuated Listeria that comprises a nucleic acid encoding at least one cancer antigen, an attenuated Listeria that comprises a nucleic acid encoding at least one heterologous antigen, or an attenuated Listeria that expresses at least one tumor antigen, cancer antigen, and/or heterologous antigen.

In some embodiments, the methods (and reagents) disclosed above encompass using an attenuated Listeria that does not comprise a nucleic acid encoding a tumor antigen, an attenuated Listeria that does not comprise a nucleic acid encoding a cancer antigen, an attenuated Listeria that does not comprise a nucleic acid encoding a heterologous antigen, or an attenuated Listeria that does not express a tumor antigen, cancer antigen, and/or a heterologous antigen.

In some embodiments, the methods (and reagents) disclosed above encompass using an attenuated Listeria that comprises a nucleic acid encoding an antigen from a non-listerial infectious organism. In some embodiments, the methods (and reagents) disclosed above encompass using an attenuated Listeria that does comprise a nucleic acid encoding an antigen from a virus or a parasite.

In some embodiments, the methods (and reagents) disclosed above encompass using an attenuated Listeria that does not comprise a nucleic acid encoding an antigen from a non-listerial infectious organism. In some embodiments, the methods (and reagents) disclosed above encompass using an attenuated Listeria that does not comprise a nucleic acid encoding an antigen from a virus or a parasite.

In some embodiments of each of the aforementioned methods, as well as other methods described herein, the methods do not encompass administering an additional vaccine to the mammal against the cancerous or infectious condition (or against the cancer cell, tumor, or infectious agent). In some embodiments of each of the aforementioned methods, as well as other methods described herein, a vaccine against the cancerous or infectious condition (or against the cancer cell, tumor, or infectious agent) has not previously been administered to the mammal. In some embodiments of each of the aforementioned methods, as well as other methods described herein, the Listeria is administered to the mammal in the absence of a separately generated, vaccine-induced immune response to the cancerous or infectious condition (or to the cancer cell, tumor, or infective agent) in the mammal.

In some embodiments of each of the aforementioned methods, as well as other methods described herein, the infectious condition or infective agent is non-listerial. In some embodiments of each of the aforementioned methods, as well as other methods described herein, the Listeria is administered in multiple doses. In some embodiments of each of the aforementioned methods, as well as other methods described herein, the Listeria is not attenuated with HIV-gag. In some embodiments of each of the aforementioned methods, as well as other methods described herein, the attenuated Listeria is capable of accessing the cytosol of a cell from a phagocytic vacuole.

In some embodiments of each of the above-disclosed methods, the attenuated Listeria is not prepared by growing on a medium based on animal protein, but is prepared by growing on a different type of medium. In some embodiments of each of the above-disclosed methods, the attenuated Listeria is not prepared by growing on a medium containing peptides derived from animal protein, but is prepared by growing on a different type of medium. Moreover, in some embodiments of each of the above-disclosed methods, the attenuated Listeria is administered by a route that is not oral or that is not enteral. Additionally, in some embodiments of each of the above-disclosed methods, the attenuated Listeria is administered by a route that does not require movement from the gut lumen to the lymphatics or bloodstream.

In some embodiments of each of the above-disclosed methods, the Listeria are not injected directly into the tumor or are not directly injected into a site that is affected by the cancerous or infectious disorder.

Additionally, each of the above embodiments encompasses administering the Listeria by direct injection into a tumor, by direct injection into a cancerous lesion, and/or by direct injection into a lesion of infection. Also, the invention includes each of the above embodiments, where administration is not by direct injection into a tumor, not by direct injection into a cancerous lesion, and/or not by direct injection into a lesion of infection.

Provided is a vaccine where the heterologous antigen, as in any of the embodiments disclosed herein, is a tumor antigen or is derived from a tumor antigen. Also provided is a vaccine where the heterologous antigen, as in any of the embodiments disclosed herein, is a cancer antigen, or is derived from a cancer antigen. Moreover, what is provided is a vaccine where the heterologous antigen, as in any of the embodiments disclosed herein, is an antigen of an infectious organism, or is derived from an antigen of an infectious organism, e.g., a virus, bacterium, parasite, or multi-cellular organism.

A further embodiment provides a nucleic acid where the heterologous antigen, as in any of the embodiments disclosed herein, is a tumor antigen or derived from a tumor antigen. Also provided is a nucleic acid where the heterologous antigen, as in any of the embodiments disclosed herein, is a cancer antigen, or is derived from a cancer antigen. Moreover, what is provided is a nucleic acid, where the heterologous antigen, as in any of the embodiments disclosed herein, is an antigen of an infectious organism, or is derived from an antigen of an infectious organism, e.g., a virus, bacterium, parasite, or multi-cellular organism.

In another embodiment, what is provided is a Listeria where the heterologous antigen, as in any of the embodiments disclosed herein, is a tumor antigen or derived from a tumor antigen. Also provided is a Listeria where the heterologous antigen, as in any of the embodiments disclosed herein, is a cancer antigen, or is derived from a cancer antigen. Moreover, what is provided is a Listeria, where the heterologous antigen, as in any of the embodiments disclosed herein, is an antigen of an infectious organism, or is derived from an antigen of an infectious organism, e.g., a virus, bacterium, parasite, or multi-cellular organism.

In some embodiments, each of the methods disclosed above encompasses an attenuated Listeria that is not prepared by growing on a medium based on animal or meat protein, but is prepared by growing on a different type of medium. Provided is an attenuated Listeria not prepared by growing on a medium based on meat or animal protein, but is prepared by growing on a medium based on yeast and/or vegetable derived protein.

In some embodiments, the invention provides a method for treating a mammal having a cancerous or non-listerial infectious condition, wherein the cancerous or infection condition is in the liver of the mammal, wherein the method comprises administering to the mammal an effective amount of a metabolically active, attenuated Listeria, wherein the Listeria does not comprise a nucleic acid encoding a non-listerial antigen capable of stimulating a specific immune response against the condition, and wherein the attenuated Listeria is administered to the mammal in multiple doses. In some embodiments, the mammal has the cancerous condition (e.g., a condition comprising a tumor and/or cancer). In some embodiments, the mammal has the non-listerial infectious condition (e.g., a condition comprising an infection). The invention encompasses methods of treatment in which the cancerous or infectious condition is inhibited or reduced in the mammal by the administration of the effective amount of the attenuated Listeria. The invention further encompasses methods of treatment in which the survival of the mammal is enhanced by the administration of the effective amount of the attenuated Listeria. In some embodiments, the attenuated Listeria is attenuated in one or more of growth, cell to cell spread, binding to or entry into a host cell, replication, or DNA repair. In some embodiments, the Listeria is attenuated by one or more of an actA mutation, an inlB mutation, a uvrA mutation, a uvrB mutation, a uvrC mutation, a nucleic acid targeting compound, or a uvrAB mutation and a nucleic acid targeting compound. In some embodiments, the Listeria cannot do one or more of form colonies, replicate, or divide. In some embodiments, the attenuated Listeria is administered intravenously. In some embodiments, the attenuated Listeria is administered in three or more doses. In some embodiments, the attenuated Listeria is not administered orally to the mammal, is not administered as a composition that is at least 99% free of other types of bacteria, is administered to the mammal in a pharmaceutical composition, and/or is not naturally occurring. In some embodiments, the mammal has not previously been administered a vaccine against the cancerous or infectious condition. In some embodiments, the method does not further comprise administering an additional vaccine against the cancerous or infectious condition to the mammal. The administering of the Listeria may stimulate an innate immune response and/or an acquired immune response. In some embodiments, the mammal is human. In some embodiments, the Listeria is Listeria monocytogenes. In some embodiments, the effective amount comprises at least about 1×10³ CFU/kg or at least about 1×10³ Listeria cells/kg.

The invention further provides a method for inducing an immune response against a cancer cell, tumor, or non-listerial infective agent in a mammal (e.g., human), wherein the mammal comprises the cancer cell, tumor, or non-listerial infective agent in its liver, wherein the method comprises administering to the mammal an effective amount of a metabolically active, attenuated Listeria, wherein the Listeria does not comprise a nucleic acid encoding a non-listerial antigen capable of stimulating a specific immune response against the condition, wherein the attenuated Listeria is administered to the mammal in multiple doses. In some embodiments, the Listeria is not administered orally to the mammal, is administered as a composition that is at least 99% free of other types of bacteria, is administered in a pharmaceutical composition, and/or is not a non-naturally occurring strain. In some embodiments, the attenuated Listeria is attenuated in one or more of growth, cell to cell spread, binding to or entry into a host cell, replication, or DNA repair. In some embodiments, the Listeria is attenuated by one or more of an actA mutation, an inlB mutation, a uvrA mutation, a uvrB mutation, a uvrC mutation, a nucleic acid targeting compound, or a uvrAB mutation and a nucleic acid targeting compound. In some embodiments, the Listeria cannot form colonies, replicate, and/or divide. In some embodiments, the attenuated Listeria is administered intravenously. In some embodiments, the attenuated Listeria is administered in three or more doses. The administering of the Listeria may stimulate an innate immune response and/or an acquired immune response. In some embodiments, the Listeria are a strain of Listeria monocytogenes. In some embodiments, the effective amount comprises at least about 1×10³ CFU/kg or at least about 1×10³ Listeria cells/kg. In some embodiments, the method does not further comprise administering an additional vaccine capable of stimulating a specific immune response against the cancer cell, tumor, or non-listerial infective agent to the mammal. In some embodiments the mammal comprises the cancer cell or tumor. In some embodiments, the mammal comprises the infective agent.

In some embodiments, the invention provides a method for treating a mammal having a cancerous or non-listerial infectious condition, wherein the cancerous or infectious condition is in the liver of the mammal, comprising administering to the mammal an effective amount of a metabolically active, attenuated Listeria, wherein the Listeria does not comprise a nucleic acid encoding a non-listerial antigen capable of stimulating a specific immune response against the condition. In some embodiments, the Listeria is administered to the mammal in the absence of a separately generated, vaccine-induced immune response to the cancerous or infectious condition in the mammal. In some embodiments, the attenuated Listeria is capable of accessing the cytosol of a cell from a pliagocytic vacuole. In some embodiments, the attenuated Listeria is attenuated in one or more of growth, cell to cell spread, binding to or entry into a host cell, replication, or DNA repair. In some embodiments, the Listeria is attenuated by one or more of an actA mutation, an inlB mutation, a uvrA mutation, a uvrB mutation, a uvrC mutation, a nucleic acid targeting compound, or a uvrAB mutation and a nucleic acid targeting compound. In some embodiments, the Listeria cannot do one or more of form colonies, replicate, or divide. In some embodiments, the attenuated Listeria is administered intravenously. In some embodiments, the attenuated Listeria is administered in multiple doses (e.g., three or more doses). In some embodiments, the attenuated Listeria is not administered orally to the mammal, is not administered as a composition that is at least 99% free of other types of bacteria, is administered to the mammal in a pharmaceutical composition, and/or is not naturally occurring. In some embodiments, the mammal has not previously been administered a vaccine against the cancerous or infectious condition. In some embodiments, the method does not further comprise administering an additional vaccine against the cancerous or infectious condition to the mammal. The administering of the Listeria may stimulate an innate immune response and/or an acquired immune response. In some embodiments, the mammal is human. In some embodiments, the Listeria is Listeria monocytogenes. In some embodiments, the effective amount comprises at least about 1×10³ CFU/kg or at least about 1×10³ Listeria cells/kg.

In certain embodiments, the invention provides a method for inducing an immune response against a cancer cell, tumor, or non-listerial infective agent in a mammal, wherein the mammal comprises the cancer cell, tumor, or non-listerial infective agent in its liver, comprising administering to the mammal an effective amount of a metabolically active, attenuated Listeria, wherein the Listeria does not comprise a nucleic acid encoding a non-listerial antigen capable of stimulating a specific immune response against the condition, and wherein the attenuated Listeria is not administered orally to the mammal, is administered as a composition that is at least 99% free of other types of bacteria, is administered in a pharmaceutical composition, and/or is a non-naturally occurring strain. In some embodiments, the Listeria is administered to the mammal in the absence of a separately generated, vaccine-induced immune response to the cancer cell, tumor, or infective agent in the mammal. In some embodiments, the attenuated Listeria is capable of accessing the cytosol of a cell from a phagocytic vacuole. In some embodiments, the immune response is an innate immune response (e.g., an NK-mediated innate immune response), an acquired immune response (e.g., a systemic, tumor-specific memory response), or both. In some embodiments, the attenuated Listeria is attenuated in one or more of growth, cell to cell spread, binding to or entry into a host cell, replication, or DNA repair. In some embodiments, the Listeria is attenuated by one or more of an actA mutation, an inlB mutation, a uvrA mutation, a uvrB mutation, a uvrC mutation, a nucleic acid targeting compound, or a uvrAB mutation and a nucleic acid targeting compound. In some embodiments, the Listeria cannot form colonies, replicate, and/or divide. In some embodiments, the attenuated Listeria is administered intravenously. In some embodiments, the attenuated Listeria is administered in multiple (e.g., three or more doses). In some embodiments, the Listeria is a strain of Listeria monocytogenes. In some embodiments, the effective amount comprises at least about 1×10³ CFU/kg or at least about 1×10³ Listeria cells/kg. In some embodiments, the method does not further comprise administering an additional vaccine capable of stimulating a specific immune response against the cancer cell, tumor, or non-listerial infective agent to the mammal.

In some embodiments of each of the aforementioned methods, as well as other methods described herein, the attenuated Listeria is an actA deletion mutant or an actAinlB double deletion mutant.

The invention further provides compositions, such as vaccine, immunogenic compositions, and pharmaceutical compositions, comprising each of the aforementioned Listeria, as well as other Listeria and reagents described herein (e.g., in the Detailed Description or Examples below). The use of each of the Listeria described herein in the manufacture of a pharmaceutical composition or medicament is likewise provided. The pharmaceutical compositions or medicaments may be used in any of the methods described herein. For example, the invention provides the use of each of the Listeria described herein in the manufacture of a medicament for the treatment of a cancerous condition or (non-listerial) infectious condition in a mammal. The invention further provides the use of each of the Listeria described herein in the manufacture of a medicament for inducing an immune response against a cancer cell, tumor, or non-listerial infective agent in a mammal.

Further descriptions of the aspects and embodiments described above, as well as additional embodiments and aspects of the invention, are provided below.

### BRIEF DESCRIPTION OF THE DRAWINGS

FIGS. 1A to 1E disclose survival data.

FIG. 1A demonstrates that administering L. monocytogenes ΔactA or L. monocytogenes ΔactAΔinlB improved survival to tumors, where the bacteria were not engineered to express any heterologous antigen. This figure shows the survival in response to different numbers of doses, that is, one dose, three doses, or three doses.

FIG. 1B also demonstrates that administering L. monocytogenes ΔactA or L. monocytogenes ΔactAΔinlB increased survival to tumors, where the bacteria were not engineered to express any heterologous antigen. This figure shows the survival in response to different numbers of doses, that is, doses at intervals of three days, or at intervals of one week.

FIG. 1C reveals that L. monocytogenes ΔactAΔinlB increased survival to tumors, where the bacteria were not engineered to express any heterologous antigen. Doses were provided at intervals of three days, and here one of three different levels of bacteria were administered. Also, doses were provided at weekly intervals, and here again, one of three different levels of bacteria was given.

FIG. 1D demonstrates that administering CTX (at t=4 days) alone results in some increase in survival, and that administering CTX (at t=4 days) plus Listeria (Listeria administered at days 5, 12, and 19; Listeria administered at days 6, 13, and 20; or Listeria at days 7, 14, and 21) results in even greater survival.

FIG. 1E discloses the results of progressively delaying combination therapy with CTX plus Listeria ΔactAΔinlB.

FIG. 1F reveals survival of mice to CT26 tumors, where CT26 tumor cell inoculated mice were treated with Lm ΔactAΔinlB or with no Lm ΔactAΔinlB, as indicated. Mice also received no antibody, or antibodies that specifically deplete CD4⁺ T cells; CD8⁺ T cells; or NK cells, as indicated.

FIG. 1G reveals survival of mice to CT26 tumors, where CT26-tumor cell inoculated mice were treated with Listeria ΔactA plus GM CSF vaccine (GVAX), along with agents that specifically deplete CD4⁺ T cells, CD8⁺ T cells, or NK cells.

FIG. 1H shows the percentage of mice that were tumor free at 60 days after tumor re-challenge. Results are shown for control mice (“Control”) and long term survivors that were previously injected with Lm ΔactAΔinlB following inoculation with CT26. The long term survivors were re-challenged without injection of depleting antibodies (“No antibody”), following injection of anti-CD4⁺ antibodies (“Anti-CD4⁺ antibody”), or following injection of anti-CD8⁺ antibodies (“Anti-CD8⁺ antibody”).

FIG. 2A demonstrates that administering attenuated Listeria resulted in a dose-dependent increase in hepatic NK cells.

FIG. 2B shows that administering attenuated Listeria did not increase the percent of splenic NK cells.

FIG. 2C reveals that administering attenuated Listeria increased expression of CD69 by hepatic NK cells in a dose dependent manner.

FIG. 2D reveals that administering attenuated Listeria increased expression of CD69 by splenic NK cells.

FIG. 3A discloses that administering attenuated Listeria resulted in an increase in hepatic NKT cells.

FIG. 3B discloses that administering attenuated Listeria did not increase the percent of splenic NKT cells.

FIG. 3C demonstrates that administering attenuated Listeria increased the expression of CD69 by hepatic NKT cells.

FIG. 3D demonstrates that administering attenuated Listeria increased the expression of CD69 by splenic NKT cells.

FIGS. 4A and B show that administering attenuated Listeria did not result in an increase in total T cells, as a percent of leukocytes, in the liver or spleen.

FIGS. 4C and D disclose that administering attenuated Listeria did not result in an increase in CD4⁺ T cells, as a percent of leukocytes, in the liver or spleen.

FIG. 4E demonstrates that administering attenuated Listeria stimulated the dose-dependent expression of CD69 by hepatic CD4⁺ T cells.

FIG. 4F demonstrates that administering attenuated Listeria stimulated expression of CD69 by splenic CD4⁺ T cells.

FIGS. 5A and B show that administering attenuated Listeria did not result in an increase in CD8⁺ T cells, as a percent of leukocytes, in the liver or spleen.

FIG. 5C demonstrates that administering attenuated Listeria increased CD69 expression by hepatic CD8⁺ T cells.

FIG. 5D demonstrates that administering attenuated Listeria increased CD69 expression by splenic CD8⁺ T cells.

FIG. 6A reveals that administering attenuated Listeria increased the percent of total hepatic leukocytes occurring as GR-1⁺ neutrophils.

FIG. 6B reveals that administering attenuated Listeria increased the percent of total splenic leukocytes occurring as GR-1⁺ neutrophils.

FIG. 7A indicates that administering attenuated Listeria increased the percent of hepatic CD4⁺ T cells expressing CD25.

FIG. 7B shows that administering attenuated Listeria increased the median expression of CD25 by hepatic CD4⁺ T cells.

FIG. 7C indicates that administering attenuated Listeria had little or no influence on the percent of splenic CD4⁺ T cells expressing CD25.

FIG. 7D shows that administering attenuated Listeria had little or no influence on expression of CD25 by spleen CD4⁺ T cells.

FIGS. 8 and 9 disclose time course studies.

FIG. 8A shows that administering attenuated Listeria increased the percent of hepatic leukocytes that are NK cells.

FIG. 8B shows that administering attenuated Listeria had little or no influence on the percent of splenic leukocytes that are NK cells.

FIG. 9A shows that administering attenuated Listeria increased the percent of hepatic leukocytes that are neutrophils.

FIG. 9B shows that administering attenuated Listeria increased the percent of splenic leukocytes that are neutrophils.

FIGS. 10 to 13 disclose results with administration of a vaccine comprising an attenuated tumor cell engineered to express a cytokine (GM-CSF). This vaccine is called GVAX. The term “GVAX,” “GM vaccine,” and “GM-CSF vaccine” may be used interchangeably.

FIGS. 10A, 10B, 10C, 10D, 10E, 10F, 10G, 10H, and 10I disclose the immune responses in the liver following administration of L. monocytogenes ΔactA (the Listeria was not modified to contain a nucleic acid encoding a heterologous antigen.) Also shown are immune responses in the liver following administration of both the Listeria and the GVAX vaccine. The immune responses followed include NK cell number (FIG. 10A); NKT cell number (FIG. 10B); CD8⁺ T cell number (FIG. 10C); plasmacytoid DC number (FIG. 10D); myeloid DC number (FIG. 10E); tumor specific CD8⁺ T cell number (FIG. 10F); as well as cell activation as assessed by expression of mRNA encoding interferon-gamma (FIGS. 10G and 10H). FIG. 10I shows FACS analysis of CD8⁺ T cells from liver of CT26 tumor cell-treated mice, where mice had also been administered with, e.g., various therapeutic agents.

FIGS. 11A and B demonstrate that administering the vaccine alone resulted in some increase in survival, while administering an attenuated Listeria with the vaccine produced greater survival. The number of bacteria administered was 10⁷ colony forming units (1e7 colony forming units; CFU).

FIG. 12 demonstrates that giving the vaccine (GM) alone resulted in a slight improvement in survival, while giving vaccine plus an attenuated Listeria (GM+Lm actA or GM+Lm actA/inlB) resulted in greater survival, while giving the GM vaccine plus an attenuated Listeria and cyclophosphamide (CTX), resulted in even greater survival.

FIGS. 13A to C demonstrate survival to tumors, where animals were administered with the vaccine (GM) only, or vaccine (GM) plus different levels of an attenuated L. monocytogenes.

FIG. 13A shows survival data with L. monocytogenes ΔactA (deletion mutant) administered at 3×10⁶ CFU, 1×10⁷ CFU, or 3×10⁷ CFU.

FIG. 13B discloses survival data with L. monocytogenes ΔactAΔinlB (deletion mutant) administered at 3×10⁶ CFU, 1×10⁷ CFU, or 3×10⁷ CFU.

FIG. 13C reveals survival data with the vaccine only, or with L. monocytogenes ΔactAΔinlB administered at 3×10³ CFU, 3×10⁴ CFU, 3×10⁵ CFU, 3×10⁶ CFU, or 3×10⁷ CFU.

FIG. 14 discloses treatment of lung tumors with L. monocytogenes ΔactAΔinlB.

FIG. 15 shows memory response (Elispot assays) resulting from a re-challenge with CT26 tumor cells, where tumor-inoculated mice had initially been treated with no therapeutic agent, Listeria only, GM-CSF vaccine plus Listeria, or cyclophosphamide (CTX) only.

FIG. 16 shows tumor volume of tumors resulting from a re-challenge with CT26 tumor cells, where tumor-inoculated mice had initially been treated with no therapeutic agent, Listeria only, GM-CSF vaccine plus Listeria, or cyclophosphamide (CTX) only.

FIG. 17 shows cytokine expression.

FIG. 18 discloses NK cell activation and recruitment, and MCP-1 expression.

FIG. 19A discloses expression of IL-1 Ralpha in monkeys, after administering Lm ΔactAΔinlB.

FIG. 19B discloses expression of interferon-gamma (IFNgamma) in monkeys, after administering Lm ΔactAΔinlB.

FIG. 19C reveals expression of tumor necrosis factor-alpha (TNFalpha) in monkeys, after administering Lm ΔactAΔinlB.

FIG. 19D discloses expression of MCP-1 in monkeys, after administering Lm ΔactAΔinlB.

FIG. 19E demonstrates expression of MIP-1beta in monkeys, after administering Lm ΔactAΔinlB.

FIG. 19F discloses expression of interleukin-6 (IL-6) in monkeys, after administering Lm ΔactAΔinlB.

FIG. 19G discloses expression of various cytokines in monkeys, following administration of Lm ΔactAΔinlB.

FIG. 20 shows a comparison of the anti-tumor activity induced by Lm ΔactAΔinlB, heat-killed (HK) Lm ΔactAΔinlB, and Δhly Lm.

### DETAILED DESCRIPTION

As used herein, including the appended claims, the singular forms of words such as “a,” “an,” and “the” include their corresponding plural references unless the context clearly dictates otherwise. All references cited herein are incorporated by reference to the same extent as if each individual publication, sequences accessed by a GenBank Accession No., patent application, patent, Sequence Listing, nucleotide or oligo- or polypeptide sequence in the Sequence Listing, as well as figures and drawings in said publications and patent documents, was specifically and individually indicated to be incorporated by reference.

I. Definitions.

Abbreviations are often used herein to indicate a mutation in a gene, or in a bacterium encoding a gene. By way of example, the abbreviation “Listeria ΔactA,” “Lm ΔactA,” “ΔactA,”“Lm actA,” “Lm-actA,” or “Listeria actA” means that part, or all, of the actA gene is deleted. The abbreviation “Listeria ΔactAΔinlB,” “Lm ΔactAΔinlB,” “ΔactAΔinlB,” “Lm actAinlB,”“actAinlB,” “Lm actA/inlB,” “Lm-actAinlB,” or “Listeria actAinlB” means that part, or all, of both the actA gene and the inlB is deleted. Lm means “Listeria monocytogenes.” The delta symbol (Δ) means deletion. An abbreviation including a superscripted minus sign (Listeria actA⁻) means that the actA gene was mutated, e.g., by way of a deletion, point mutation, or frameshift mutation, but not limited to these types of mutations. The term “GM-CSF vaccine” is used interchangeably herein with the terms “GM vaccine” and “GVAX.” Exponentials are abbreviated. For example “3e7” means 3×10⁷.

“Administration” and “treatment,” as it applies to a human, mammal, mammalian subject, animal, veterinary subject, placebo subject, research subject, experimental subject, cell, tissue, organ, or biological fluid, refers without limitation to contact of an exogenous ligand, reagent, placebo, small molecule, pharmaceutical agent, therapeutic agent, diagnostic agent, or composition to the subject, cell, tissue, organ, or biological fluid, and the like. “Administration” and “treatment” can refer, e.g., to therapeutic, pharmacokinetic, diagnostic, research, placebo, and experimental methods. Treatment of a cell encompasses contact of a reagent to the cell, as well as contact of a reagent to a fluid, where the fluid is in contact with the cell. “Administration” and “treatment” also encompass in vitro and ex vivo treatments, e.g., of a cell, by a reagent, diagnostic, binding composition, or by another cell. Depending on the context, “treatment” of a subject can imply that the subject is in need of treatment, e.g., in the situation where the subject comprises a disorder expected to be ameliorated by administration of a reagent. The success or outcome of a treatment can be assessed by, for example, increased survival time (e.g., to a life threatening proliferative disorder), decrease in tumor size, decrease in tumor number, decrease in metastasis from a specific tissue, decrease in metastasis to a specific tissue, titer of an infectious agent, and the like, as compared with a placebo treatment or with no treatment.

An agonist, as it relates to a ligand and receptor, comprises a molecule, combination of molecules, a complex, or a combination of reagents, that stimulates the receptor. For example, an agonist of granulocyte-macrophage colony stimulating factor (GM-CSF) can encompass GM-CSF, a mutein or derivative of GM-CSF, a peptide mimetic of GM-CSF, a small molecule that mimics the biological function of GM-CSF, or an antibody that stimulates GM-CSF receptor. An antagonist, as it relates to a ligand and receptor, comprises a molecule, combination of molecules, or a complex, that antagonizes the receptor. “Antagonist” encompasses any reagent that inhibits a constitutive activity of the receptor. A constitutive activity is one that is manifest in the absence of a ligand/receptor interaction. “Antagonist” also encompasses any reagent that inhibits or prevents a stimulated (or regulated) activity of the receptor. By way of example, an antagonist of GM-CSF receptor includes, without implying any limitation, an antibody that binds to GM-CSF and prevents GM-CSF from binding to GM-CSF receptor, or an antibody that binds to GM-CSF receptor and prevents GM-CSF from binding to the receptor, or where the antibody locks the receptor in an inactive conformation.

“Antigen presenting cells” (APCs) are cells of the immune system used for presenting antigen to T cells. APCs include dendritic cells, monocytes, macrophages, marginal zone Kupffer cells, microglia, Langerhans cells, T cells, and B cells (see, e.g., Rodriguez-Pinto and Moreno (2005) Eur. J. Immunol. 35:1097-1105). Dendritic cells occur in at least two lineages. The first lineage encompasses pre-DC1, myeloid DC1, and mature DC1. The second lineage encompasses CD34⁺⁺CD45RA⁻ early progenitor multipotent cells, CD34⁺⁺CD45RA⁺ cells, CD34⁺⁺CD45RA⁺⁺CD4⁺ IL-3Ralpha⁺⁺ pro-DC2 cells, CD4⁺ CD11c⁻plasmacytoid pre-DC2 cells, lymphoid human DC2 plasmacytoid-derived DC2s, and mature DC2s (see, e.g., Gilliet and Liu (2002) J. Exp. Med. 195:695-704; Bauer, et al. (2001) J. Immunol. 166:5000-5007; Arpinati, et al. (2000) Blood 95:2484-2490; Kadowaki, et al. (2001) J. Exp. Med. 194:863-869; Liu (2002) Human Immunology 63:1067-1071).

“Attenuation” and “attenuated” encompasses a bacterium, virus, parasite, prion, tumor cell, and the like, that is modified to reduce toxicity or pathogenicity to a host. The host can be a human or animal host, or an organ, tissue, or cell. The bacterium, to give a non-limiting example, can be attenuated to reduce binding to a host cell, to reduce spread from one host cell to another host cell, to reduce extracellular growth, or to reduce intracellular growth in a host cell. Attenuation can be assessed by measuring, e.g., an indicum or indicia of toxicity, the LD₅₀, the rate of clearance from an organ, or the competitive index (see, e.g., Auerbuch, et al. (2001) Infect. Immunity 69:5953-5957). Generally, an attenuation results an increase in the LD₅₀ and/or an increase in the rate of clearance by at least 25%; more generally by at least 50%; most generally by at least 100% (2-fold); normally by at least 5-fold; more normally by at least 10-fold; most normally by at least 50-fold; often by at least 100-fold; more often by at least 500-fold; and most often by at least 1000-fold; usually by at least 5000-fold; more usually by at least 10,000-fold; and most usually by at least 50,000-fold; and conventionally by at least 100,000-fold. As non-limiting examples: a modification of a bacterium that reduces growth reduces the pathological properties of a bacterium. Thus, this modification is an attenuation. A modification of a bacterium that reduces DNA repair can reduce the pathological properties of a bacterium. Therefore, this modification is also an attenuation.

“Attenuated gene” encompasses a gene that mediates toxicity, pathology, or virulence, to a host, growth within the host, or survival within the host, where the gene is mutated in a way that mitigates, reduces, or eliminates the toxicity, pathology, or virulence. The reduction or elimination can be assessed by comparing the virulence or toxicity mediated by the mutated gene with that mediated by the non-mutated (or parent) gene. “Mutated gene” encompasses deletions, point mutations, and frameshift mutations in regulatory regions of the gene, coding regions of the gene, non-coding regions of the gene, or any combination thereof.

Attenuation can be effected by, e.g., heat-treatment or chemical modification. Attenuation can also be effected by genetic modification of a nucleic acid that modulates, e.g., metabolism, extracellular growth, or intracellular growth, genetic modification of a nucleic acid encoding a virulence factor, such as listerial prfA, actA, listeriolysin (LLO), an adhesion mediating factor (e.g., an internalin), mpl, phosphatidylcholine phospholipase C (PC-PLC), phosphatidylinositol-specific phospholipase C (PI-PLC; plcA gene), any combination of the above, and the like. Attenuation can be assessed by comparing a biological function of an attenuated Listeria with the corresponding biological function shown by an appropriate parent Listeria.

The present invention provides a Listeria that is attenuated by treating with a nucleic acid targeting agent or a nucleic acid targeted compound, such as a cross-linking agent, a psoralen, a nitrogen mustard, cis-platin, a bulky adduct, ultraviolet light, gamma irradiation, any combination thereof, and the like. The Listeria can also be attenuated by mutating at least one nucleic acid repair gene, e.g., uvrA, uvrB, uvrAB, uvrC, uvrD, uvrAB, phrA, and/or recA. Moreover, the invention provides a Listeria attenuated by both a nucleic acid targeting agent and by a mutation in a nucleic acid repair gene. Additionally, the invention encompasses treating with a light sensitive nucleic acid targeting agent, such as a psoralen, or a light sensitive nucleic acid cross-linking agent, such as psoralen, followed by exposure to ultraviolet light (see, e.g., U.S. Pat. Publication Nos. U.S. 2004/0228877 of Dubensky, et al. and U.S. 2004/0197343 of Dubensky, et al.).

“Cancerous condition” and “cancerous disorder” encompass, without implying any limitation, a cancer, a tumor, a metastasis, an angiogenesis of a tumor, and precancerous disorders such as dysplasias.

“Effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition.

An “extracellular fluid” encompasses, e.g., serum, plasma, blood, interstitial fluid, cerebrospinal fluid, secreted fluids, lymph, bile, sweat, and urine. An “extracellular fluid” can comprise a colloid or a suspension, e.g., whole blood or coagulated blood.

“Growth” of a Listeria bacterium encompasses, without limitation, functions of bacterial physiology and bacterial nucleic acids relating to colonization, replication, increase in listerial protein content, increase in listerial lipid content. Unless specified otherwise explicitly or by context, growth of a Listeria encompasses growth of the bacterium outside a host cell, and also growth inside a host cell. Growth related genes include, without implying any limitation, those that mediate energy production (e.g., glycolysis), nutrient transport, transcription, translation, and replication.

In some embodiments, “growth” refers to bacterial growth and multiplication in the cytoplasm of an infected host cell and does not refer to in vitro growth. For example, in some embodiments, a gene that is highly specific for “growth” is one which encodes a protein that does not contribute to growth in vitro, and does not contribute to growth in conventional bacterial broth, but does contribute to some extent or to a large extent to intracellular growth and multiplication in the cytoplasm of the infected host cell.

Conventionally, growth of the attenuated Listeria of the present invention is at most 80% that of the parent Listeria strain, more conventionally growth of the attenuated Listeria is at most 70% that of the parent Listeria strain, most conventionally growth of the attenuated Listeria is at most 60% that of the parent Listeria strain, normally, growth of the attenuated Listeria of the present invention is at most 50% that of the parent Listeria strain; more normally growth is at most 45% that of the parent strain; most normally growth is 40% that of the parent strain; often growth is at most 35% that of the parent strain, more often growth is at most 30% that of the parent strain; and most often growth is at most 25% that of the parent strain; usually growth is at most 20% that of the parent strain; more usually growth is at most 15% that of the parent strain; most usually growth is at most 10% that of the parent strain; typically growth is at most 5% that of the parent strain; more typically growth of the attenuated Listeria of the present invention is at most 1% that of the parent strain; and most typically growth is not detectable. Growth of the parent and the attenuated strain can be compared by measuring extracellular growth of both organisms. Growth of the parent and the attenuated strain can also be compared by measuring intracellular growth of both organisms.

A growth related gene embraces one that stimulates the rate of intracellular growth by the same amount that it stimulates the rate of extracellular growth, by at least 20% greater than it stimulates the rate of extracellular growth; more normally by at least 30% greater than the rate it stimulates extracellular growth; most normally at least 40% greater than the rate it stimulates extracellular growth; usually at least 60% greater than the rate it stimulates extracellular growth; more usually at least 80% greater than the rate it stimulates extracellular growth; most usually it stimulates the rate of intracellular growth by at least 100% (2-fold) greater than the rate it stimulates extracellular growth; often at least 3-fold greater than the rate it stimulates extracellular growth; more often at least 4-fold greater than the rate it stimulates extracellular growth; and most often at least 10-fold greater than the rate it stimulates extracellular growth; typically at least 50-fold greater than the rate it stimulates extracellular growth; and most typically at least 100-fold greater than the rate it stimulates extracellular growth.

“Immune condition” or “immune disorder” encompasses a disorder, condition, syndrome, or disease resulting from ineffective, inappropriate, or pathological response of the immune system, e.g., to a persistent infection or to a persistent cancer (see, e.g., Jacobson, et al. (1997) Clin. Immunol. Immunopathol. 84:223-243). “Immune condition” or “immune disorder” encompasses, e.g., pathological inflammation, an inflammatory disorder, and an autoimmune disorder or disease. “Immune condition” or “immune disorder” also can refer to infections, persistent infections, and proliferative conditions, such as cancer, tumors, and angiogenesis, including infections, tumors, and cancers that resist irradication by the immune system. “Immune condition” or “immune disorder” also encompasses cancers induced by an infective agent, including the non-limiting examples of cancers induced by hepatitis B virus, hepatitis C virus, simian virus 40 (SV40), Epstein-Barr virus, papillomaviruses, polyomaviruses, Kaposi's sarcoma herpesvirus, human T-cell leukemia virus, and Helicobacter pylori (see, e.g., Young and Rickinson (2004) Nat. Rev. Cancer 4:757-768; Pagano, et al. (2004) Semin. Cancer Biol. 14:453-471; Li, et al. (2005) Cell Res. 15:262-271).

“Innate immunity,” “innate response,” and “innate immune response” encompasses, without limitation, a response resulting from recognition of a pathogen-associated molecular pattern (PAMP). “Innate response” can encompass a response mediated by a toll-like receptor (TLR), mediated by a NOD protein (nucleotide-binding oligomerization domain protein), or mediated by scavenger receptors, mannose receptors, or beta-glucan receptors (see, e.g., Pashine, et al. (2005) Nat. Med. Suppl. 11:S63-S68). “Innate response” is characterized by the fact that a TLR can be stimulated by any member of a family of ligands (not merely by one ligand having a distinct structure). Moreover, “innate response” is distinguished in that a ligand that stimulates a TLR can promote a response against an antigen, where the ligand need not have any structural identity or structural similarity to the antigen. Innate response also encompasses physiological activities mediated by opsons or lectins (see, e.g., Doherty and Arditi (2004) J. Clin. Invest. 114:1699-1703; Tvinnereim, et al. (2004) J. Immunol. 173:1994-2002; Vankayalapati, et al. (2004) J. Immunol. 172:130-137; Kelly, et al. (2002) Nat. Immunol. 3:83-90; Alvarez-Dominguez, et al. (1993) Infection Immunity 61:3664-3672; Alvarez-Dominguez, et al. (2000) Immunology 101:83-89; Roos, et al. (2004) Eur. J. Immunol. 34:2589-2598; Takeda and Akira (2005) International Immunity 17:1-14; Weiss, et al. (2004) J. Immunol. 172:4463-4469; Chamaillard, et al. (2003) Cell Microbiol. 5:581-592; Philpott and Girardin (2004) Mol. Immunol. 41:1099-1108).

A composition that is “labeled” is detectable, either directly or indirectly, by spectroscopic, photochemical, biochemical, immunochemical, isotopic, or chemical methods. For example, useful labels include ³²P, ³³P, ³⁵S, ¹⁴C, ³H, ¹²⁵I, stable isotopes, epitope tags, fluorescent dyes, electron-dense reagents, substrates, or enzymes, e.g., as used in enzyme-linked immunoassays, or fluorettes (see, e.g., Rozinov and Nolan (1998) Chem. Biol. 5:713-728).

“Ligand” refers to a small molecule, peptide, polypeptide, or membrane associated or membrane-bound molecule, that is an agonist or antagonist of a receptor. “Ligand” also encompasses a binding agent that is not an agonist or antagonist, and has no agonist or antagonist properties. By convention, where a ligand is membrane-bound on a first cell, the receptor usually occurs on a second cell. The second cell may have the same identity, or it may have a different identity, as the first cell. A ligand or receptor may be entirely intracellular, that is, it may reside in the cytosol, nucleus, or in some other intracellular compartment. The ligand or receptor may change its location, e.g., from an intracellular compartment to the outer face of the plasma membrane. The complex of a ligand and receptor is termed a “ligand receptor complex.” Where a ligand and receptor are involved in a signaling pathway, the ligand occurs at an upstream position and the receptor occurs at a downstream position of the signaling pathway.

A bacterium that is “metabolically active” encompasses a bacterium, including a L. monocytogenes, where colony formation is impaired or substantially prevented but where transcription is essentially not impaired; where replication is impaired or substantially prevented but where transcription is essentially not impaired; or where cell division is impaired or substantially prevented but where transcription is essentially not impaired. A bacterium that is “metabolically active” also encompasses a bacterium, including a L. monocytogenes, where colony formation, replication, and/or cell division, is impaired or substantially prevented but where an indication of metabolism, e.g., translation, respiration, fermentation, glycolysis, motility is essentially not impaired. Various indicia of metabolism for L. monocytogenes are disclosed (see, e.g., Karlin, et al. (2004) Proc. Natl. Acad. Sci. USA 101:6182-6187; Gilbreth, et al. (2004) Curr. Microbiol. 49:95-98).

The metabolically active bacterium of the present invention encompasses a bacterium in which the level of metabolic activity as compared to that of a suitable parent (or control) bacterium, is normally at least 20% that of the parent, more normally at least 30% that of the parent, most normally at least 40% that of the parent, typically at least 50% that of the parent, more typically at least 60% that of the parent, most typically at least 70% that of the parent, usually at least 80% that of the parent, more usually at least 90% that of the parent, and most usually indistinguishable from that of the parent bacterium, and in another aspect, greater than that of the parent. In some embodiments, metabolic activity is measured in terms of total expression level or by the expression levels of one or more individual proteins. In some embodiments, expression levels are measured at the RNA level, e.g., by quantification, directly or indirectly, of RNA transcripts. In some embodiments, the expression levels are measured at the protein level, e.g., by measuring the level of protein synthesis generally.

The metabolically active bacterium of the present invention encompasses a bacterium where colony formation, replication, and/or cell division, is under 5% that of a suitable parent (or control) bacterium but where metabolism as compared to that of a suitable parent (or control) bacterium, is normally at least 20% that of the parent, more normally at least 30% that of the parent, most normally at least 40% that of the parent, typically at least 50% that of the parent, more typically at least 60% that of the parent, most typically at least 70% that of the parent, usually at least 80% that of the parent, more usually at least 90% that of the parent, and most usually indistinguishable from that of the parent bacterium, and in another aspect, greater than that of the parent.

The metabolically active bacterium of the present invention encompasses a bacterium where colony formation, replication, and/or cell division, is under 0.5% that of a suitable parent (or control) bacterium and where metabolism, as compared to that of a suitable parent (or control) bacterium, is normally at least 20% that of the parent, more normally at least 30% that of the parent, most normally at least 40% that of the parent, typically at least 50% that of the parent, more typically at least 60% that of the parent, most typically at least 70% that of the parent, usually at least 80% that of the parent, more usually at least 90% that of the parent, and most usually indistinguishable from that of the parent bacterium, and in another aspect, greater than that of the parent. Colony formation, replication, and/or cell division is measured under conditions that facilitate replication (e.g., not frozen). A bacterium that is essentially metabolically inactive includes, without limitation, a bacterium that is heat-killed. Residual metabolic activity of an essentially metabolically inactive bacterium can be due to, e.g., oxidation of lipids, oxidation of sulfhydryls, reactions catalyzed by heavy metals, or to enzymes that are stable to heat-treatment.

The metabolically active Listeria of the invention encompass Listeria having a transcription rate that is at least 10%, at least 20%, at least 50%, or at least 90% that of a parental or wild-type Listeria.

Methods of assaying the level of metabolic activity in bacteria are well known in the art. Known assay methods include, but are not limited to, S³⁵-methionine pulse chase assays of protein synthesis (e.g., see U.S. Patent Pub. No. 2004/0197343, incorporated by reference herein). Alternatively, cell viability and metabolic activity may be measured by MTT assays (e.g., see U.S. Patent Pub. No. 2004/0197343).

“Nucleic acid” refers to deoxyribonucleotides or ribonucleotides and polymers thereof in either single stranded, double-stranded form, or multi-stranded form. The term nucleic acid may be used interchangeably with gene, cDNA, mRNA, oligonucleotide, and polynucleotide, depending on the context. A particular nucleic acid sequence can also implicitly encompasses “allelic variants” and “splice variants.”

“Peptide” refers to a short sequence of amino acids, where the amino acids are connected to each other by peptide bonds. A peptide may occur free or bound to another moiety, such as a macromolecule, lipid, oligo- or polysaccharide, and/or a polypeptide. Where a peptide is incorporated into a polypeptide chain, the term “peptide” may still be used to refer specifically to the short sequence of amino acids. A “peptide” may be connected to another moiety by way of a peptide bond or some other type of linkage. A peptide is at least two amino acids in length and generally less than about 25 amino acids in length, where the maximal length is a function of custom or context. The terms “peptide” and “oligopeptide” may be used interchangeably.

“Protein” generally refers to the sequence of amino acids comprising a polypeptide chain. Protein may also refer to a three dimensional structure of the polypeptide. “Denatured protein” refers to a partially denatured polypeptide, having some residual three dimensional structure or, alternatively, to an essentially random three dimensional structure, i.e., totally denatured. The invention encompasses methods using polypeptide variants, e.g., involving glycosylation, phosphorylation, sulfation, disulfide bond formation, deamidation, isomerization, cleavage points in signal or leader sequence processing, covalent and non-covalently bound cofactors, oxidized variants, and the like. The formation of disulfide linked proteins is described (see, e.g., Woycechowsky and Raines (2000) Curr. Opin. Chem. Biol. 4:533-539; Creighton, et al. (1995) Trends Biotechnol. 13:18-23).

“Precancerous condition” encompasses, without limitation, dysplasias, preneoplastic nodules; macroregenerative nodules (MRN); low-grade dysplastic nodules (LG-DN); high-grade dysplastic nodules (HG-DN); biliary epithelial dysplasia; foci of altered hepatocytes (FAH); nodules of altered hepatocytes (NAH); chromosomal imbalances; aberrant activation of telomerase; re-expression of the catalytic subunit of telomerase; expression of endothelial cell markers such as CD31, CD34, and BNH9 (see, e.g., Terracciano and Tornillo (2003) Pathologica 95:71-82; Su and Bannasch (2003) Toxicol. Pathol. 31:126-133; Rocken and Carl-McGrath (2001) Dig. Dis. 19:269-278; Kotoula, et al. (2002) Liver 22:57-69; Frachon, et al. (2001) J. Hepatol. 34:850-857; Shimonishi, et al. (2000) J. Hepatobiliary Pancreat. Surg. 7:542-550; Nakanuma, et al. (2003) J. Hepatobiliary Pancreat. Surg. 10:265-281). Methods for diagnosing cancer and dysplasia are disclosed (see, e.g., Riegler (1996) Semin. Gastrointest. Dis. 7:74-87; Benvegnu, et al. (1992) Liver 12:80-83; Giannini, et al. (1987) Hepatogastroenterol. 34:95-97; Anthony (1976) Cancer Res. 36:2579-2583).

“Recombinant” when used with reference, e.g., to a nucleic acid, cell, animal, virus, plasmid, vector, or the like, indicates modification by the introduction of an exogenous, non-native nucleic acid, alteration of a native nucleic acid, or by derivation in whole or in part from a recombinant nucleic acid, cell, virus, plasmid, or vector. Recombinant protein refers to a protein derived, e.g., from a recombinant nucleic acid, virus, plasmid, vector, or the like. “Recombinant bacterium” encompasses a bacterium where the genome is engineered by recombinant methods, e.g., by way of a mutation, deletion, insertion, and/or a rearrangement. “Recombinant bacterium” also encompasses a bacterium modified to include a recombinant extra-genomic nucleic acid, e.g., a plasmid or a second chromosome.

“Sample” refers to a sample from a human, animal, placebo, or research sample, e.g., a cell, tissue, organ, fluid, gas, aerosol, slurry, colloid, or coagulated material. The “sample” may be tested in vivo, e.g., without removal from the human or animal, or it may be tested in vitro. The sample may be tested after processing, e.g., by histological methods. “Sample” also refers, e.g., to a cell comprising a fluid or tissue sample or a cell separated from a fluid or tissue sample. “Sample” may also refer to a cell, tissue, organ, or fluid that is freshly taken from a human or animal, or to a cell, tissue, organ, or fluid that is processed or stored.

“Specifically” or “selectively” binds, when referring to a ligand/receptor, nucleic acid/complementary nucleic acid, antibody/antigen, or other binding pair (e.g., a cytokine to a cytokine receptor) indicates a binding reaction which is determinative of the presence of the protein in a heterogeneous population of proteins and other biologics. Thus, under designated conditions, a specified ligand binds to a particular receptor and does not bind in a significant amount to other proteins present in the sample. Specific binding can also mean, e.g., that the binding compound, nucleic acid ligand, antibody, or binding composition derived from the antigen-binding site of an antibody, of the contemplated method binds to its target with an affinity that is often at least 25% greater, more often at least 50% greater, most often at least 100% (2-fold) greater, normally at least ten times greater, more normally at least 20-times greater, and most normally at least 100-times greater than the affinity with any other binding compound.

In a preferred embodiment an antibody will have an affinity which is greater than about 10⁹ liters/mol, as determined, e.g., by Scatchard analysis (Munsen, et al. (1980) Analyt. Biochem. 107:220-239). It is recognized by the skilled artisan that some binding compounds can specifically bind to more than one target, e.g., an antibody specifically binds to its antigen as well as to an Fc receptor.

“Spread” of a bacterium encompasses “cell to cell spread,” that is, transmission of the bacterium from a first host cell to a second host cell, as mediated, for example, by a vesicle. Functions relating to spread include, but are not limited to, e.g., formation of an actin tail, formation of a pseudopod-like extension, and formation of a double-membraned vacuole.

Normally, spread of an attenuated Listeria of the present invention is at most 90% that of the parent Listeria strain; more normally spread is at most 80% that of the parent strain; most normally spread is at most 70% that of the parent strain; often spread is at most 60% that of the parent strain; more often spread is at most 50% that of the parent strain; and most often spread is at most 40% that of the parent strain; usually spread is at most 30% that of the parent strain; more usually spread is at most 20% that of the parent strain; most usually spread is at most 10% that of the parent strain; conventionally spread is at most 5% that of the parent strain; more conventionally spread of the attenuated Listeria of the present invention is at most 1% that of the parent strain; and most conventionally spread is not detectable.

“Therapeutically effective amount” is defined as an amount of a reagent or pharmaceutical composition that is sufficient to show a patient benefit, i.e., to cause a decrease, prevention, or amelioration of the symptoms of the condition being treated. When the agent or pharmaceutical composition comprises a diagnostic agent, a “diagnostically effective amount” is defined as an amount that is sufficient to produce a signal, image, or other diagnostic parameter. Effective amounts of the pharmaceutical formulation will vary according to factors such as the degree of susceptibility of the individual, the age, gender, and weight of the individual, and idiosyncratic responses of the individual (see, e.g., U.S. Pat. No. 5,888,530 issued to Netti, et al.)

“Vaccine” encompasses preventative vaccines. Vaccine also encompasses therapeutic vaccines, e.g., a vaccine administered to a mammal that comprises a condition or disorder associated with the antigen or epitope provided by the vaccine.

II. General.

The present invention provides, in some aspects, reagents and methods of administering a Listeria, e.g., Listeria monocytogenes, or other listerial species, for the treatment or prevention of a condition, such as a cancerous condition and/or an infectious condition, in a mammal. In some embodiments, the condition is of the liver, or of any other organ or tissue for which Listeria has a tropism. In some embodiments, reagents and methods of administering a Listeria, e.g., Listeria monocytogenes, or other listerial species, for the treatment or prevention of an immune disorder of the liver, or of any other organ or tissue for which Listeria has a tropism are provided. Provided are reagents and methods for treating tumors, cancers, precancerous conditions, infections, and infectious disorders. The Listeria of the present invention serves as a general immunorecruiting agent, resulting in increased inflammation or in immune cell activation at one or more sites where the Listeria accumulates. As the Listeria need not be engineered to express a heterologous antigen (e.g., a tumor antigen), any one embodiment of the present invention can stimulate immune response to (or against) a plurality of tumor types (each tumor type expressing a different antigenic profile), not merely to one tumor type.

Provided are methods and reagents for treating metastasis to the liver from another tissue, e.g., from the colon to the liver, as well as for treating metastasis from the liver to another tissue (see, e.g., Yasui and Shimizu (2005) Int. J. Clin. Oncol. 10:86-96; Rashidi, et al. (2000) Clin. Cancer Res. 6:2464-2468; Stoeltzing, et al. (2003) Ann. Surg. Oncol. 10:722-733; Amemiya, et al. (2002) Ophthalmic Epidemiol. 9:35-47).

The present invention can treat liver tumors arising from de novo tumorigenesis in the liver, or from metastases to the liver from another part of the liver (e.g., from hepatocytes, bile duct epithelium, endothelial cells, and the biliary tree), or from metastasis to the liver from the gasterointestinal tract, colon, rectum, ovary, nervous system, endocrine tissues, neuroendocrine tissues, breast, lung, or other part of the body (see, e.g., Liu, et al. (2003) World J. Gastroenterol. 9:193-200; Cormio, et al. (2003) Int. J. Gynecol. Cancer 13:125-129; Sarmiento and Que (2003) Surg. Oncol. Clin. N. Am. 12:231-242; Athanbasakis, et al. (2003) Eur. J. Gastroenterol. Hepatol. 15:1235-1240; Diaz, et al. (2004) Breast 13:254-258). In some embodiments, the tumor in the liver has metastasized from the stomach, colon, pituitary, pancreas, lungs, parotid, thyroid, uveal melanoma or the small intestines. In some embodiments, the tumor in the liver is metastatic colorectal cancer. In some embodiments, the tumor is metastatic esophageal cancer.

In some embodiments, the tumor in the liver treated by the methods of the invention is a primary liver tumor. The liver cancer may, in some embodiments, be hepatocellular carcinoma, hepatoblastoma, angiosarcoma, or epithelioid hemangioendothelioma.

In some further embodiments, the cancer is a cancer of the bile duct (cholangiocarcinoma) or gallbladder.

In some embodiments, the methods of the invention do not comprise administering to the mammal both the attenuated Listeria and an additional vaccine against the condition in the mammal being treated or against a cancer cell, tumor, or infectious agent in the mammal. In some embodiments, the Listeria is administered to the mammal in the absence of a separately generated, vaccine-induced immune response to the cancer cell, tumor, or infective agent in the mammal. In some embodiments, a vaccine has not previously been administered to the mammal against the cancerous or infectious condition. In some embodiments, the vaccine which has not previously been administered to the mammal or which is not administered to the mammal as part of the methods described herein is a tumor vaccine, such as the GM-CSF vaccine described in U.S. Patent Publication No. 2006/0051380, incorporated by reference herein in its entirety. In some embodiments, the mammal has not been previously administered a vaccine that is an attenuated tumor cell line expressing GM-CSF. In some embodiments, the Listeria is not administered to the mammal as an admixture with an antigen (e.g., tumor antigen or antigen from an infectious agent).

The pathways of immune response parallel each other in mice and humans. Immune response to L. monocytogenes involves an innate response, as well as an adaptive response. Innate response is usually identified with increased activity of neutrophils, NK cells, NKT cells, DCs, monocyte/macrophages, and toll-like receptors (TLRs). The pathways of innate response largely parallel each other in mice and humans. The pathways of adaptive immunity also generally parallel each other in mice and humans. In short, innate response to Listeria involves early recruitment of cells such as neutrophils, NK cells, and monocytes, in the mouse and human. Activity of a TLR can be assessed, e.g., by measuring activity of IL-1-R associated kinase (IRAK), NF-kappaB, JNK, caspase-1 dependent cleavage of IL-18 precursor, or activation of IRF-3 (see, e.g., Takeda, et al. (2003) Ann. Rev. Immunol. 21:335-376).

Mouse and human NK cells occur as two subsets, one subset high in expression of IL-12 receptor subunit (IL-12Rbeta2) and one low in this receptor subunit. The following narrative concerns inhibitory receptors expressed by NK cells. Mouse NK cells express gp49B, similar to KIR of human NK cells. Mouse NK cells express Ly-49A, which is similar to CD94/NKG2A on human NK cells. The following concerns activating receptors on NK cells. Both mouse and human NK cells express NKG2D (see, e.g., Chakir, et al. (2000) J. Immunol. 165:4985-4993; Smith, et al. (2000) J. Exp. Med. 191:1341-1354; Ehrlich, et al. (2005) J. Immunol. 174:1922-1931; Peritt, et al. (1998) J. Immunol. 161:5821-5824).

NKT cells occur in both humans and mice. NKT cells of humans and mice show the same reactivity against glyceramides. Human and murine NKT cells express TLRs and show phenotypic and functional similarities. NKT cells mediate immune response to tumors, where IL-12 produced by a DC acts on an NKT cell, stimulating the NKT cell to produce IFNgamma which, in turn, activates NK cells and CD8⁺ T cells to kill tumors (see, e.g., Couedel, et al. (1998) Eur. J. Immunol. 28:4391-4397; Sakamoto, et al. (1999) J. Allergy Clin. Immunol. 103:S445-S451; Saikh, et al. (2003) J. Infect. Dis. 188:1562-1570). NKT cells play a role in response to Listeria (see, e.g., Emoto, et al. (1997) Infection Immunity 65:5003-5009; Taniguchi, et al. (2003) Annu. Rev. Immunol. 21:483-513; Sidobre, et al. (2004) Proc. Natl. Acad. Sci. 101:12254-12259).

In both the mouse and humans, monocytes serve as precursors to macrophages and dendritic cells. The CX₃CR1^{low} monocytes of mice correspond to the CD14^{high}CD16⁻monocytes of humans. The CX₃CR1^{high} monocytes of mice correspond to CD14^{low}CD16^{high} of humans (Sunderkotter, et al. (2004) J. Immunol. 172:4410-4417).

Both mice and humans have two lineages of dendritic cells, where the dendritic cells have their origins in pre-dendritic cells (pre-DC1 and pre-DC2). Both humans and mice have pre-DC1 cells and pre-DC2 cells. The pre-DC1 cells mature into CD11c⁺CD8alpha⁺CD11b⁻ DCs, which have the property of inducing TH1-type immune response. The pre-DC2 cells mature into CD11c⁺CD8alpha⁻CD11b⁺ DCs, which have the property of inducing TH2-type immune response (Boonstra, et al. (2003) J. Exp. Med. 197:101-109; Donnenberg, et al. (2001) Transplantation 72:1946-1951; Becker (2003) Virus Genes 26:119-130). Mice and humans both have plasmacytoid dendritic cells (pDCs), where both mouse and human pDCs express interferon-alpha in response to viral stimulation (Carine, et al. (2003) J. Immunol. 171:6466-6477). Moreover, both the mouse and humans have myeloid DC where, for example, both mouse and human myeloid DCs can express CCL17 (Penna, et al. (2002) J. Immunol. 169:6673-6676; Alferink, et al. (2003) J. Exp. Med. 197:585-599).

Both mice and humans have CD8⁺ T cells. Both mouse and human CD8⁺ T cells comprise similar subsets, that is, central memory T cells and effector memory T cells (see, e.g., Walzer, et al. (2002) J. Immunol. 168:2704-2711). Immune response of CD8⁺ T cells are similar for both mouse and human CD8⁺ T cells as it applies, for example, to expression of CD127 and IL-2 (Fuller, et al. (2005) J. Immunol. 174:5926-5930).

The following narrative concerns Listeria-induced maturation of DCs. L. monocytogenes stimulates the maturation of both human and murine dendritic cells, as measured by Listerial-stimulated expression of, e.g., CD86 (see, e.g., Kolb-maurer, et al. (2000) Infection Immunity 68:3680-3688; Brzoza, et al. (2004) J. Immunol. 173:2641-2651; Esplugues, et al. (2005) Blood Feb. 3 (epub ahead of print); Paschen, et al. (2000) Eur. J. Immunol. 30:3447-3456).

Neutrophils of both the mouse and human are stimulated by Listeria (see, e.g., Kobayashi, et al. (2003) Proc. Natl. Acad. Sci. USA 100: 10948-10953; Torres, et al. (2004) 72:2131-2139; Sibelius, et al. (1999) Infection Immunity 67:1125-1130; Tvinnereim, et al. (2004) J. Immunol. 173:1994-2002). Neutrophils can be detected or characterized by the marker Gr-1 (also known as Gr1 and Ly-6G). Methods for measuring Gr-1 are available (see, e.g., Dumortier, et al. (2003) Blood 101:2219-2226; Bliss, et al. (2000) J. Immunol. 165:4515-4521).

Toll-like receptors (TLRs) comprise a family of about ten receptors, mediating innate response to bacterial components, viral components, and analogues thereof, including lipopolysaccharide (LPS), lipoteichoic acids, peptidoglycan components, lipoprotein, nucleic acids, flagellin, and CpG-DNA. Both humans and mice express the following toll-like receptors: TLR1, TLR2, TLR3, TLR4, TLR5, TLR6, TLR7, TLR8, and TLR9 (Janssens and Beyaert (2003) Clinical Microb. Revs. 16:637-646).

Response to L. monocytogenes, by mouse and human systems, involves expression of IFN-gamma (see, e.g., Way and Wilson (2004) J. Immunol. 173:5918-5922; Ouadrhiri, et al. (1999) J. Infectious Diseases 180:1195-1204; Neighbors, et al. (2001) J. Exp. Med. 194:343-354; Calorini, et al. (2002) Clin. Exp. Metastasis 19:259-264; Andersson, et al. (1998) J. Immunol. 161:5600-5606).

Response to L. monocytogenes, by both mouse and human systems, involves expression of tumor necrosis factor (TNF) (see, e.g., Flo, et al. (2000) J. Immunol. 164:2064-2069; Calorini, et al. (2002) Clin. Exp. Metastasis 19:259-264; Brzoza, et al. (2004) J. Immunol. 173:2641-2651).

Response to L. monocytogenes, as shown by murine and human studies, involves expression of interleukin-12 (IL-12) (see, e.g., Brzoza, et al. (2004) J. Immunol. 173:2641-2651; Cleveland, et al. (1996) Infection Immunity 64:1906-1912; Andersson, et al. (1998) J. Immunol. 161:5600-5606).

CD69 is an activation marker of immune cells, as determined in studies of mouse and human immune cells (see, e.g., Pisegna, et al. (2002) J. Immunol. 169:68-74; Gerosa, et al. (2002) J. Exp. Med. 195:327-333; Borrego, et al. (1999) Immunology 97:159-165).

The following concerns cytokines, e.g., interferon-gamma and MCP-1. Interferon-gamma (IFN-gamma) is expressed by both humans and mice. IFN-gamma is a key cytokine in the immune system's response against tumors and microbial pathogens, as well as against tumor angiogenesis. IFN-gamma mediates immune response against liver tumors and viral hepatitis, for example, by studies administering vaccines against hepatitis virus, administration of IFN-gamma, or administering anti-IFN antibodies (See, e.g., Grassegger and Hopfl (2004) Clin. Exp. Dermatol. 29:584-588; Tannenbaum and Hamilton (2000) Semin. Cancer Biol. 10:113-123; Blankensetein and Qin (2003) Curr. Opin. Immunol. 15:148-154; Fidler, et al. (1985) J. Immunol. 135:4289-4296; Okuse, et al. (2005) Antiviral Res. 65:23-34; Piazzolla, et al. (2005) J. Clin. Immunol. 25:142-152; Xu, et al. (2005) Vaccine 23:2658-2664; Irie, et al. (2004) Int. J. Cancer 111:238-245).

Monocyte chemoattractant protein (MCP-1; CCL2) is expressed by humans and mice. MCP-1 promotes macrophage infiltration of tumors. MCP-1 is mediates immune response to viral hepatitis infections. Moreover, administered MCP-1 promotes tumors eradication by macrophages. In other studies, MCP-1 was correlated with efficiency of drug therapy against viral hepatitis (See, e.g., Nakamura, et al. (2004) Cancer Gene Ther. 11:1-7; Luo, et al. (1994) J. Immunol. 153:3708-3716; Panasiuk, et al. (2004) World J. Gastroenterol. 10:36639-3642).

Immune response can involve response to proteins, peptides, cells expressing proteins or peptides, as well as against other entities such as nucleic acids, oligosaccharides, glycolipids, and lipids. For example, immune response against a virus can include immune response against a peptide of the virus, a nucleic acid of the virus, a glycolipid of the virus, or oligosaccharide of the virus (see, e.g., Rekvig, et al. (1995) Scand. J. Immunol. 41:593-602; Waisman, et al. (1996) Cell Immunol. 173:7-14; Cerutti, et al. (2005) Mol. Immunol. 42:327-333; Oschmann, et al. (1997) Infection 25:292-297; Paradiso and Lindberg (1996) Dev. Biol. Stand. 87:269-275).

A broad spectrum of tumors, viruses, bacteria, and other pathogens, are attacked by NK cells and NKT cells. The targets of NK cells and NKT cells include, e.g., colon adenocarcinomas, neuroblastomas, sarcomas, lymphomas, breast cancers, melanomas, erythroleukemic tumors, leukemias, mastocytomas, colon carcinomas, breast adenocarcinomas, ovarian adenocarcinomas, fibrosarcomas, melanomas, lung carcinomas, rhabdomyosarcomas, gliomas, renal cell cancers, gastric cancers, lung small cell carcinomas, cancers arising from metastasis to the liver, as well as a range of viruses, including, hepatitis A virus, hepatitis B virus, hepatitis C virus, herpes simplex virus, gamma herpes viruses, Epstein-Barr virus (EBV), HIV, dengue virus, and a range of bacteria, such as Mycoplasma, and Brucella (see, e.g., Vujanovic, et al. (1996) J. Immunol. 157:1117-1126; Kashii, et al. (1999) J. Immunol. 163:5358-5366; Giezeman-Smits, et al. (1999) J. Immunol. 163:71-76; Turner, et al. (2001) J. Immunol. 166:89-94; Kawarada, et al. (2001) J. Immunol. 167:5247-5253; Scott-Algara and Paul (2002) Curr. Mol. Med. 2:757-768; Karnbach, et al. (2001) J. Immunol. 167:2569-2576; Westwood, et al. (2003) J. Immunol. 171:757-761; Roda, et al. (2005) J. Immunol. 175:1619-1627; Poggi, et al. (2005) J. Immunol. 174:2653-2660; Metelitsa, et al. (2001) J. Immunol. 167:3114-3122; Wei, et al. (2000) J. Immunol. 165:3811-3819; Bakker, et al. (1998) J. Immunol. 160:5239-5245; Makrigiannis, et al. (2004) J. Immunol. 172:1414-1425; Golding, et al. (2001) Microbes Infect. 3:43-48; Lai, et al. (1990) J. Infect. Dis. 161:1269-1275; Ohga, et al. (2002) Crit. Rev. Oncol. Hematol. 44:203-215; Wakimoto, et al. (2003) Gene Ther. 10:983-990; Chen, et al. (2005) J. Viral Hepat. 12:38-45; Baba, et al. (1993) J. Clin. Lab Immunol. 40:47-60; Li, et al. (2004) J. Leukoc. Biol. 76:1171-1179; Scalzo (2002) Trends Microbiol. 10:470-474; Ahlenstiel and Rehermann (2005) Hepatology 41:675-677; Chen, et al. (2005) J. Viral Hepat. 12:38-45; Sun and Gao (2004) Gasteroenterol. 127:1525-1539; Li, et al. (2004) J. Leukoc. Biol. 76:1171-1179; Ahmad and Alvarez (2004) J. Leukoc. Biol. 76:743-759; Cook (1997) Eur. J. Gasteroenterol. Hepatol. 9:1239-1247; Williams and Riordan (2000) J. Gasteroenterol. Hepatol. 15 (Suppl.) G17-G25; Varani and Landini (2002) Clin. Lab. 48:39-44; Rubin (1997) Clin. Liver Dis. 1:439-452; Loh, et al. (2005) J. Virol. 79:661-667; Shresta, et al. (2004) Virology 319:262-273; Fjaer, et al. (2005) Pediatr. Transplant 9:68-73; Li, et al. (2004) World J. Gasteroenterol. 10:3409-3413; Collin, et al. (2004) J. Hepatol. 41:174-175; Ohga, et al. (2002) Crit. Rev. Oncol. Hematol. 44:203-215).

The invention encompasses methods of stimulating the NK cell-mediated killing of target cells, to provide a non-limiting example, where the target cells have reduced expression of an inhibiting ligand, and where the inhibiting ligand can be MHC Class I. NK cells lyse a broad range of target cells such as cancer cells and virus-infected cells, where NK cell-mediated lysis increases where the target cells have low expression of MHC Class I. Many or most tumor cells, cells infected with oncogenic viruses, and cells infected by non-oncogenic viruses, show low expression of MHC Class I. CT26 cells, MC38 cells, and YAC-1 cells, can express low levels of MHC Class I (see, e.g., Tardif and Siddiqui (2003) J. Virol. 77:11644-11650; Imboden, et al. (2001) Cancer Res. 61:1500-1507; Matsui, et al. (2001) Biochem. Biophys. Res. Commun. 285:508-517; Yoon, et al. (2001) Anticancer Res. 21:4031-4040; Bubenik (2003) Oncol. Rep. 10:2005-2008; Diefenbach and Raulet (2002) Immunol. Rev. 188:9-21; Khakoo, et al. (2004) Science 305:872-873; Parham (2004) Science 305:786-787). YAC-1 cells are a prototypic target of NK cells, widely used in experiments with NK cell-mediated lysis (see, e.g., Katsumoto, et al. (2004) J. Immunol. 173:4967-4975; Yan, et al. (2004) Immunology 112:105-116; Hashimoto, et al. (2003) Int. J. Cancer 103:508-513; Matsumoto, et al. (2000) Eur. J. Immunol. 30:3723-3731). CT26 cells and MC38 cells express low levels of MHC Class I (Seong, et al. (2001) Anticancer Res. 21:4031-4039; Su, et al. (2001) Biochim. Biophys. Res. Commun. 280:503-512). CT26 tumor cells are from Balb/c mice, whereas MC38 tumor cells are from C57Bl/6 mice. Balb/c mice are H-2d, and express 2 Kd, 2Ld, and 2Dd MHC types of MHC Class I molecules. C57BL/6 mice are H-2b, and express 2 Kb and 2 Db types of MHC Class I molecules (see, e.g., Skobeme, et al. (2002) J. Immunol. 169:1410-1418; Geginat, et al. (2001) J. Immunol. 166:1877-1884). Balb/c mice are Th2 type responders whereas C57Bl/6 mice are Th1 type responders. MC38 tumor cells have been described (see, e.g., Feldman, et al. (2000) Cancer Res. 60:1503-1506; Wildner, et al. (1999) Cancer Res. 59:5233-5238).

NK cells also can eliminate a broad range of parasitic organisms and protozoans, such as those responsible for toxoplasmosis, trypanosomiasis, leishmaniasis, and malaria (see, e.g., Korbel, et al. (20040 Int. J. Parasitol. 34:1517-1528; Mavoungou, et al. (2003) Eur. Cytokine Netw. 14:134-142; Doolan and Hoffman (1999) J. Immunol. 163:884-892).

In some embodiments of the invention, administration of the Listeria in the methods described herein stimulates an innate immune response. For instance, the invention provides methods of using Listeria to stimulate an NK-mediated innate immune response (e.g., an NK-mediated anti-tumor response). In some embodiments, administration of the Listeria to the mammal stimulates an acquired immune response. (The terms “adaptive immune response” and “acquired immune response” are used interchangeably herein.) In some embodiments, the adaptive immune response comprises a systemic, tumor-specific memory response. In some embodiments, the adaptive immune response is a CD4⁺ immune response and/or a CD8⁺ immune response. In some embodiments, administration of the Listeria stimulates both an innate immune response, as well as an acquired immune response. In some embodiments, the immune response (be it an innate and/or adaptive response) effects a reduction in one, or in any combination of, the following: number of tumors or cancer cells, tumor mass, and titer of an infectious agent. In some embodiments, the reduction is relative to the number, mass, or titer prior to administration of the Listeria to the mammal.

In some embodiments, the administering of the Listeria to the mammal stimulates one, or any combination, of a: a. NK cell; b. NKT cell; c. dendritic cell (DC); d. monocyte or macrophage; e. neutrophil; or f. toll-like receptor (TLR) or nucleotide-binding oligomerization domain (NOD) protein (e.g., as compared with immune response in the absence of the administering of the effective amount of the attenuated Listeria). In some embodiments, the immune response resulting from administration of the Listeria to the mammal activates NK cells. In some embodiments, administration of the Listeria to the mammal results in an increased number of NK cells in the liver and/or an increased percentage of NK cells among leukocytes in the liver (relative to the mammal prior to administration of the Listeria).

The present invention also encompasses methods in which the mammal comprises hepatic leukocytes, and the administering stimulates one or both of: a. an increase in the percent of hepatic leukocytes that are NK cells, compared to the percent without the administering of the attenuated Listeria; or b. an increase in expression of an activation marker by a hepatic NK cell, compared to the expression without (or prior to) the administering of the attenuated Listeria. Moreover, the invention further provides methods in which the increase in the percent of hepatic leukocytes that are NK cells is at least: a. 5%; b. 10%; c. 15%; d. 20%; or e. 25%, greater than compared to the percent without (or prior to) the administering of the attenuated Listeria.

Embraced by the present invention, are methods in which the administering of the Listeria to the mammal stimulates increased expression of any one, or any combination, of: a. CD69; b. interferon-gamma (IFNgamma); c. interferon-alpha (IFNalpha) or interferon-beta (IFNbeta); d. interleukin-12 (IL-12), monocyte chemoattractant protein (MCP-1), or e. interleukin-6 (IL-6) (e.g., compared with expression in the absence of the administering of the effective amount of the attenuated Listeria).

III. Treating Infections.

The present invention, in some embodiments, supplies methods and reagents for stimulating immune response to infections, e.g., infections of the liver. Infectious conditions encompass viral infections, bacterial infections, fungal infections, and parasitic infestations. In some embodiments, the infectious conditions are non-Listerial infectious conditions. In some embodiments, the infections are in the liver. Possible infectious agents likewise include viruses, bacteria, fungi, and parasites. In some embodiments, the infectious agents are non-Listerial infectious agents. In some embodiments, the infectious agents are hepatotropic.

In some embodiments, the infectious conditions include infections from hepatotropic viruses and viruses that mediate hepatitis, e.g., hepatitis B virus, hepatitis C virus, and cytomegalovirus. The invention contemplates methods to treat other hepatotropic viruses, such as herpes simplex virus, Epstein-Barr virus, and dengue virus. NK cells, for example, have been shown to mediate immune response against these viruses (see, e.g., above citations).

In some embodiments, the infectious agent is selected from the group consisting of Human Immunodeficiency virus; Feline Immunodeficiency virus; herpes simplex virus (HSV) type 1 and 2; cytomegalovirus; metapneumovirus; Epstein-Barr virus; Varicella Zoster Virus; hepatitis B virus; hepatitis A virus; hepatitis C virus; delta hepatitis virus; hepatitis E virus; and hepatitis G virus. In further embodiments, the infectious agent is a virus from any one of the families Picornaviridae (e.g., polioviruses, rhinoviruses, etc.); Caliciviridae; Togaviridae (e.g., rubella virus, dengue virus, etc.); Flaviviridae; Coronaviridae; Reoviridae (e.g., rotavirus, etc.); Birnaviridae; Rhabodoviridae (e.g., rabies virus, etc.); Orthomyxoviridae (e.g., influenza virus types A, B and C, etc.); Filoviridae; Paramyxoviridae (e.g., mumps virus, measles virus, respiratory syncytial virus, parainfluenza virus, etc.); Bunyaviridae; Arenaviridae; Retroviradae; Papillomavirus, the tick-borne encephalitis viruses; and the like. See, e.g. Virology, 3rd Edition (W. K. Joklik ed. 1988); Fundamental Virology, 3rd Edition (B. N. Fields, D. M. Knipe, and P. M. Howley, Eds. 1996), for a description of these and other viruses.

In another aspect, the present invention provides methods and reagents for the treatment and/or prevention of parasitic infections, e.g., parasitic infections of the liver. These include, without limitation, liver flukes (e.g., Clonorchis, Fasciola hepatica, Opisthorchis), Leishmania, Ascaris lumbricoides, Schistosoma, and helminths. Helminths include, e.g., nematodes (roundworms), cestodes (tapeworms), and trematodes (flatworms or flukes). NK cells, as well as other immune cells, respond to these infections (see, e.g., Tliba, et al. (2002) Vet. Res. 33:327-332; Keiser and Utzinger (2004) Expert Opin. Pharmacother. 5:1711-1726; Kaewkes (2003) Acta Trop. 88:177-186; Srivatanakul, et al. (2004) Asian Pac. J. Cancer Prev. 5:118-125; Stuaffer, et al. (2004) J. Travel Med. 11:157-159; Nylen, et al. (2003) Clin. Exp. Immunol. 131:457-467; Bukte, et al. (2004) Abdom. Imaging 29:82-84; Singh and Sivakumar (2003) 49:55-60; Wyler (1992) Parisitol. Today 8:277-279; Wynn, et al. (2004) Immunol. Rev. 201:156-167; Asseman, et al. (1996) Immunol. Lett. 54:11-20; Becker, et al. (2003) Mol. Biochem. Parasitol. 130:65-74; Pockros and Capozza (2005) Curr. Infect. Dis. Rep. 7:61-70; Hsieh, et al. (2004) J. Immunol. 173:2699-2704; Korten, et al. (2002) J. Immunol. 168:5199-5206; Pockros and Capozza (2004) Curr. Gastroenterol. Rep. 6:287-296).

Yet another aspect of the present invention provides methods and reagents for the treatment and/or prevention of bacterial infections, e.g., by hepatotropic bacteria. Provided are methods and reagents for treating, e.g., Mycobacterium tuberculosis, Treponema pallidum, and Salmonella spp. NK cells, as well as other cells of the immune system, respond to these bacterial infections (see, e.g., Cook (1997) Eur. J. Gasteroenterol. Hepatol. 9:1239-1247; Vankayalapati, et al. (2004) J. Immunol. 172:130-137; Sellati, et al. (2001) J. Immunol. 166:4131-4140; Jason, et al. (2000) J. Infectious Dis. 182:474-481; Kirby, et al. (2002) J. Immunol. 169:4450-4459; Johansson and Wick (2004) J. Immunol. 172:2496-2503; Hayashi, et al. (2004) Intern. Med. 43:521-523; Akcay, et al. (2004) Int. J. Clin. Pract. 58:625-627; de la Barrera, et al. (2004) Clin. Exp. Immunol. 135:105-113). In some embodiments, the infectious agent is a bacterial pathogen such as Mycobacterium, Bacillus, Yersinia, Salmonella, Neisseria, Borrelia, Chlamydia, or Bordetella. In one embodiment, the infectious agent is Mycobacterium tuberculosis, Bacillus anthracis, or Yersinia pestis.

In some embodiments, the infectious condition comprises one or more of: a. hepatitis B; b. hepatitis C; c. human immunodeficiency virus (HIV); d. cytomegalovirus (CMV); e. Epstein-Barr virus (EBV); or f. leishmaniasis. Likewise, in some embodiments, the infectious agent is hepatitis B virus, hepatitis C virus, human immunodeficiency virus (HIV), cytomegalovirus (CMV), Epstein-Barr virus (EBV), or leishmaniasis. In some further embodiments, the infectious agent is a polyomavirus or human papillomavirus.

In some further embodiments, the infectious condition is selected from the group consisting of Diptheria, Pertussis, Tetanus, Tuberculosis, Bacterial or Fungal Pneumonia, Otitis Media, Gonorrhea, Cholera, Typhoid, Meningitis, Mononucleosis, Plague, Shigellosis or Salmonellosis, Legionaire's Disease, Lyme Disease, Leprosy, Malaria, Hookworm, Onchocerciasis, Schistosomiasis, Trypanosomiasis, Leishmaniasis, Giardia, Amoebiasis, Filariasis, Borelia, and Trichinosis.

IV. Listerial Genes and Proteins, Including Virulence Factors.

L. monocytogenes expresses various genes and gene products that contribute to growth or colonization in the host. Some of these genes and gene products are classed as “virulence factors.” The virulence factors facilitate bacterial infection of host cells. These virulence factors include actA, listeriolysin (LLO), protein 60 (p60), internalin A (inlA), internalin B (inlB), phosphatidylcholine phospholipase C (PC-PLC), phosphatidylinositol-specific phospholipase C (PI-PLC; plcA gene). A number of other internalins have been characterized, e.g., InlC2, INlD, InlE, and InlF (Dramsi, et al. (1997) Infect. Immunity 65:1615-1625). Mpl, a metalloprotease that processes propL-PLC to active PL-PLC, is also a virulence factor (Chakraborty, et al. (2000) Int. J. Med. Microbiol. 290:167-174; Williams, et al. (2000) J. Bact. 182:837-841). Some non-limiting examples of nucleic acid sequences encoding these virulence factors, as well as a number of other factors that contribute to growth or to spread, are disclosed below. Without limiting the present invention to the list of embodiments disclosed in Table 1, the present invention supplies a Listeria that is altered, mutated, or attenuated in one or more of the sequences of Table 1. Table 1 enables one of ordinary skill in the art to identify corresponding genes or coding sequences in various strains of L monocytogenes, and to prepare an attenuated L. monocytogenes for use in treating a cancer, tumor, precancerous disorder, or infection, e.g., of the liver.

TABLE 1
L. monocytogenes nucleic acids and proteins.

| Protein/Gene | Nucleotides | GenBank Acc. No. |
|---|---|---|
| Actin assembly inducing | 209470-211389 (coding | NC_003210 |
| protein precursor (ActA | sequence) | |
| gene) | 209456-211389 (gene) | |
| actA in various | — | AF497169; AF497170; |
| L. monocytogenes subtypes. | | AF497171; AF497172; |
| | | AF497173; AF497174; |
| | | AF497175; AF497176; |
| | | AF497177; AF497178; |
| | | AF497179; AF497180; |
| | | AF497181; AF497182; |
| | | AF497183 (Lasa, et al. |
| | | (1995) Mol. Microbiol. |
| | | 18: 425-436). |
| Listeriolysin O precursor | 205819-207408 | NC_003210 |
| (LLO) (hly gene) | | |
| Internalin A (InlA) | 454534-456936 | NC_003210 |
| Internalin B (InlB) | 457021-458913 | NC_003210 |
| SvpA | — | Bierne, et al. (2004) J. |
| | | Bacteriol. 186: 1972-1982; |
| | | Borezee, et al. (2000) |
| | | Microbiology 147: 2913-2923. |
| p104 (a.k.a. LAP) | | Pandiripally, et al. (1999) J. |
| | | Med. Microbiol. 48: 117-124; |
| | | Jaradat, et al. (2003) |
| | | Med. Microbiol. Immunol. |
| | | 192: 85-91. |
| Phosphatidylinositol- | 204624-205577 | NC_003210 |
| specific phospholipase C | | |
| (PI-PLC) (plcA gene) | | |
| Phosphatidylcholine- | 1-3031 | X59723 |
| specific phospholipase C | | |
| (PC-PLC) (plcB gene) | | |
| Zinc metalloprotease | 207739-209271 | NC_003210 |
| precursor (Mpl) | | |
| p60 (protein 60; invasion | Complement of | NC_003210 (Lenz, et al. |
| associated protein (iap)). | 618932-620380 | (2003) Proc. Natl. |
| | | Acad. Sci. |
| | | USA 100: 12432-12437). |
| Sortase | 966245-966913 | NC_003210 |
| Listeriolysin positive | 203607-203642 | NC_003210 |
| regulatory protein (PrfA | | |
| gene) | | |
| Listeriolysin positive | 1-801 | AY318750 |
| regulatory protein (PrfA | | |
| gene) | | |
| PrfB gene | 2586114-2587097 | NC_003210 |
| FbpA gene | 570 amino acids | Dramsi, et al. (2004) Mol. |
| | | Microbiol. 53: 639-649. |
| Auto gene | — | Cabanes, et al. (2004) Mol. |
| | | Microbiol. 51: 1601-1614. |
| Ami (amidase that mediates | — | Dussurget, et al. (2004) |
| adhesion) | | Annu. Rev. Microbiol. |
| | | 58: 587-610. |
| dlt operon (dltA; dltB; dltC; | 487-2034 (dltA) | GenBank Acc. No: |
| dltD). | | AJ012255 (Abachin, et al. |
| | | (2002) Mol. Microbiol. |
| | | 43: 1-14.) |
| prfA boxes | — | Table 1 of Dussurget, et al. |
| | | (2002) Mol. Microbiol. |
| | | 45: 1095-1106. |
| Htp (sugar-P transporter) | 1-1386 | GenBank Acc. No. |
| | | AJ315765 (see, e.g., |
| | | Milohanic, et al. (2003) |
| | | Mol. Microbiol. 47: 1613-1625). |

The referenced nucleic acid sequences, and corresponding translated amino acid sequences, and the cited amino acid sequences, and the corresponding nucleic acid sequences associated with or cited in that reference, are incorporated by reference herein in their entirety.

Listeriolysin (LLO), encoded by the hly gene, mediates escape of the bacterium from the phagolysosome and into the cytoplasm of the host cell. LLO also mediates effective transfer of the bacterium from one host cell to a neighboring host cell. During spread, LLO mediates escape of the bacterium from a double membrane vesicle into the cytoplasm of the neighboring cell (see, e.g., Glomski, et al. (2003) Infect. Immun. 71:6754-6765; Gedde, et al. (2000) Infect. Immun. 68:999-1003; Glomski, et al. (2002) J. Cell Biol. 156:1029-1038; Dubail, et al. (2001) Microbiol. 147:2679-2688; Dramsi and Cosssart (2002) J. Cell Biol. 156:943-946).

ActA is a protein of Listeria's surface that recruits the host cell's actin. In other words, Act A serves as a scaffold to assemble host cell actin and other proteins of the cytoskeleton, where assembly occurs at the surface of the bacterium. ActA mediates propulsion of the Listeria through the host cell's cytoplasm. ActA mutants are able to escape from the phagocytic vacuole, but grow inside the host cytosol as “microcolonies” and do not spread from cell to cell (see, e.g., Machner, et al. (2001) J. Biol. Chem. 276:40096-40103; Lauer, et al. (2001) Mol. Microbiol. 42:1163-1177; Portnoy, et al. (2002) J. Cell Biol. 158:409-414).

Internalin A is a ligand for the mammalian membrane-bound protein, E-cadherin. Internalin B is a ligand for a small number of mammalian membrane-bound proteins, e.g., Met receptor (also known as HGF-R/Met) and gClq-R, and proteoglycans. L. monocytogenes can express about 24 members of the internalin-related protein family, including, e.g., an internalin encoded by the irpA gene (see, e.g., Bierne and Cossart (2000) J. Cell Sci. 115:3357-3367; Schluter, et al. (1998) Infect. Immun. 66:5930-5938; Dormann, et al. (1997) Infect. Immun. 65:101-109).

Sortase proteins catalyze the processing and maturation of internalin A. Two sortases have been identified in L. monocytogenes, srtA and srtB. The srtA mutant is defective in bacterial internalization, as determined in studies with human enterocytes and hepatocytes. Hence, mature internalin A is needed for uptake by enterocytes and hepatocytes. The srtA mutant can still be taken up by cells that are able to utilize other mechanisms of uptake, such as the internalin, e.g., InlB (see, e.g., Bierne, et al. (2002) Mol. Microbiol. 43:869-881).

Two phospholipases, PI-PLC (encoded by plcA gene) and PC-PLC (encoded by plcB gene), are also among the virulence factors. PI-PLC mediates lysis of the host phagosome, allowing escape of the bacterium into the cytosol. Bacterial mutants in PC-PLC show reduced virulence and are found to accumulate within the double-membrane vesicles that mediate cell-to-cell transmission (see, e.g., Camilli, et al. (1993) Mol. Microbiol. 8:143-157; Schulter, et al. (1998) Infect. Immun. 66:5930-5938).

Protein p60, encoded by the iap gene, mediates intracellular movement and cell-to-cell spread. Intracellular movement and spread in iap gene mutants are much reduced (Pilgrim, et al. (2003) Infect. Immun. 71:3473-3484).

The invention also contemplates a Listeria attenuated in at least one regulatory factor, e.g., a promoter or a transcription factor. ActA expression is regulated by two different promoters, one immediately upstream of actA and the second in front of the mpl gene, upstream of actA (Lauer, et al. (2002) J. Bacteriol. 184:4177-4186). The present invention, in certain embodiments, provides a nucleic acid encoding inactivated, mutated, or deleted in at least one actA promoter. The transcription factor prfA is required for transcription of a number of L. monocytogenes genes, e.g., hly, plcA, actA, mpl, prfA, and iap. PrfA's regulatory properties are mediated by, e.g., the PrfA-dependent promoter (PinlC) and the PrfA-box. The present invention, in some embodiments, provides a nucleic acid encoding inactivated, mutated, or deleted in at least one of PrfA, PinlC, PrfA-box, and the like (see, e.g., Lalic-Mullthaler, et al. (2001) Mol. Microbiol. 42:111-120; Shetron-Rama, et al. (2003) Mol. Microbiol. 48:1537-1551; Luo, et al. (2004) Mol. Microbiol. 52:39-52). Together, inlA and inlB are regulated by five promoters (Lingnau, et al. (1995) Infect. Immun. 63:3896-3903). The invention, in certain embodiments, provides a Listeria attenuated in one or more of these promoters.

The invention also supplies a Listeria bacterium that is attenuated by treatment with a DNA cross-linking agent (e.g., psoralen) and by inactivating at least one gene that mediates DNA repair, e.g., a recombinational repair gene (e.g., recA) or an ultraviolet light damage repair gene (e.g., uvrA, uvrB, uvrAB, uvrC, uvrD, phrA, phrB) (see, e.g., U.S. Pat. Publication No. 2004/0228877 of Dubensky, et al. and U.S. Pat. Publication No. 2004/0197343 of Dubensky, et al.).

The Listeria of the present invention be engineered, e.g., by way of a plasmid-based construct and/or genomic construct, to comprise an antibiotic resistance gene or antibiotic resistance marker, e.g., as part of the listerial genome or as a plasmid. The antibiotic resistance gene can be, e.g., chloramphenicol acetyltransferase; penicillin-binding protein 2; erythromycin resistance determinant; penicillin beta-lactamase; or aminoglycoside acetyltransferase (see, e.g., Guo, et al. (1997) Nature 389:40-46; Langer, et al. (2002) Nucleic Acids Res. 30:3067-3077; Grindley (1997) Curr. Biol. 7:R608-R612; Qian, et al. (1992) J. Biol. Chem. 267:7794-7805; New England Biolabs (2005) Catalogue, New Engl. Biolabs, Beverly, Mass., p. 20).

V. Listeria.

In some embodiments, the Listeria belong to the species Listeria monocytogenes. In some alternative embodiments the bacteria are members of the Listeria ivanovii, Listeria seeligeri, Listeria innocua, L. Welshimeri, or L. grayi species.

In some embodiments, the Listeria are non-naturally occurring. In some embodiments, the Listeria are mutant Listeria, recombinant Listeria, or otherwise modified. In some embodiments, the Listeria are attenuated. In some embodiments, the Listeria are metabolically active. In some embodiments, the Listeria are capable of cytosolic entry (i.e., capable of accessing the cytosol from a phagocytic vacuole in a cell).

In some embodiments, the attenuated Listeria is attenuated in one or more of growth, cell to cell spread, binding to or entry into a host cell, replication, or DNA repair. In some embodiments, the Listeria is attenuated by one or more of an actA mutation, an inlB mutation, a uvrA mutation, a uvrB mutation, a uvrC mutation, a nucleic acid targeting compound, or a uvrAB mutation and a nucleic acid targeting compound. In some embodiments, the attenuated Listeria is attenuated in cell to cell spread and/or entry into nonphagocytic cells. In some embodiments, the Listeria is attenuated by one or more of an actA mutation or an actA mutation and an inlB mutation. In some embodiments, the Listeria is ΔactA or ΔactAΔinlB.

In some embodiments, the attenuated Listeria is attenuated for cell-to-cell spread. In some embodiments, the Listeria attenuated for cell-to-cell spread are defective with respect to ActA (e.g., relative to the non-modified or wild-type Listeria). In some embodiments, the Listeria comprises an attenuating mutation in the actA gene. In some embodiments, the Listeria comprises a full or partial deletion in the actA gene.

In some embodiments, the capacity of the attenuated Listeria bacterium for cell-to-cell spread is reduced by at least about 10%, at least about 25%, at least about 50%, at least about 75%, or at least about 90%, relative to Listeria without the attenuating mutation (e.g., wild type Listeria). In some embodiments, the capacity of the attenuated Listeria bacterium for cell-to-cell spread is reduced by at least about 25% relative to Listeria without the attenuating mutation. In some embodiments, the capacity of the attenuated Listeria bacterium attenuated for cell-to-cell spread is reduced by at least about 50% relative to the Listeria without the attenuating mutation.

In vitro assays for determining whether a Listeria bacterium is attenuated for cell-to-cell spread are known to those of ordinary skill in the art. For example, the diameter of plaques formed over a time course after infection of selected cultured cell monolayers can be measured. Plaque assays within L2 cell monolayers can be performed as described previously in Sun, A., A. Camilli, and D. A. Portnoy. 1990, Isolation of Listeria monocytogenes small-plaque mutants defective for intracellular growth and cell-to-cell spread. Infect. Immun. 58:3770-3778, with modifications to the methods of measurement, as described by in Skoble, J., D. A. Portnoy, and M. D. Welch. 2000, Three regions within ActA promote Arp2/3 complex-mediated actin nucleation and Listeria monocytogenes motility. J. Cell Biol. 150:527-538. In brief, L2 cells are grown to confluency in six-well tissue culture dishes and then infected with bacteria for 1 h. Following infection, the cells are overlayed with media warmed to 40° C. that is comprised of DME containing 0.8% agarose, Fetal Bovine Serum (e.g., 2%), and a desired concentration of Gentamicin. The concentration of Gentamicin in the media dramatically affects plaque size, and is a measure of the ability of a selected Listeria strain to effect cell-to-cell spread (Glomski, I J., M. M. Gedde, A. W. Tsang, J. A. Swanson, and D. A. Portnoy. 2002. J. Cell Biol. 156:1029-1038). For example, in some embodiments at 3 days following infection of the monolayer the plaque size of Listeria strains having a phenotype of defective cell-to-cell spread is reduced by at least 50% as compared to wild-type Listeria, when overlayed with media containing Gentamicin at a concentration of 50 μg/ml. On the other hand, the plaque size between Listeria strains having a phenotype of defective cell-to-cell spread and wild-type Listeria is similar when infected monolayers are overlayed with media+agarose containing only 5 μg/ml gentamicin. Thus, the relative ability of a selected strain to effect cell-to-cell spread in an infected cell monolayer relative to wild-type Listeria can be determined by varying the concentration of gentamicin in the media containing agarose. Optionally, visualization and measurement of plaque diameter can be facilitated by the addition of media containing Neutral Red (GIBCO BRL; 1:250 dilution in DME+agarose media) to the overlay at 48 h. post infection. Additionally, the plaque assay can be performed in monolayers derived from other primary cells or continuous cells. For example HepG2 cells, a hepatocyte-derived cell line, or primary human hepatocytes can be used to evaluate the ability of selected Listeria mutants to effect cell-to-cell spread, as compared to wild-type Listeria. In some embodiments, Listeria comprising mutations or other modifications that attenuate the Listeria for cell-to-cell spread produce “pinpoint” plaques at high concentrations of gentamicin (about 50 μg/ml).

In some embodiments, the Listeria is attenuated for entry into non-phagocytic cells (relative or the non-mutant or wildtype Listeria). In some embodiments, the Listeria is defective with respect to one or more internalins (or equivalents). In some embodiments; the Listeria is defective with respect to internalin A. In some embodiments, the Listeria is defective with respect to internalin B. In some embodiments, the Listeria comprise a mutation in inlA. In some embodiments, the Listeria comprise a mutation in inlB. In some embodiments, the Listeria comprise a mutation in both actA and inlB. In some embodiments, the Listeria is deleted in functional ActA and internalinB. In some embodiments, the attenuated Listeria bacterium is an ΔactAΔinlB double deletion mutant. In some embodiments, the Listeria bacterium is defective with respect to both ActA and internalin B.

In some embodiments, the capacity of the attenuated Listeria bacterium for entry into non-phagocytic cells is reduced by at least about 10%, at least about 25%, at least about 50%, at least about 75%, or at least about 90%, relative to Listeria without the attenuating mutation (e.g., the wild type bacterium). In some embodiments, the capacity of the attenuated Listeria bacterium for entry into non-phagocytic cells is reduced by at least about 25% relative to Listeria without the attenuating mutation. In some embodiments, the capacity of the attenuated bacterium for entry into non-phagocytic cells is reduced by at least about 50% relative to Listeria without the attenuating mutation. In some embodiments, the capacity of the attenuated Listeria bacterium for entry into non-phagocytic cells is reduced by at least about 75% relative to Listeria without the attenuating mutation.

In some embodiments, the attenuated Listeria is not attenuated for entry into more than one type of non-phagocytic cell. For instance, the attenuated strain may be attenuated for entry into hepatocytes, but not attenuated for entry into epithelial cells. As another example, the attenuated strain may be attenuated for entry into epithelial cells, but not hepatocytes. It is also understood that attenuation for entry into a non-phagocytic cell of a particular modified Listeria is a result of mutating a designated gene, for example a deletion mutation, encoding an invasin protein which interacts with a particular cellular receptor, and as a result facilitates infection of a non-phagocytic cell. For example, Listeria ΔinlB mutant strains are attenuated for entry into non-phagocytic cells expressing the hepatocyte growth factor receptor (c-met), including hepatocyte cell lines (e.g., HepG2), and primary human hepatocytes.

In some embodiments, even though the Listeria is attenuated for entry into non-phagocytic cells, the Listeria is still capable of uptake by phagocytic cells, such as at least dendritic cells and/or macrophages. In one embodiment the ability of the attenuated Listeria to enter phagocytic cells is not diminished by the modification made to the strain, such as the mutation of an invasin (i.e. approximately 95% or more of the measured ability of the strain to be taken up by phagocytic cells is maintained post-modification). In other embodiments, the ability of the attenuated Listeria to enter phagocytic cells is diminished by no more than about 10%, no more than about 25%, no more than about 50%, or no more than about 75%.

In some embodiments of the invention, the amount of attenuation in the ability of the Listeria to enter non-phagocytic cells ranges from a two-fold reduction to much greater levels of attenuation. In some embodiments, the attenuation in the ability of the Listeria to enter non-phagocytic cells is at least about 0.3 log, about 1 log, about 2 log, about 3 log, about 4 log, about 5 log, or at least about 6 log. In some embodiments, the attenuation is in the range of about 0.3 to >8 log, about 2 to >8 log, about 4 to >8 log, about 6 to >8 log, about 0.3-8 log, also about 0.3-7 log, also about 0.3-6 log, also about 0.3-5 log, also about 0.3-4 log, also about 0.3-3 log, also about 0.3-2 log, also about 0.3-1 log. In some embodiments, the attenuation is in the range of about 1 to >8 log, 1-7 log, 1-6 log, also about 2-6 log, also about 2-5 log, also about 3-5 log.

In vitro assays for determining whether or not a Listeria bacterium is attenuated for entry into non-phagocytic cells are known to those of ordinary skill in the art. For instance, both Dramsi et al., Molecular Microbiology 16:251-261 (1995) and Gaillard et al., Cell 65:1127-1141 (1991) describe assays for screening the ability of mutant L. monocytogenes strains to enter certain cell lines. For instance, to determine whether a Listeria bacterium with a particular modification is attenuated for entry into a particular type of non-phagocytic cells, the ability of the attenuated Listeria bacterium to enter a particular type of non-phagocytic cell is determined and compared to the ability of the identical Listeria bacterium without the modification to enter non-phagocytic cells. Likewise, to determine whether a Listeria strain with a particular mutation is attenuated for entry into a particular type of non-phagocytic cells, the ability of the mutant Listeria strain to enter a particular type of non-phagocytic cell is determined and compared to the ability of the Listeria strain without the mutation to enter non-phagocytic cells. For instance, the ability of a modified Listeria bacterium to infect non-phagocytic cells, such as hepatocytes, can be compared to the ability of non-modified Listeria or wild type Listeria to infect phagocytic cells. In such an assay, the modified and non-modified Listeria is typically added to the non-phagocytic cells in vitro for a limited period of time (for instance, an hour), the cells are then washed with a gentamicin-containing solution to kill any extracellular bacteria, the cells are lysed and then plated to assess titer. Examples of such an assay are found in U.S. Patent Publication No. 2004/0228877. In addition, confirmation that the strain is defective with respect to internalin B may also be obtained through comparison of the phenotype of the strain with the previously reported phenotypes for internalin B mutants.

A Listeria monocytogenes ΔactAΔinlB strain was deposited with the American Type Culture Collection (ATCC), 10801 University Blvd., Manassas, Va. 20110-2209, United States of America (P.O. Box 1549, Manassas, Va., 20108, United States of America), on Oct. 3, 2003, under the provisions of the Budapest Treaty on the International Recognition of the Deposit of Microorganisms for the Purposes of Patent Procedure, and designated with accession number PTA-5562. Another Listeria monocytogenes strain, an ΔactAΔuvrAB strain, was also deposited with the ATCC on Oct. 3, 2003, under the provisions of the Budapest Treaty on the International Recognition of the Deposit of Microorganisms for the Purposes of Patent Procedure, and designated with accession number PTA-5563.

In some embodiments, Listeria is attenuated for nucleic acid repair (e.g., relative to wildtype). For instance, in some embodiments, the Listeria is defective with respect to at least one DNA repair enzyme (e.g., Listeria monocytogenes uvrAB mutants). In some embodiments, the Listeria is defective with respect to PhrB, UvrA, UvrB, UvrC, UvrD, and/or RecA. In some embodiments, the bacteria are defective with respect to UvrA, UvrB, and/or UvrC. In some embodiments, the bacteria comprise attenuating mutations in phrB, uvrA, uvrB, uvrC, uvrD, and/or recA genes. In some embodiments, the bacteria comprise one or more mutations in the uvrA, uvrB, and/or uvrC genes. In some embodiments, the bacteria are functionally deleted in UvrA, UvrB, and/or UvrC. In some embodiments, the bacteria are deleted in functional UvrA and UvrB. In some embodiments, the bacteria are uvrAB deletion mutants. In some embodiments, the bacteria are ΔuvrABΔactA mutants. In some embodiments, the nucleic acid of the bacteria which are attenuated for nucleic acid repair and/or are defective with respect to at least one DNA repair enzyme are modified by reaction with a nucleic acid targeting compound. Nucleic acid repair mutants, such as ΔuvrAB Listeria monocytogenes mutants, and methods of making the mutants, are described in detail in U.S. Patent Publication No. 2004/0197343, which is incorporated by reference herein in its entirety (see, e.g., Example 7 of U.S. 2004/0197343).

In some embodiments, the capacity of the attenuated Listeria bacterium for nucleic acid repair is reduced by at least about 10%, at least about 25%, at least about 50%, at least about 75%, or at least about 90%, relative to a Listeria bacterium without the attenuating mutation (e.g., the wild type bacterium). In some embodiments, the capacity of the attenuated Listeria bacterium for nucleic acid repair is reduced by at least about 25% relative to a Listeria bacterium without the attenuating mutation. In some embodiments, the capacity of the attenuated Listeria bacterium attenuated for nucleic acid repair is reduced by at least about 50% relative a Listeria bacterium without the attenuating mutation.

Confirmation that a particular mutation is present in a bacterial strain can be obtained through a variety of methods known to those of ordinary skill in the art. For instance, the relevant portion of the strain's genome can be cloned and sequenced. Alternatively, specific mutations can be identified via PCR using paired primers that code for regions adjacent to a deletion or other mutation. Southern blots can also be used to detect changes in the bacterial genome. Also, one can analyze whether a particular protein is expressed by the strain using techniques standard to the art such as Western blotting. Confirmation that the strain contains a mutation in the desired gene may also be obtained through comparison of the phenotype of the strain with a previously reported phenotype. For example, the presence of a nucleotide excision repair mutation such as deletion of uvrAB can be assessed using an assay which tests the ability of the bacteria to repair its nucleic acid using the nucleotide excision repair (NER) machinery and comparing that ability against wild-type bacteria. Such functional assays are known in the art. For instance, cyclobutane dimer excision or the excision of UV-induced (6-4) products can be measured to determine a deficiency in an NER enzyme in the mutant (see, e.g., Franklin et al., Proc. Natl. Acad. Sci. USA, 81: 3821-3824 (1984)). Alternatively, survival measurements can be made to assess a deficiency in nucleic acid repair. For instance, the Listeria can be subjected to psoralen/UVA treatment and then assessed for their ability to proliferate and/or survive in comparison to wild-type.

In some embodiments, the Listeria is capable of entering the cytosol from a phagocytic vacuole. In some embodiments, the ability of the Listeria to enter the cytosol is at least 5%, at least 10%, at least 25%, at least 50%, at least 75%, or at least 90% of a wild-type Listeria. Methods of assessing the degree to which a strain of Listeria is capable of cytosolic entry are known in the art. The methods include, but are not limited to, electron microscopy (Gedde et al., Infection and Immunity, 68:999-1003 (2000) and Tilney et al., J. Cell Biology, 109:1597-1608 (1989), each incorporated by reference herein) and phagosomal escape assays utilizing indirect immunofluorescence (Glomski et al., Infection and Immunity, 71:6754-6765 (2003) and Glomski et al., J. Cell Biology, 156:1029-1038 (2002), each of which is incorporated by reference herein).

The invention supplies a number of Listeria strains for making or engineering an attenuated Listeria of the present invention (Table 2). The Listeria of the present invention are not to be limited by the strains disclosed in this table.

TABLE 2
Strains of Listeria for use in the present invention.

| L. monocytogenes 10403S wild type. | Bishop and Hinrichs (1987) J. Immunol. |
|---|---|
| | 139: 2005-2009; Lauer, et al. (2002) J. Bact. |
| | 184: 4177-4186. |
| L. monocytogenes DP-L4056 (phage cured). The | Lauer, et al. (2002) J. Bact. 184: 4177-4186. |
| prophage-cured 10403S strain is designated DP- | |
| L4056. | |
| L. monocytogenes DP-L4027, which is DP-L2161, | Lauer, et al. (2002) J. Bact. 184: 4177-4186; Jones |
| phage cured, deleted in hly gene. | and Portnoy (1994) Infect. Immunity 65: 5608-5613. |
| L. monocytogenes DP-L4029, which is DP-L3078, | Lauer, et al. (2002) J. Bact. 184: 4177-4186; |
| phage cured, deleted in actA. | Skoble, et al. (2000) J. Cell Biol. 150: 527-538. |
| L. monocytogenes DP-L4042 (delta PEST) | Brockstedt, et al. (2004) Proc. Natl. Acad. Sci. |
| | USA 101: 13832-13837; supporting information. |
| L. monocytogenes DP-L4097 (LLO-S44A). | Brockstedt, et al. (2004) Proc. Natl. Acad. Sci. |
| | USA 101: 13832-13837; supporting information. |
| L. monocytogenes DP-L4364 (delta lplA; lipoate | Brockstedt, et al. (2004) Proc. Natl. Acad. Sci. |
| protein ligase). | USA 101: 13832-13837; supporting information. |
| L. monocytogenes DP-L4405 (delta inlA). | Brockstedt, et al. (2004) Proc. Natl. Acad. Sci. |
| | USA 101: 13832-13837; supporting information. |
| L. monocytogenes DP-L4406 (delta inlB). | Brockstedt, et al. (2004) Proc. Natl. Acad. Sci. |
| | USA 101: 13832-13837; supporting information. |
| L. monocytogenes CS-L0001 (delta actA-delta | Brockstedt, et al. (2004) Proc. Natl. Acad. Sci. |
| inlB). | USA 101: 13832-13837; supporting information. |
| L. monocytogenes CS-L0002 (delta actA-delta | Brockstedt, et al. (2004) Proc. Natl. Acad. Sci. |
| lplA). | USA 101: 13832-13837; supporting information. |
| L. monocytogenes CS-L0003 (L461T-delta lplA). | Brockstedt, et al. (2004) Proc. Natl. Acad. Sci. |
| | USA 101: 13832-13837; supporting information. |
| L. monocytogenes DP-L4038 (delta actA-LLO | Brockstedt, et al. (2004) Proc. Natl. Acad. Sci. |
| L461T). | USA 101: 13832-13837; supporting information. |
| L. monocytogenes DP-L4384 (S44A-LLO | Brockstedt, et al. (2004) Proc. Natl. Acad. Sci. |
| L461T). | USA 101: 13832-13837; supporting information. |
| L. monocytogenes. Mutation in lipoate protein | O'Riordan, et al. (2003) Science 302: 462-464. |
| ligase (LplA1). | |
| L. monocytogenes DP-L4017 (10403S with LLO | U.S. Provisional Pat. Appl. Ser. No. 60/490,089 |
| L461T point mutation in hemolysin gene). | filed Jul. 24, 2003. |
| L. monocytogenes EGD. | GenBank Acc. No. AL591824. |
| L. monocytogenes EGD-e. | GenBank Acc. No. NC_003210. ATCC Acc. No. |
| | BAA-679. |
| L. monocytogenes strain EGD, complete genome, | GenBank Acc. No. AL591975 |
| segment 3/12 | |
| L. monocytogenes. | ATCC Nos. 13932; 15313; 19111-19120; 43248-43251; |
| | 51772-51782. |
| L. monocytogenes DP-L4029 deleted in uvrAB. | U.S. Provisional Pat. Appl. Ser. No. 60/541,515 |
| | filed Feb. 2, 2004; U.S. Provisional Pat. |
| | Appl. Ser. No. 60/490,080 filed Jul. 24, 2003. |
| L. monocytogenes DP-L4029 deleted in uvrAB | U.S. Provisional Pat. Appl. Ser. No. 60/541,515 |
| treated with a psoralen. | filed Feb. 2, 2004. |
| L. monocytogenes actA⁻/inlB⁻ double mutant. | Deposited with ATCC on Oct. 3, 2003. Acc. |
| | No. PTA-5562. |
| L. monocytogenes lplA mutant or hly mutant. | U.S. Pat. Applic. No. 20040013690 of Portnoy, et |
| | al. |
| L. monocytogenes DAL/DAT double mutant. | U.S. Pat. Applic. No. 20050048081 of Frankel |
| | and Portnoy. |
| L. monocytogenes str. 4b F2365. | GenBank Acc. No. NC_002973. |
| Listeria ivanovii | ATCC No. 49954 |
| Listeria innocua Clip11262. | GenBank Acc. No. NC_003212; AL592022. |
| Listeria innocua, a naturally occurring hemolytic | Johnson, et al. (2004) Appl. Environ. Microbiol. |
| strain containing the PrfA-regulated virulence | 70: 4256-4266. |
| gene cluster. | |
| Listeria seeligeri. | Howard, et al. (1992) Appl. Eviron. Microbiol. |
| | 58: 709-712. |
| Listeria innocua with L. monocytogenes | Johnson, et al. (2004) Appl. Environ. Microbiol. |
| pathogenicity island genes. | 70: 4256-4266. |
| Listeria innocua with L. monocytogenes | See, e.g., Lingnau, et al. (1995) Infection |
| internalin A gene, e.g., as a plasmid or as a | Immunity 63: 3896-3903; Gaillard, et al. (1991) |
| genomic nucleic acid. | Cell 65: 1127-1141). |
| The present invention encompasses reagents and methods that comprise the above listerial strains, as well as these strains that are modified, e.g., by a plasmid and/or by genomic integration, to contain a nucleic acid encoding one of, or any combination of, the following genes: hly (LLO; listeriolysin); iap (p60); inlA; inlB; inlC; dal (alanine racemase); daaA (dat; D-amino acid aminotransferase); plcA; plcB; actA; or any nucleic acid that mediates growth, | |
| # spread, breakdown of a single walled vesicle, breakdown of a double walled vesicle, binding to a host cell, uptake by a host cell. The present invention is not to be limited by the particular strains disclosed above. | |

In some embodiments, the attenuation of Listeria can be measured in terms of biological effects of the Listeria on a host. The pathogenicity of a strain can be assessed by measurement of the LD₅₀ in mice or other vertebrates. The LD₅₀ is the amount, or dosage, of Listeria injected into vertebrates necessary to cause death in 50% of the vertebrates. The LD₅₀ values can be compared for bacteria having a particular modification (e.g., mutation) versus the bacteria without the particular modification as a measure of the level of attenuation. For example, if the bacterial strain without a particular mutation has an LD₅₀ of 103 bacteria and the bacterial strain having the particular mutation has an LD₅₀ of 105 bacteria, the strain has been attenuated so that is LD₅₀ is increased 100-fold or by 2 log.

In some embodiments, the attenuated Listeria has an LD₅₀ that is at least about 5 times higher, at least about 10 times higher, at least about 100 times higher, at least about 1000 times higher, or at least about 1×10⁴ higher than the LD₅₀ of parental or wildtype Listeria.

As a further example, the degree of attenuation may also be measured qualitatively by other biological effects, such as the extent of tissue pathology or serum liver enzyme levels. Alanine aminotransferase (ALT), aspartate aminotransferase (AST), albumin and bilirubin levels in the serum are determined at a clinical laboratory for mice injected with Listeria (or other bacteria). Comparisons of these effects in mice or other vertebrates can be made for Listeria with and without particular modifications/mutations as a way to assess the attenuation of the Listeria. Attenuation of the Listeria may also be measured by tissue pathology. The amount of Listeria that can be recovered from various tissues of an infected vertebrate, such as the liver, spleen and nervous system, can also be used as a measure of the level of attenuation by comparing these values in vertebrates injected with mutant versus non-mutant Listeria. For instance, the amount of Listeria that can be recovered from infected tissues such as liver or spleen as a function of time can be used as a measure of attenuation by comparing these values in mice injected with mutant vs. non-mutant Listeria.

Accordingly, the attenuation of the Listeria can be measured in terms of bacterial load in particular selected organs in mice known to be targets by wild-type Listeria. For example, the attenuation of the Listeria can be measured by enumerating the colonies (Colony Forming Units; CFU or cfu) arising from plating dilutions of liver or spleen homogenates (homogenized in H₂O+0.2% NP40) on BHI agar media. The liver or spleen cfu can be measured, for example, over a time course following administration of the modified Listeria via any number of routes, including intravenous, intraperitoneal, intramuscular, and subcutaneous. Additionally, the Listeria can be measured and compared to a drug-resistant, wild type Listeria (or any other selected Listeria strain) in the liver and spleen (or any other selected organ) over a time course following administration by the competitive index assay, as described.

Methods of producing mutant Listeria are well known in the art. Bacterial mutations can be achieved through traditional mutagenic methods, such as mutagenic chemicals or radiation followed by selection of mutants. Bacterial mutations can also be achieved by one of skill in the art through recombinant DNA technology. For instance, the method of allelic exchange using the pKSV7 vector described in Camilli et al., Molecular Micro. 8:143-157 (1993) is suitable for use in generating mutants including deletion mutants. (Camilli et al. (1993) is incorporated by reference herein in its entirety.) Alternatively, the gene replacement protocol described in Biswas et al., J. Bacteriol. 175:3628-3635 (1993), can be used. Other similar methods are known to those of ordinary skill in the art.

The construction of a variety of bacterial mutants is described in U.S. patent application Ser. No. 10/883,599, U.S. Patent Publication No. 2004/0197343, and U.S. Patent Publication No. 2004/0228877, each of which is incorporated by reference herein in its entirety.

The degree of attenuation in uptake of the attenuated bacteria by non-phagocytic cells need not be an absolute attenuation in order to provide a safe and effective vaccine. In some embodiments, the degree of attenuation is one that provides for a reduction in toxicity sufficient to prevent or reduce the symptoms of toxicity to levels that are not life threatening.

In some embodiments, the Listeria cannot form colonies, replicate, and/or divide. In some embodiments of the invention, the Listeria is attenuated for proliferation relative to parental or wildtype Listeria.

In some embodiments, the attenuated Listeria is killed, but metabolically active (US Patent Pub. No. 2004/0197343 and Brockstedt, et al., Nat. Med., 11:853-60 (2005), incorporated by reference herein in its entirety).

The Listeria, may, in some embodiments, be attenuated by a nucleic acid targeting compound. In some embodiments, the nucleic-acid targeting compound is a nucleic acid alkylator, such as β-alanine, N-(acridin-9-yl), 2-[bis(2-chloroethyl)amino]ethyl ester. In some embodiments, the nucleic acid targeting compound is activated by irradiation, such as UVA irradiation. In some embodiments, the Listeria is treated with a psoralen compound. For instance, in some embodiments, the bacterium are modified by treatment with a psoralen, such as 4′-(4-amino-2-oxa)butyl-4,5′,8-trimethylpsoralen (“S-59”), and UVA light. In some embodiments, the nucleic acid of the bacterium has been modified by treatment with a psoralen compound and UVA irradiation. Descriptions of methods of modifying bacteria to attenuate them for proliferation using nucleic acid targeting compounds are described in U.S. Patent Pub. No. 2004/0197343 and Brockstedt, et al., Nat. Med., 11:853-60 (2005). In some embodiments, the Listeria is attenuated for DNA repair.

For example, for treatment of Listeria such as ΔactAΔuvrAB L. monocytogenes, in some embodiments, S-59 psoralen can be added to 200 nM in a log-phase culture of (approximately) OD₆₀₀=0.5, followed by inactivation with 6 J/m² of UVA light when the culture reaches an optical density of one. Inactivation conditions are optimized by varying concentrations of S-59, UVA dose, the time of S-59 exposure prior to UVA treatment as well as varying the time of treatment during bacterial growth of the Listeria actA/uvrAB strain. The parental Listeria strain is used as a control. Inactivation of Listeria (log-kill) is determined by the inability of the bacteria to form colonies on BHI (Brain heart infusion) agar plates. In addition, one can confirm the continued metabolic activity and expression of proteins such as LLO in the bacteria in the S-59/UVA inactivated Listeria using ³⁵S-pulse-chase experiments to determine the synthesis and secretion of newly expressed proteins post S-59/UVA inactivation. Expression of LLO using ³⁵S-metabolic labeling can be routinely determined. S-59/UVA inactivated Listeria actA/uvrAB can be incubated for 1 hour in the presence of ³⁵S-Methionine. Expression and/or secretion of proteins such as LLO can be determined of both whole cell lysates, and TCA precipitation of bacterial culture fluids. LLO-specific monoclonal antibodies can be used for immunoprecipitation to verify the continued expression and secretion from recombinant Listeria post inactivation.

In some embodiments, the Listeria attenuated for proliferation are also attenuated for nucleic acid repair and/or are defective with respect to at least one DNA repair enzyme. For instance, in some embodiments, the bacterium in which nucleic acid has been modified by a nucleic acid targeting compound such as a psoralen (combined with UVA treatment) is a uvrAB deletion mutant.

In some embodiments, the proliferation of the Listeria is attenuated by at least about 0.3 log, also at least about 1 log, about 2 log, about 3 log, about 4 log, about 6 log, or at least about 8 log. In another embodiment, the proliferation of the Listeria is attenuated by about 0.3 to >10 log, about 2 to >10 log, about 4 to >10 log, about 6 to >10 log, about 0.3-8 log, about 0.3-6 log, about 0.3-5 log, about 1-5 log, or about 2-5 log. In some embodiments, the expression of LLO by the Listeria is at least about 10%, about 25%, about 50%, about 75%, or at least about 90% of the expression of LLO in non-modified Listeria.

In some embodiments, the Listeria is not an HIV-gag attenuated Listeria described in U.S. Patent Publication No. 2006/0051380, incorporated by reference herein in its entirety. In some embodiments, the Listeria used in the methods described herein do not express an HIV gag polypeptide. In some embodiments, the Listeria used in the methods described herein do not comprise a nucleic acid that encodes an HIV gag polypeptide.

VI. Reagents Administered with an Administered Attenuated Listeria.

The present invention, in certain embodiments, provides reagents for administering in conjunction with an attenuated Listeria. These reagents include biological reagents such as cytokines, dendritic cells, attenuated cancer cell vaccines, and other types of vaccines, small molecule reagents such as 5-fluorouracil, and reagents that modulate regulatory T cells, such as cyclophosphamide or anti-CTLA4 antibody. The reagents can be administered with the Listeria or independently (before or after) the Listeria. For example, the reagent can be administered immediately before (or after) the Listeria, on the same day as, one day before (or after), one week before (or after), one month before (or after), or two months before (or after) the Listeria, and the like.

In some embodiments, in addition to administering the Listeria, the method comprises administering one, or any combination of: a. an agonist or antagonist of a cytokine; b. an inhibitor of a T regulatory cell (Treg); or c. a tumor cell attenuated in growth or replication. In some embodiments, the inhibitor of a Treg used in the methods is cyclophosphamide (CTX).

The present application incorporates by reference U.S. Ser. No. 60/709,700, filed Aug. 19, 2005, in its entirety.

i. Biological reagents. Available biological reagents or macromolecules encompass an agonist or antagonist of a cytokine, a nucleic acid encoding an agonist or antagonist of a cytokine, a cell expressing a cytokine, or an agonistic or antagonistic antibody. Biological reagents include, without limitation, a TH-1 cytokine, a TH-2 cytokine, IL-2, IL-12, FLT3-ligand, GM-CSF, IFNgamma, a cytokine receptor, a soluble cytokine receptor, a chemokine, tumor necrosis factor (TNF), CD40 ligand, or a reagent that stimulates replacement of a proteasome subunit with an immunoproteasome subunit.

The present invention encompasses biological reagents, such cells engineered to express at least one of the following: GM-CSF, IL-2, IL-3, IL-4, IL-12, IL-18, tumor necrosis factor-alpha (TNF-alpha), or inducing protein-10. Other contemplated reagents include agonists of B7-1, B7-2, CD28, CD40 ligand, or OX40 ligand (OX40L), and novel forms engineered to be soluble or engineered to be membrane-bound (see, e.g., Karnbach, et al. (2001) J. Immunol. 167:2569-2576; Greenfield, et al. (1998) Crit. Rev. Immunol. 18:389-418; Parney and Chang (2003) J. Biomed. Sci. 10:37-43; Gri, et al. (2003) J. Immunol. 170:99-106; Chiodoni, et al. (1999) J. Exp. Med. 190:125-133; Enzler, et al. (2003) J. Exp. Med. 197:1213-1219; Soo Hoo, et al. (1999) J. Immunol. 162:7343-7349; Mihalyo, et al. (2004) J. Immunol. 172:5338-5345; Chapoval, et al. (1998) J. Immunol. 161:6977-6984).

Without implying any limitation, the present invention provides the following biologicals. MCP-1, MIP1-alpha, TNF-alpha, and/or interleukin-2, for example, are effective in treating a variety of tumors, including liver tumors (see, e.g., Nakamoto, et al. (2000) Anticancer Res. 20(6A):4087-4096; Kamada, et al. (2000) Cancer Res. 60:6416-6420; Li, et al. (2002) Cancer Res. 62:4023-4028; Yang, et al. (2002) Zhonghua Wai Ke Za Zhi 40:789-791; Hoving, et al. (2005) Cancer Res. 65:4300-4308; Tsuchiyama, et al. (2003) Cancer Gene Ther. 10:260-269; Sakai, et al. (2001) Cancer Gene Ther. 8:695-704).

The present invention, in some aspects, provides reagents and methods encompassing a Flt3-ligand agonist, and an Flt3-ligand agonist in combination with Listeria. Flt3-ligand (Fms-like tyrosine kinase 3 ligand) is a cytokine that can generate an antitumor immune response (see, e.g., Dranoff (2002) Immunol. Revs. 188:147-154; Mach, et al. (2000) Cancer Res. 60:3239-3246; Furumoto, et al. (2004) J. Clin. Invest. 113:774-783; Freedman, et al. (2003) Clin. Cancer Res. 9:5228-5237; Mach, et al. (2000) Cancer Res. 60:3239-3246).

In another embodiment, the present invention contemplates administration of a dendritic cell (DC) that expresses at least one tumor antigen and/or infectious agent antigen. Expression by the DC of an antigen can be mediated by way of, e.g., peptide loading, tumor cell extracts, fusion with tumor cells, transduction with mRNA, or transfection by a vector. Relevant methods are described (see, e.g., Klein, et al. (2000) J. Exp. Med. 191:1699-1708; Conrad and Nestle (2003) Curr. Opin. Mol. Ther. 5:405-412; Gilboa and Vieweg (2004) Immunol. Rev. 199:251-263; Paczesny, et al. (2003) Semin. Cancer Biol. 13:439-447; Westermann, et al. (1998) Gene Ther. 5:264-271).

ii. Small molecule reagents. The methods and reagents of the present invention also encompass small molecule reagents, such as 5-fluorouracil, methotrexate, irinotecan, doxorubicin, prednisone, dolostatin-10 (D10), combretastatin A-4, mitomycin C (MMC), vincristine, colchicines, vinblastine, cyclophosphamide, fungal beta-glucans, and the like (see, e.g., Hurwitz, et al. (2004) New Engl. J. Med. 350:2335-2342; Pelaez, et al. (2001) J. Immunol. 166:6608-6615; Havas, et al. (1990) J. Biol. Response Modifiers 9:194-204; Turk, et al. (2004) J. Exp. Med. 200:771-782; Ghiringhelli, et al. (2004) Eur. J. Immunol. 34:336-344; Andrade-Mena (1994) Int. J. Tissue React. 16:95-103; Chrischilles, et al. (2003) Cancer Control 10:396-403; Hong, et al. (2003) Cancer Res. 63:9023-9031). Also encompassed are compositions that are not molecules, e.g., salts and ions.

Provided are analogues of cyclophosphamide (see, e.g., Jain, et al. (2004) J. Med. Chem. 47:3843-3852; Andersson, et al. (1994) Cancer Res. 54:5394-5400; Borch and Canute (1991) J. Med. Chem. 34:3044-3052; Ludeman, et al. (1979) J. Med. Chem. 22:151-158; Zon (1982) Prog. Med. Chem. 19:205-246).

Also embraced by the invention are small molecule reagents that stimulate innate immune response, e.g., CpG oligonucleotides, imiquimod, and alphaGalCer. CpG oligonucleotides mediate immune response via TLR9 (see, e.g., Chagnon, et al. (2005) Clin. Cancer Res. 11:1302-1311; Speiser, et al. (2005) J. Clin. Invest. Feb. 3 (epub ahead of print); Mason, et al. (2005) Clin. Cancer Res. 11:361-369; Suzuki, et al. (2004) Cancer Res. 64:8754-8760; Taniguchi, et al. (2003) Annu. Rev. Immunol. 21:483-513; Takeda, et al. (2003) Annu. Rev. Immunol. 21:335-376; Metelitsa, et al. (2001) J. Immunol. 167:3114-3122).

Other useful small molecule reagents include those derived from bacterial peptidoglycan, such as certain NOD1 ligands and/or NOD2 ligands, such as diaminopimelate-containing muropeptides (see, e.g., McCaffrey, et al. (2004) Proc. Natl. Acad. Sci. USA 101:11386-11391; Royet and Reighhart (2003) Trends Cell Biol. 13:610-614; Chamaillard, et al. (2003) Nature Immunol. 4:702-707; Inohara and Nunez (2003) Nature Rev. Immunol. 3:371-382; Inohara, et al. (2004) Annu. Rev. Biochem. Nov. 19 [epub ahead of print]).

iii. Regulatory T cells. The invention includes reagents and methods for modulating activity of T regulatory cells (Tregs; suppressor T cells). Attenuation or inhibition of Treg cell activity can enhance the immune system's killing of tumor cells. A number of reagents have been identified that inhibit Treg cell activity. These reagents include, e.g., cyclophosphamide (a.k.a. Cytoxan®; CTX), anti-CD25 antibody, modulators of GITR-L or GITR, a modulator of Forkhead-box transcription factor (Fox), a modulator of LAG-3, anti-IL-2R, and anti-CTLA4 (see, e.g., Pardoll (2003) Annu. Rev. Immunol. 21:807-839; Ercolini, et al. (2005) J. Exp. Med. 201:1591-1602; Haeryfar, et al. (2005) J. Immunol. 174:3344-3351; Ercolini, et al. (2005) J. Exp. Med. 201:1591-1602; Mihalyo, et al. (2004) J. Immunol. 172:5338-5345; Stephens, et al. (2004) J. Immunol. 173:5008-5020; Schiavoni, et al. (2000) Blood 95:2024-2030; Calmels, et al. (2004) Cancer Gene Ther. Oct. 8 (epub ahead of print); Mincheff, et al. (2004) Cancer Gene Ther. September 17 [epub ahead of print]; Muriglan, et al. (2004) J. Exp. Med. 200:149-157; Stephens, et al. (2004) J. Immunol. 173:5008-5020; Coffer and Burgering (2004) Nat. Rev. Immunol. 4:889-899; Kalinichenko, et al. (2004) Genes Dev. 18:830-850; Cobbold, et al. (2004) J. Immunol. 172:6003-6010; Huang, et al. (2004) Immunity 21:503-513). CTX shows a bimodal effect on the immune system, where low doses of CTX inhibit Tregs (see, e.g., Lutsiak, et al. (2005) Blood 105:2862-2868).

CTLA4-blocking agents, such as anti-CTLA4 blocking antibodies, can enhance immune response, e.g., to cancers (see, e.g., Zubairi, et al. (2004) Eur. J. Immunol. 34:1433-1440; Espenschied, et al. (2003) J. Immunol. 170:3401-3407; Davila, et al. (2003) Cancer Res. 63:3281-3288; Hodi, et al. (2003) Proc. Natl. Acad. Sci. USA 100:4712-4717). Where the present invention uses anti-CTLA4 antibodies, and the like, the invention is not necessarily limited to use for inhibiting Tregs, and also does not necessarily always encompass inhibition of Tregs.

Lymphocyte activation gene-3 (LAG-3) blocking agents, such as anti-LAG-3 antibodies or soluble LAG-3 (e.g., LAG-3 Ig), can enhance immune response to proliferative disorders. Anti-LAG-3 antibodies reduce the activity of Tregs (see, e.g., Huang, et al. (2004) Immunity 21:503-513; Triebel (2003) Trends Immunol. 24:619-622; Workman and Vignali (2003) Eur. J. Immunol. 33:970-979; Cappello, et al. (2003) Cancer Res. 63:2518-2525; Workman, et al. (2004) J. Immunol. 172:5450-5455; Macon-Lemaitre and Triebel (2005) Immunology 115:170-178).

iv. Vaccines. Vaccines comprising a tumor antigen, a nucleic acid encoding a tumor antigen, a vector comprising a nucleic acid encoding a tumor antigen, a cell comprising a tumor antigen, a tumor cell, or an attenuated tumor cell, are encompassed by the invention. Provided are reagents derived from a nucleic acid encoding a tumor antigen, e.g., a codon optimized nucleic acid, or a nucleic acid encoding two or more different tumor antigens, or a nucleic acid expressing rearranged epitopes of a tumor antigen, e.g., where the natural order of epitopes is ABCD and the engineered order is ADBC, or a nucleic acid encoding a fusion protein comprising at least two different tumor antigens.

Vaccines comprising a tumor cell, an attenuated tumor cell, or a recombinant tumor cell engineered to express a cytokine or other immune modulating agent, are provided by the present invention. For example, a tumor cell can be engineered to express an agent that modulates immune response, e.g., GM-CSF, IL-2, IL-4, or IFNgamma (see, e.g., U.S. Pat. Nos. 6,033,674 and 6,350,445 issued to Jaffee, et al.; Golumbek, et al. (1991) Science 254:713-716; Ewend, et al. (2000) J. Immunother. 23:438-448; Zhou, et al. (2005) Cancer Res. 65:1079-1088; Porgador, et al. (1993) J. Immunol. 150:1458-1470; Poloso, et al. (2001) Front. Biosci. 6:D760-D775). The vaccine can be administered by a gel matrix (see, e.g., Salem, et al. (2004) J. Immunol. 172:5159-5167).

The present invention, in some embodiments, also provides a vaccine comprising a dendritic cell (or other APC) engineered to express a tumor antigen (see, e.g., Avigan (1999) Blood Rev. 13:51-64; Kirk and Mule (2000) Hum. Gene Ther. 11:797-806). Also provided are, e.g., synthetic peptides, purified antigens, oligosaccharides, and tumor cell lysates, as a source of tumor antigen (see, e.g., Lewis, et al. (2003) Int. Rev. Immunol. 22:81-112; Razzaque, et al. (2000) Vaccine 19:644-647; Meng and Butterfield (2002) Pharm. Res. 19:926-932; Le Poole, et al. (2002) Curr. Opin. Oncol. 14:641-648). Moreover, the present invention provides a heat shock protein, where the heat shock protein elicits tumor-specific immunity (see, e.g., Udono, et al. (1994) Proc. Natl. Acad. Sci. USA 91:3077-3081; Wang, et al. (2000) Immunol. Invest. 29:131-137).

The invention includes at least one antigen, or nucleic acid encoding at least one antigen, for use in a vaccine (see, e.g., Table 3). In another aspect, the present invention does not provide any nucleic acid encoding a tumor antigen, does not provide any tumor antigen, does not provide any nucleic acid encoding an infectious agent, and/or does not provide any infectious agent antigen. In another aspect, the present invention does not provide any nucleic acid encoding a tumor antigen, or does not provided any tumor antigen, or does not provide any nucleic acid encoding an infectious agent antigen, or does not provide any infectious agent antigen, or any combination thereof. The antigen can be provided or administered by way of, for example, a composition comprising at least one isolated protein, a composition comprising at least one isolated protein fragment, a nucleic acid vaccine, or a virus-based vaccine, and the like (see, e.g., Polo and Dubensky (2002) Drug Discovery Today 7:719-727; Cheng, et al. (2005) Vaccine 23:3864-3874; Kim, et al. (2005) Hum. Gene Ther. 16:26-34).

The Listeria of the present invention can be engineered by any of a number of methods that effect attenuation. The Listeria can also be engineered to express a selection marker. Methods are described (see, e.g., Camilli, et al. (1993) Mol. Microbiol. 8:143-157; Camilli (1992) Genetic analysis of Listeria monocytogenes Determinants of Pathogenesis, Univ. of Pennsylvania, Doctoral thesis; Thompson, et al. (1998) Infect. Immunity 66:3552-3561; Skoble, et al. (2000) J. Cell Biol. 150:527-537; Smith and Youngman (1992) Biochimie 74:705-711; Lei, et al. (2001) J. Bact. 183:1133-1139; L1 and Kathariou (2003) Appl. Environ. Microbiol. 69:3020-3023; Lauer, et al. (2002) J. Bacteriol. 184:4177-4186).

Alternatively, or in addition, the vaccine can be administered as a nucleic acid vaccine, liposome, soluble antigen, particulate antigen, colloidal antigen, conjugated antigen, an engineered tumor cell, or an attenuated tumor cell. The vaccine can take the form of a nucleic acid vaccine, liposome, soluble antigen, particulate antigen, colloidal antigen, conjugated antigen, an engineered tumor cell, or an attenuated tumor cell.

The list of methods of administration, are not intended to be limiting to the present invention.

TABLE 3
Antigens and nucleic acids encoding antigens.

| | |
|---|---|
| Antigen | Reference |

Tumor antigen

| | |
|---|---|
| Mesothelin | GenBank Acc. No. NM_005823; U40434; NM_013404; BC003512 (see |
| | also, e.g., Hassan, et al. (2004) Clin. Cancer Res. 10: 3937-3942; |
| | Muminova, et al. (2004) BMC Cancer 4:19; Iacobuzio-Donahue, et al. |
| | (2003) Cancer Res. 63: 8614-8622). |
| Prostate acid phosphatase | Small, et al. (2000) J. Clin. Oncol. 18: 3894-3903; Altwein and Luboldt |
| (PAP); prostate-specific | (1999) Urol. Int. 63: 62-71; Chan, et al. (1999) Prostate 41: 99-109; Ito, et |
| antigen (PSA); PSM; | al. (2005) Cancer 103: 242-250; Schmittgen, et al. (2003) Int. J. Cancer |
| PSMA. | 107: 323-329; Millon, et al. (1999) Eur. Urol. 36: 278-285. |
| Proteinase 3. | GenBank Acc. No. X55668. |
| Cancer-testis antigens, | GenBank Acc. No. NM_001327 (NY-ESO-1) (see also, e.g., Li, et al. |
| e.g., NY-ESO-1; SCP-1; | (2005) Clin. Cancer Res. 11: 1809-1814; Chen, et al. (2004) Proc. Natl. |
| SSX-1; SSX-2; SSX-4; | Acad. Sci. USA. 101(25): 9363-9368; Kubuschok, et al. (2004) Int. J. |
| GAGE, CT7; CT8; CT10; | Cancer. 109: 568-575; Scanlan, et al. (2004) Cancer Immun. 4:1; Scanlan, |
| MAGE-1; MAGE-2; | et al. (2002) Cancer Res. 62: 4041-4047; Scanlan, et al. (2000) Cancer |
| MAGE-3; MAGE-4; | Lett. 150: 155-164; Dalerba, et al. (2001) Int. J. Cancer 93: 85-90; Ries, et |
| MAGE-6; LAGE-1. | al. (2005) Int. J. Oncol. 26: 817-824. |
| MAGE-A1, MAGE-A2; | Otte, et al. (2001) Cancer Res. 61: 6682-6687; Lee, et al. (2003) Proc. Natl. |
| MAGE-A3; MAGE-A4; | Acad. Sci. USA 100: 2651-2656; Sarcevic, et al. (2003) Oncology 64: 443-449; |
| MAGE-A6; MAGE-A9; | Lin, et al. (2004) Clin. Cancer Res. 10: 5708-5716. |
| MAGE-A10; | |
| MAGE-A12; GAGE-3/6; | |
| NT-SAR-35; BAGE; | |
| CA125. | |
| GAGE-1; GAGE-2; | De Backer, et al. (1999) Cancer Res. 59: 3157-3165; Scarcella, et al. |
| GAGE-3; GAGE-4; | (1999) Clin. Cancer Res. 5: 335-341. |
| GAGE-5; GAGE-6; | |
| GAGE-7; GAGE-8; | |
| GAGE-65; GAGE-11; | |
| GAGE-13; GAGE-7B. | |
| HIP1R; LMNA; | Scanlan, et al. (2002) Cancer Res. 62: 4041-4047. |
| KIAA1416; Seb4D; | |
| KNSL6; TRIP4; MBD2; | |
| HCAC5; MAGEA3. | |
| Colon cancer associated | Scanlan, et al. (2002) Cancer Res. 62: 4041-4047. |
| antigens, e.g., NY-CO-8; | |
| NY-CO-9; NY-CO-13; | |
| NY-CO-16; NY-CO-20; | |
| NY-CO-38; NY-CO-45; | |
| NY-CO-9/HDAC5; | |
| NY-CO-41/MBD2; | |
| NY-CO-42/TRIP4; | |
| NY-CO-95/KIAA1416; | |
| KNSL6; seb4D. | |
| MUM-1 (melanoma | Gueguen, et al. (1998) J. Immunol. 160: 6188-6194; Hirose, et al. (2005) |
| ubiquitous mutated); | Int. J. Hematol. 81: 48-57; Baurain, et al. (2000) J. Immunol. 164: 6057-6066; |
| MUM-2; MUM-2 Arg- | Chiari, et al. (1999) Cancer Res. 59: 5785-5792. |
| Gly mutation; MUM-3. | |
| NY-REN series of renal | Scanlan, et al. (2002) Cancer Res. 62: 4041-4047; Scanlan, et al. (1999) |
| cancer antigens. | Cancer Res. 83: 456-464. |
| NY-BR series of breast | Scanlan, et al. (2002) Cancer Res. 62: 4041-4047; Scanlan, et al. (2001) |
| cancer antigens, e.g., | Cancer Immunity 1:4. |
| NY-BR-62; NY-BR-75; | |
| NY-BR-85; NY-BR-62; | |
| NY-BR-85. | |
| BRCA-1; BRCA-2. | Stolier, et al. (2004) Breast J. 10: 475-480; Nicoletto, et al. (2001) Cancer |
| | Treat Rev. 27: 295-304. |
| Ras, e.g., wild type ras, | GenBank Acc. No. P01112; P01116; M54969; M54968; P01111; P01112; |
| ras with mutations at | K00654. |
| codon 12, 13, 59, or 61, | |
| e.g., mutations G12C; | |
| G12D; G12R; G12S; | |
| G12V; G13D; A59T; | |
| Q61H. K-RAS; H-RAS; | |
| N-RAS. | |
| Melanoma antigens, | GenBank Acc. No. NM_206956; NM_206955; NM_206954; |
| including HST-2 | NM_206953; NM_006115; NM_005367; NM_004988; AY148486; |
| melanoma cell antigens. | U10340; U10339; M77481. See, e g., Suzuki, et al. (1999) J. Immunol. |
| | 163: 2783-2791. |
| Survivin | GenBank Acc. No. AB028869; U75285 (see also, e.g., Tsuruma, et al. |
| | (2004) J. Translational Med. 2:19 (11 pages); Pisarev, et al. (2003) Clin. |
| | Cancer Res. 9: 6523-6533; Siegel, et al. (2003) Br. J. Haematol. 122: 911-914; |
| | Andersen, et al. (2002) Histol. Histopathol. 17: 669-675). |
| MDM-2 | NM_002392; NM_006878 (see also, e.g., Mayo, et al. (1997) Cancer Res. |
| | 57: 5013-5016; Demidenko and Blagosklonny (2004) Cancer Res. |
| | 64: 3653-3660). |
| GAGE/PAGE family, | Brinkmann, et al. (1999) Cancer Res. 59: 1445-1448. |
| e.g., PAGE-1; PAGE-2; | |
| PAGE-3; PAGE-4; | |
| XAGE-1; XAGE-2; | |
| XAGE-3. | |
| MAGE-A, B, C, and D | Lucas, et al. (2000) Int. J. Cancer 87: 55-60; Scanlan, et al. (2001) Cancer |
| families. MAGE-B5; | Immun. 1:4. |
| MAGE-B6; MAGE-C2; | |
| MAGE-C3; MAGE-3; | |
| MAGE-6. | |
| Carcinoembryonic | GenBank Acc. No. M29540; E03352; X98311; M17303 (see also, e.g., |
| antigen (CEA), CAP1-6D | Zaremba (1997) Cancer Res. 57: 4570-4577; Sarobe, et al. (2004) Curr. |
| enhancer agonist peptide. | Cancer Drug Targets 4: 443-454; Tsang, et al. (1997) Clin. Cancer Res. |
| | 3: 2439-2449; Fong, et al. (2001) Proc. Natl. Acad. Sci. USA 98: 8809-8814). |
| HER-2/neu. | Disis, et al. (2004) J. Clin. Immunol. 24: 571-578; Disis and Cheever |
| | (1997) Adv. Cancer Res. 71: 343-371. |
| Tyrosinase-related | GenBank Acc. No. NM_001922. (see also, e.g., Bronte, et al. (2000) |
| proteins 1 and 2 (TRP-1 | Cancer Res. 60: 253-258). |
| and TRP-2). | |
| gp100/pmel-17. | GenBank Acc. Nos. AH003567; U31798; U31799; U31807; U31799 (see |
| | also, e.g., Bronte, et al. (2000) Cancer Res. 60: 253-258). |
| TARP. | See, e.g., Clifton, et al. (2004) Proc. Natl. Acad. Sci. USA 101: 10166-10171; |
| | Virok, et al. (2005) Infection Immunity 73: 1939-1946. |
| Tyrosinase-related | GenBank Acc. No. NM_001922. (see also, e.g., Bronte, et al. (2000) |
| proteins 1 and 2 (TRP-1 | Cancer Res. 60: 253-258). |
| and TRP-2). | |
| Melanocortin 1 receptor | Salazar-Onfray, et al. (1997) Cancer Res. 57: 4348-4355; Reynolds, et al. |
| (MC1R); MAGE-3; | (1998) J. Immunol. 161: 6970-6976; Chang, et al. (2002) Clin. Cancer Res. |
| gp100; tyrosinase; | 8: 1021-1032. |
| dopachrome tautomerase | |
| (TRP-2); MART-1. | |
| MUC-1; MUC-2. | See, e.g., Davies, et al. (1994) Cancer Lett. 82: 179-184; Gambus, et al. |
| | (1995) Int. J. Cancer 60: 146-148; McCool, et al. (1999) Biochem. J. |
| | 341: 593-600. |

Polyomavirus, including SV40

| | |
|---|---|
| Polyomavirus, | Engels, et al. (2004) J. Infect. Dis. 190: 2065-2069; Vilchez and |
| including simian | Butel (2004) Clin. Microbiol. Rev. 17: 495-508; Shivapurkar, et al. |
| virus 40 (SV40), JC | (2004) Cancer Res. 64: 3757-3760; Carbone, et al. (2003) Oncogene |
| virus (JCV) and BK | 2: 5173-5180; Barbanti-Brodano, et al. (2004) Virology 318: 1-9. |
| virus (BKV). | (SV40 complete genome in, e.g., GenBank Acc. Nos. NC_001669; |
| | AF168994; AY271817; AY271816; AY120890; AF345344; |
| | AF332562). |

Human papillomavirus

| | |
|---|---|
| Human papillomavirus. | Complete genome (see, e.g., GenBank Acc. Nos. AY686584; |
| | AY686583; AY686582; NC_006169; NC_006168; NC_006164; |
| | NC_001355; NC_001349; NC_005351; NC_001596). |
| Human papillomavirus | See, e.g., Trimble, et al. (2003) Vaccine 21: 4036-4042; Kim, et al. |
| type-16 E7 (HPV 16 E7). | (2004) Gene Ther. 11: 1011-1018; Simon, et al. (2003) Eur. J. |
| | Obstet. Gynecol. Reprod. Biol. 109: 219-223. |

Hepatitis viruses

| Hepatitis B | Complete genome (see, e.g., GenBank Acc. Nos. AB214516; NC_003977; |
|---|---|
| | AB205192; AB205191; AB205190; AJ748098; AB198079; AB198078; |
| | AB198076; AB074756). |
| Hepatitis C | Complete genome (see, e.g., GenBank Acc. Nos. NC_004102; AJ238800; |
| | AJ238799; AJ132997; AJ132996; AJ000009; D84263). |
| In addition to providing a Listeria that does not contain a nucleic acid encoding a tumor antigen, infectious agent antigen, or proliferative disorder antigen, the present invention encompasses reagents and methods for administering, a protein, a protein fragment, a protein complex, a DNA vaccine, a virus-based vaccine, or an engineered tumor cell, of the above-disclosed antigens. The present invention encompasses nucleic acids encoding mutants, | |
| # muteins, splice variants, fragments, truncated variants, soluble variants, extracellular domains, intracellular domains, mature sequences, and the like, of the disclosed antigens. Provided are nucleic acids encoding epitopes, oligo- and polypeptides of these antigens. Also provided are codon optimized embodiments, i.e., optimized for expression in Listeria. The cited references and the nucleic acids, peptides, and polypeptides disclosed therein, are all incorporated herein | |
| # by reference in their entirety. The list of antigens and their nucleic acids. The list of methods of administration, are not intended to be limiting to the present invention. | |

VII. Therapeutic and Other Compositions.

A variety of compositions (e.g., pharmaceutical compositions, vaccines, immunogenic compositions, etc.) comprising the attenuated Listeria and useful in the methods of the invention are provided herein. The attenuated Listeria, vaccines, small molecules, biological reagents, and adjuvants that are provided herein can be administered to a host, either alone or in combination with a pharmaceutically acceptable excipient, in an amount sufficient to induce an appropriate immune response to an immune disorder, proliferative disorder, cancer, cancerous disorder, or infectious disorder. The immune response can comprise, without limitation, a specific response, non-specific response, a specific and non-specific response, innate response, adaptive immunity, primary immune response, secondary immune response, memory immune response, immune cell activation, immune cell proliferation, and immune cell differentiation.

A “pharmaceutically acceptable excipient” or “diagnostically acceptable excipient” is meant to include, but is not limited to, sterile distilled water, saline, phosphate buffered solutions, amino acid-based buffers, or bicarbonate buffered solutions. An excipient selected and the amount of excipient used will depend upon the mode of administration. Administration may be oral, intravenous, subcutaneous, dermal, intradermal, intramuscular, parenteral, intraorgan, intralesional, intranasal, inhalation, intraocular, intramuscular, intravascular, intrarectal, intraperitoneal, or any one of a variety of well-known routes of administration. In some embodiments, the administration is mucosal. The administration can comprise an injection, infusion, or a combination thereof. In some embodiments, the administration is not oral. In some embodiments, the administration is intravenous.

The invention provides, in certain embodiments, pharmaceutical compositions comprising the attenuated Listeria and a pharmaceutically acceptable excipient. In some embodiments, pharmaceutical compositions comprising the attenuated Listeria comprise an adjuvant.

In some embodiments, the Listeria is administered in a composition that is at least about 90%, at least about 95, or at least 99% free of other types of bacteria.

The Listeria of the present invention can be stored, e.g., frozen, lyophilized, as a suspension, as a cell paste, or complexed with a solid matrix or gel matrix.

An effective amount for a particular patient may vary depending on factors such as the condition being treated, the overall health of the patient, the method route and dose of administration and the severity of side affects. An effective amount for a particular patient may vary depending on factors such as the condition being treated, the overall health of the patient, the method route and dose of administration and the severity of side affects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

The Listeria of the present invention, in some embodiments, can be administered in a dose, or dosages, where each dose comprises at least 1000 Listeria cells/kg body weight; normally at least 10,000 cells; more normally at least 100,000 cells; most normally at least 1 million cells; often at least 10 million cells; more often at least 100 million cells; most often at least 1 billion cells; usually at least 10 billion cells; more usually at least 100 billion cells; and most usually at least 1 trillion Listeria cells/kg body weight. The present invention provides the above doses where the units of Listeria administration is colony forming units (CFU), the equivalent of CFU prior to psoralen-treatment, or where the units are number of Listeria cells. In some embodiments, the effective amount of attenuated Listeria that is measured comprises at least about 1×10³ CFU/kg or at least about 1×10³ Listeria cells/kg. In some embodiments, the effective amount of attenuated Listeria that is measured comprises at least about 1×10⁵ CFU/kg or at least about 1×10⁵ Listeria cells/kg. In certain embodiments, the effective amount of attenuated Listeria that is measured comprises at least about 1×10⁶ CFU/kg or at least about 1×10⁶ Listeria cells/kg. In some embodiments, the effective amount of attenuated Listeria that is measured comprises at least about 1×10⁷ CFU/kg or at least about 1×10⁷ Listeria cells/kg. In some further embodiments, the effective amount of attenuated Listeria that is measured comprises at least about 1×10⁸ CFU/kg or at least about 1×10⁸ Listeria cells/kg.

The Listeria of the present invention, in certain embodiments, can be administered in a dose, or dosages, where each dose comprises between 107 and 108 Listeria per 70 kg body weight (or per 1.7 square meters surface area; or per 1.5 kg liver weight); 2×10⁷ and 2×10⁸ Listeria per 70 kg body weight (or per 1.7 square meters surface area; or per 1.5 kg liver weight); 5×10⁷ and 5×10⁸ Listeria per 70 kg body weight (or per 1.7 square meters surface area; or per 1.5 kg liver weight); 108 and 109 Listeria per 70 kg body weight (or per 1.7 square meters surface area; or per 1.5 kg liver weight); between 2.0×10⁸ and 2.0×10⁹ Listeria per 70 kg (or per 1.7 square meters surface area, or per 1.5 kg liver weight); between 5.0×10⁸ to 5.0×10⁹ Listeria per 70 kg (or per 1.7 square meters surface area, or per 1.5 kg liver weight); between 10⁹ and 1010 Listeria per 70 kg (or per 1.7 square meters surface area, or per 1.5 kg liver weight); between 2×10⁹ and 2×10¹⁰ Listeria per 70 kg (or per 1.7 square meters surface area, or per 1.5 kg liver weight); between 5×10⁹ and 5×10¹⁰ Listeria per 70 kg (or per 1.7 square meters surface area, or per 1.5 kg liver weight); between 1101 and 1012 Listeria per 70 kg (or per 1.7 square meters surface area, or per 1.5 kg liver weight); between 2×10¹¹ and 2×10¹² Listeria per 70 kg (or per 1.7 square meters surface area, or per 1.5 kg liver weight); between 5×10¹¹ and 5×10¹² Listeria per 70 kg (or per 1.7 square meters surface area, or per 1.5 kg liver weight); between 10¹² and 1013 Listeria per 70 kg (or per 1.7 square meters surface area); between 2×10¹² and 2×10¹³ Listeria per 70 kg (or per 1.7 square meters surface area, or per 1.5 kg liver weight); between 5×10¹² and 5×10¹³ Listeria per 70 kg (or per 1.7 square meters surface area, or per 1.5 kg liver weight); between 10¹³ and 1014 Listeria per 70 kg (or per 1.7 square meters surface area, or per 1.5 kg liver weight); between 2×10¹³ and 2×10¹⁴ Listeria per 70 kg (or per 1.7 square meters surface area, or per 1.5 kg liver weight); 5×10¹³ and 5×10¹⁴ Listeria per 70 kg (or per 1.7 square meters surface area, or per 1.5 kg liver weight); between 10¹⁴ and 10¹⁵ Listeria per 70 kg (or per 1.7 square meters surface area, or per 1.5 kg liver weight); between 2×10¹⁴ and 2×10⁵ Listeria per 70 kg (or per 1.7 square meters surface area, or per 1.5 kg liver weight); between 5×10¹⁴ and 5×10¹⁵ Listeria per 70 kg (or per 1.7 square meters surface area, or per 1.5 kg liver weight); between 10¹⁵ and 10¹⁶ Listeria per 70 kg (or per 1.7 square meters surface area, or per 1.5 kg liver weight); between 2×10¹⁵ and 2×10¹⁶ Listeria per 70 kg (or per 1.7 square meters surface area, or per 1.5 kg liver weight); and between 5×10¹⁵ and 5×10¹⁶ Listeria per 70 kg (or per 1.7 square meters surface area, or per 1.5 kg liver weight). The number of Listeria can be determined by, e.g., counting individual bacteria under a microscope or by counting colony forming units (CFUs). The mouse liver, at the time of administering the Listeria of the present invention, weighs about 1.5 grams. Human liver weighs about 1.5 kilograms.

In some embodiments, the attenuated Listeria is administered to the mammal in two or more doses. In some embodiments, the attenuated Listeria is administered to the mammal in three or more doses. In some embodiments, the attenuated Listeria is administered to the mammal in four or more, five or more, or six or more doses. The Listeria used in the later dose(s) may or may not be identical to the Listeria in the earlier dose(s).

In some embodiments, the attenuated Listeria is administered in multiple doses. An effective amount may be administered to a mammal in the form of multiple doses of the Listeria or multiple doses of an effective amount of the Listeria may be administered. In those methods in which a plurality of doses of the Listeria are administered, the second dose may be administered at least about 5 minutes after the first dose, at least about 15 minutes after the first dose, at least about one hour after the first dose, at least about 6 hours after the first dose, at least about 12 hours after the first dose, at least about 24 hours after the first dose, at least about 3 days after the first dose, at least about 1 week after the first dose, at least about two weeks after the first dose, at least about one month after the first dose or at least about 6 months after the first dose. Likewise, the third dose may be administered at least about 5 minutes after the second dose, at least about 15 minutes after the second dose, at least about one hour after the second dose, at least about 6 hours after the second dose, at least about 12 hours after the second dose, at least about 24 hours after the second dose, at least about 3 days after the second dose, at least about 1 week after the second dose, at least about two weeks after the second dose, at least about one month after the second dose or at least about 6 months after the second dose. In some embodiments of the methods described herein, the multiple doses of the attenuated Listeria is all given within a time period of about one hour, about 1 day, about one week, about two weeks, about one month, about three months, about six months, about 1 year, about 5 years, or about 10 years.

Also provided is one or more of the above doses, where the dose is administered by way of one injection every day, one injection every two days, one injection every three days, one injection every four days, one injection every five days, one injection every six days, or one injection every seven days, where the injection schedule is maintained for, e.g., one day only, two days, three days, four days, five days, six days, seven days, two weeks, three weeks, four weeks, five weeks, or longer. The invention also embraces combinations of the above doses and schedules, e.g., a relatively large initial dose of Listeria, followed by relatively small subsequent doses of Listeria.

A dosing schedule of, for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

The present invention encompasses a method of administering Listeria that is oral. Also provided is a method of administering Listeria that is intravenous. Moreover, what is provided is a method of administering Listeria that is intramuscular. The invention supplies a Listeria bacterium, or culture or suspension of Listeria bacteria, prepared by growing in a medium that is meat based, or that contains polypeptides derived from a meat or animal product. Also supplied by the present invention is a Listeria bacterium, or culture or suspension of Listeria bacteria, prepared by growing in a medium that does not contain meat or animal products, prepared by growing on a medium that contains vegetable polypeptides, prepared by growing on a medium that is not based on yeast products, or prepared by growing on a medium that contains yeast polypeptides.

The present invention encompasses a method of administering Listeria that is not oral. Also provided is a method of administering Listeria that is not intravenous. Moreover, what is provided is a method of administering Listeria that is not intramuscular. The invention supplies a Listeria bacterium, or culture or suspension of Listeria bacteria, prepared by growing in a medium that is not meat based, or that does not contain polypeptides derived from a meat or animal product. Also supplied by the present invention is a Listeria bacterium, or culture or suspension of Listeria bacteria, prepared by growing in a medium based on vegetable products, that contains vegetable polypeptides, that is based on yeast products, or that contains yeast polypeptides.

In some embodiments, the methods of the present invention do not utilize, and specifically exclude, the method of administration of a Listeria bacterium disclosed by U.S. Publication No. 2006/0051380.

Methods for co-administration or treatment with an additional therapeutic agent, e.g., a cytokine, chemotherapeutic agent, antibiotic, or radiation, are well known in the art (Hardman, et al. (eds.) (2001) Goodman and Gilman's The Pharmacological Basis of Therapeutics, 10^{th} ed., McGraw-Hill, New York, N.Y.; Poole and Peterson (eds.) (2001) Pharmacotherapeutics for Advanced Practice: A Practical Approach, Lippincott, Williams & Wilkins, Phila., PA; Chabner and Longo (eds.) (2001) Cancer Chemotherapy and Biotherapy, Lippincott, Williams & Wilkins, Phila., PA).

Where an administered antibody, cytokine, or other therapeutic agent produces toxicity, an appropriate dose can be one where the therapeutic effect outweighs the toxic effect. Generally, an optimal dosage of the present invention is one that maximizes therapeutic effect, while limiting any toxic effect to a level that does not threaten the life of the patient or reduce the efficacy of the therapeutic agent. Signs of toxic effect, or anti-therapeutic effect include, without limitation, e.g., anti-idiotypic response, immune response to a therapeutic antibody, allergic reaction, hematologic and platelet toxicity, elevations of aminotransferases, alkaline phosphatase, creatine kinase, neurotoxicity, nausea, and vomiting (see, e.g., Huang, et al. (1990) Clin. Chem. 36:431-434).

An effective amount of a therapeutic agent is one that will decrease or ameliorate the symptoms normally by at least 10%, more normally by at least 20%, most normally by at least 30%, typically by at least 40%, more typically by at least 50%, most typically by at least 60%, often by at least 70%, more often by at least 80%, and most often by at least 90%, conventionally by at least 95%, more conventionally by at least 99%, and most conventionally by at least 99.9%.

The reagents and methods of the present invention optionally provide a vaccine comprising only one vaccination; or comprising a first vaccination; or comprising at least one booster vaccination; at least two booster vaccinations; or at least three booster vaccinations. Guidance in parameters for booster vaccinations is available (see, e.g., Marth (1997) Biologicals 25:199-203; Ramsay, et al. (1997) Immunol. Cell Biol. 75:382-388; Gherardi, et al. (2001) Histol. Histopathol. 16:655-667; Leroux-Roels, et al. (2001) Acta Clin. Belg. 56:209-219; Greiner, et al. (2002) Cancer Res. 62:6944-6951; Smith, et al. (2003) J. Med. Virol. 70:Suppl. 1:S38-S41; Sepulveda-Amor, et al. (2002) Vaccine 20:2790-2795).

Provided is a first reagent that comprises a Listeria bacterium or Listeria vaccine, and a second reagent that comprises, e.g., a cytokine, a small molecule such as cyclophosphamide or methotrexate, or a vaccine, such as an attenuated tumor cell or attenuated tumor cell expressing a cytokine. Provided are the following methods of administration of the first reagent and the second reagent.

The Listeria and the second reagent can be administered concomitantly, that is, where the administering for each of these reagents can occur at time intervals that partially or fully overlap each other. The Listeria and second reagent can be administered during time intervals that do not overlap each other. For example, the first reagent can be administered within the time frame of t=0 to 1 hours, while the second reagent can be administered within the time frame of t=1 to 2 hours. Also, the first reagent can be administered within the time frame of t=0 to 1 hours, while the second reagent can be administered somewhere within the time frame of t=2-3 hours, t=3-4 hours, t=4-5 hours, t=5-6 hours, t=6-7 hours, t=7-8 hours, t=8-9 hours, t=9-10 hours, and the like. Moreover, the second reagent can be administered somewhere in the time frame of t=minus 2-3 hours, t=minus 3-4 hours, t=minus 4-5 hours, t=5-6 minus hours, t=minus 6-7 hours, t=minus 7-8 hours, t=minus 8-9 hours, t=minus 9-10 hours, and the like.

To provide another example, the first reagent can be administered within the time frame of t=0 to 1 days, while the second reagent can be administered within the time frame of t=1 to 2 days. Also, the first reagent can be administered within the time frame of t=0 to 1 days, while the second reagent can be administered somewhere within the time frame of t=2-3 days, t=3-4 days, t=4-5 days, t=5-6 days, t=6-7 days, t=7-8 days, t=8-9 days, t=9-10 days, and the like. Moreover, the second reagent can be administered somewhere in the time from of t=minus 2-3 days, t=minus 3-4 days, t=minus 4-5 days, t=minus 5-6 days, t=minus 6-7 days, t=minus 7-8 days, t=minus 8-9 days, t=minus 9-10 days, and the like.

In another aspect, administration of the Listeria can begin at t=0 hours, where the administration results in a peak (or maximal plateau) in plasma concentration of the Listeria, and where administration of the second reagent is initiated at about the time that the concentration of plasma Listeria reaches said peak concentration, at about the time that the concentration of plasma Listeria is 95% said peak concentration, at about the time that the concentration of plasma Listeria is 90% said peak concentration, at about the time that the concentration of plasma Listeria is 85% said peak concentration, at about the time that the concentration of plasma Listeria is 80% said peak concentration, at about the time that the concentration of plasma Listeria is 75% said peak concentration, at about the time that the concentration of plasma Listeria is 70% said peak concentration, at about the time that the concentration of plasma Listeria is 65% said peak concentration, at about the time that the concentration of plasma Listeria is 60% said peak concentration, at about the time that the concentration of plasma Listeria is 55% said peak concentration, at about the time that the concentration of plasma Listeria is 50% said peak concentration, at about the time that the concentration of plasma Listeria is 45% said peak concentration, at about the time that the concentration of plasma Listeria is 40% said peak concentration, at about the time that the concentration of plasma Listeria is 35% said peak concentration, at about the time that the concentration of plasma Listeria is 30% said peak concentration, at about the time that the concentration of plasma Listeria is 25% said peak concentration, at about the time that the concentration of plasma Listeria is 20% said peak concentration, at about the time that the concentration of plasma Listeria is 15% said peak concentration, at about the time that the concentration of plasma Listeria is 10% said peak concentration, at about the time that the concentration of plasma Listeria is 5% said peak concentration, at about the time that the concentration of plasma Listeria is 2.0% said peak concentration, at about the time that the concentration of plasma Listeria is 0.5% said peak concentration, at about the time that the concentration of plasma Listeria is 0.2% said peak concentration, or at about the time that the concentration of plasma Listeria is 0.1%, or less than, said peak concentration.

In another aspect, administration of the second reagent can begin at t=0 hours, where the administration results in a peak (or maximal plateau) in plasma concentration of the second reagent and where administration of the Listeria is initiated at about the time that the concentration of plasma level of the second reagent reaches said peak concentration, at about the time that the concentration of plasma second reagent is 95% said peak concentration, at about the time that the concentration of plasma second reagent is 90% said peak concentration, at about the time that the concentration of plasma second reagent is 85% said peak concentration, at about the time that the concentration of plasma second reagent is 80% said peak concentration, at about the time that the concentration of plasma second reagent is 75% said peak concentration, at about the time that the concentration of plasma second reagent is 70% said peak concentration, at about the time that the concentration of plasma second reagent is 65% said peak concentration, at about the time that the concentration of plasma second reagent is 60% said peak concentration, at about the time that the concentration of plasma second reagent is 55% said peak concentration, at about the time that the concentration of plasma second reagent is 50% said peak concentration, at about the time that the concentration of plasma second reagent is 45% said peak concentration, at about the time that the concentration of plasma second reagent is 40% said peak concentration, at about the time that the concentration of plasma second reagent is 35% said peak concentration, at about the time that the concentration of plasma second reagent is 30% said peak concentration, at about the time that the concentration of plasma second reagent is 25% said peak concentration, at about the time that the concentration of plasma second reagent is 20% said peak concentration, at about the time that the concentration of plasma second reagent is 15% said peak concentration, at about the time that the concentration of plasma second reagent is 10% said peak concentration, at about the time that the concentration of plasma second reagent is 5% said peak concentration, at about the time that the concentration of plasma reagent is 2.0% said peak concentration, at about the time that the concentration of plasma second reagent is 0.5% said peak concentration, at about the time that the concentration of plasma second reagent is 0.2% said peak concentration, or at about the time that the concentration of plasma second reagent is 0.1%, or less than, said peak concentration. As it is recognized that alteration of the Listeria or second reagent may occur in vivo, the above concentrations can be assessed after measurement of intact reagent, or after measurement of an identifiable degradation product of the intact reagent.

Formulations of therapeutic and diagnostic agents may be prepared for storage by mixing with physiologically acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

In some aspects, the invention also provides a kit comprising a Listeria cell, a listerial cell culture, or a lyophilized cell preparation, and a compartment. In addition, the present invention provides a kit comprising a Listeria cell, listerial cell culture, or a lyophilized cell preparation and a reagent. Also provided is a kit comprising a Listeria cell, a listerial cell culture, or a lyophilized cell preparation and instructions for use or disposal. Moreover, the present invention provides a kit comprising a Listeria cell, a listerial cell culture, or lyophilized cell preparation, and compartment and a reagent.

The present invention, in certain aspects, provides kits and methods for assessing inflammation of a tissue or organ in response to an administered attenuated Listeria. Inflammation encompasses an increase in the number (found within a biological compartment) of immune cells, leukocytes, lymphocytes, neutrophils, NK cells, CD4⁺ T cells, CD8⁺ T cells, B cells, pre-dendritic cells, dendritic cells, monocytes, macrophages, eosinophils, basophils, and/or mast cells, or any combination of the above, and the like. The kits of the present invention also provide for assessing the maturation state or activation state of one or more of the above cells. For identifying the cells and their number, an organ, tissue, or tumor can be pressed through a mesh filter to disperse the immune cells, purified using Percoll®, and identified by Fluorescence Activated Cell Sorting (FACS) (see, e.g., Woo, et al. (1994) Transplantation 58:484-491; Goossens, et al. (1990) J. Immunol. Methods 132:137-144). Inflammation can be measured as number of cells per gram tissue, or an increase in cells per gram tissue as compared with numbers from a non-inflammed state. Also available are methods for assessing Listeria-induced tissue damage, e.g., assays for leukocytosis, lymphopenia, and/or serum transaminases (Angelakopoulos, et al. (2002) Infection Immunity 70:3592-3601; Rochling (2001) Clin. Cornerstone 3:1-12; Roe (1993) Clin. Intensive Care 4:174-182).

The compositions of the invention include bulk drug compositions useful in the manufacture of non-pharmaceutical compositions (e.g., impure or non-sterile compositions) and pharmaceutical compositions (i.e., compositions that are suitable for administration to a subject or patient) which can be used in the preparation of unit dosage forms.

Moreover, the invention embraces methods for assessing efficacy of the reagents and methods of the present invention, using diagnostic tools such as ultrasound, computed tomography, magnetic resonance, analysis of point mutations, deletions, or altered DNA methylations in an oncogene, cellular proliferation markers, angiogenesis related markers, histological analysis of ploidy, or assessment of the differentiation state of the neoplastic lesion (see, e.g., Paulson (2001) Semin. Liver Dis. 21:225-236; Feitelson, et al. (2002) Oncogene 21:2593-2604; Qin and Tang (2002) World J. Gastroenterol. 8:385-392; Braga, et al. (2003) Magn. Reson. Imaging 21:871-877).

It can be determined if certain Listeria, or a composition thereof, are useful for the treatment of a particular condition or for inducing an immune response against a particular type of cancer cell, tumor or infectious agent in a mammal by testing the ability of the Listeria to stimulate an immune response in a suitable model system. The immune response can be assessed, for example, by the measurement of cytokines following administration of the Listeria to mice or other model system or by the measurement of level of certain populations of cells (e.g., NK cells) within the animal or within the liver of the animal, as demonstrated in the Examples below and in Yoshimura et al., Cancer Res, 66:1096-1104 (2006), incorporated by reference herein in its entirety. In addition, therapeutic efficacy of the vaccine composition can be assessed more directly by administration of the immunogenic composition or vaccine to the animal model such as a mouse model, followed by an assessment of survival, tumor growth, numbers of tumors, or titer of an infectious agent either in the days, weeks, or months following administration of the Listeria (e.g., for assessing innate immunity) or also after a subsequent rechallenge (e.g., for assessing acquired immunity). The hemispleen injection technique described in Jain et al., Ann. Surg. Oncol. 10:810-820 (2003), incorporated by reference herein in its entirety, and in Yoshimura et al., Cancer Res, 66:1096-1104 (2006) is particularly useful in generating a model system for investigation of the effect of the Listeria on hepatic metastases.

VIII. Uses.

The present invention provides, without limitation, methods to administer an attenuated Listeria for use in the recruitment and/or activation of immune cells for treating a proliferative condition or disorder. Methods are provided for treating a condition or disorder in a tissue or organ where the Listeria naturally accumulates, e.g., the liver. Without limiting the invention to treating liver disorders, it should be noted that L. monocytogenes is a hepatotropic bacterium. Methods are available for administration of Listeria, e.g., intravenously, subcutaneously, intramuscularly, intraperitoneally, orally, by way of the urinary tract, by way of a genital tract, by way of the gastrointestinal tract, or by inhalation (Dustoor, et al. (1977) Infection Immunity 15:916-924; Gregory and Wing (2002) J. Leukoc. Biol. 72:239-248; Hof, et al. (1997) Clin. Microbiol. Revs. 10:345-357; Schluter, et al. (1999) Immunobiol. 201:188-195; Hof (2004) Expert Opin. Pharmacother. 5:1727-1735; Heymer, et al. (1988) Infection 16(Suppl. 2):S106-S111; Yin, et al. (2003) Environ. Health Perspectives 111:524-530).

In some embodiments, the term “treatment,” as used with respect to a disease or other condition, encompasses an approach for obtaining beneficial or desired clinical results. In some embodiments, beneficial or desired clinical results include, but are not limited to, alleviation of one or more symptoms associated with a condition, prolonging survival (as compared to expected survival if not receiving treatment), stabilization (i.e., not worsening) of state of a condition, delay or slowing of progression of a condition, amelioration or palliation of the condition, remission (whether partial or total), improving a condition, curing a condition; lessening severity of a condition, and/or increasing the quality of life of one suffering from a condition. In those embodiments where the compositions described herein are used for treatment of cancer, the beneficial or desired results can include, but are not limited to, one or more of the following: reducing the proliferation of (or destroying) neoplastic or cancerous cells, inhibiting metastasis of neoplastic cells, shrinking the size of a tumor, inhibiting the growth of a tumor, regression of a tumor, remission of a cancer, decreasing symptoms resulting from the cancer, increasing the quality of life of those suffering from cancer, decreasing the dose of other medications required to treat the cancer, delaying the progression of cancer, and/or prolonging survival of patients having cancer. In some embodiments, treating a condition (e.g., a cancerous or infectious condition) comprises inhibiting or reducing the condition. In certain embodiments, treating a condition (e.g., a cancerous or infectious condition) comprises enhancing survival.

The present invention, which encompasses administering a Listeria that does not comprise a nucleic acid encoding a tumor antigen or a cancer antigen, finds use in treating tumors, cancers, and pre-cancerous disorders of the liver, gall bladder, skin, lung, muscle, heart, connective tissues, blood vessels, pancreas, mouth, tongue, throat, stomach, small intestines, large intestines, colon, rectum, prostate gland, adrenal gland, brain, nervous system, eye, spleen, bone, bone marrow, endocrine system, reticuloendothelial system, immune system, lymphatics, reproductive tract, ovary, uterus, and the like. The present invention, which encompasses administering a Listeria that does not comprise a nucleic acid encoding an antigen of an infectious organism (e.g., virus, bacterium, parasite), finds use in treating hepatitis B virus, hepatitis C virus, polyomavirus, including SV40, human papillomavirus, and the like.

The present invention, which embraces administering a Listeria that does not comprise a nucleic acid encoding a tumor antigen or a cancer antigen, finds use in preventing tumors, cancers, and pre-cancerous disorders of the liver, gall bladder, skin, lung, muscle, heart, connective tissues, blood vessels, pancreas, mouth, tongue, throat, stomach, small intestines, large intestines, colon, rectum, prostate gland, adrenal gland, brain, nervous system, eye, spleen, bone, bone marrow, endocrine system, reticuloendothelial system, immune system, lymphatics, reproductive tract, overy, uterus, and the like. The present invention, which contemplates administering a Listeria that does not comprise a nucleic acid encoding an antigen of an infectious organism (e.g., virus, bacterium, parasite), finds use in preventing infections by hepatitis B virus, hepatitis C virus, polyomavirus, including SV40, human papillomavirus, and the like.

The present invention, which encompasses administering a Listeria that does not comprise a nucleic acid encoding a tumor antigen or a cancer antigen, finds use in improving survival, i.e., survival time (in terms of days, months, and/or years), to tumors, cancers, and pre-cancerous disorders of the liver, gall bladder, skin, lung, muscle, heart, connective tissues, blood vessels, pancreas, mouth, tongue, throat, stomach, small intestines, large intestines, colon, rectum, prostate gland, adrenal gland, brain, nervous system, eye, spleen, bone, bone marrow, endocrine system, reticuloendothelial system, immune system, lymphatics, reproductive tract, ovary, uterus, and the like. The present invention, which encompasses administering a Listeria that does not comprise a nucleic acid encoding an antigen of an infectious organism (e.g., virus, bacterium, parasite), finds use in treating hepatitis B virus, hepatitis C virus, polyomavirus, including SV40, human papillomavirus, and the like.

The present invention results in the reduction of the number of abnormally proliferating cells, reduction in the number of cancer cells, reduction in the number of tumor cells, reduction in the tumor volume, reduction of the number of infectious organisms or pathogens per unit of biological fluid or tissue (e.g., serum), reduction in viral titer (e.g., serum), where it is normally reduced by at least 5%, more normally reduced by at least 10%, most normally reduced by at least 15%, preferably reduced by at least 20%, more preferably reduced by at least 25%, most normally reduced by at least 30%, usually reduced by at least 40%, more usually reduced by at least 50%, most usually reduced by at least 60%, conventionally reduced by at least 70%, more conventionally reduced by at least 80%, most conventionally reduced by at least 90%, and still most conventionally reduced by at least 99%. The unit of reduction can be, without limitation, number of tumor cells/mammalian subject; number of tumor cells/liver; number of tumor cells/spleen; mass of tumor cells/mammalian subject; mass of tumor cells/liver; mass of tumor cells/spleen; number of viral particles or viruses or titer per gram of liver; number of viral particles or viruses or titer per cell; number of viral particles or viruses or titer per ml of blood; and the like.

The invention provides methods of treating a mammal which has a cancerous condition or which comprises a tumor, cell, or infectious agent. In some embodiments, the cancer or tumor is metastatic. In some embodiments, the cancerous condition is a cancer or tumor of the liver. In some embodiments, the condition comprises a cancer that has metastasized to the liver. In some embodiments, the cancer cells or tumors of the liver are metastatic cells from the gastrointestinal tract, hepatocellular carcinoma cells, angiosarcoma cells, or epithelioid hemangioendothelioma cells. In some embodiments, the cancer is colon cancer.

The present invention provides reagents and methods for stimulating innate response as mediated by, e.g., NK cells, NKT cells, dendritic cells and other APCs, CD4⁺ T cells, CD8⁺ T cells, and gammadelta T cells.

Provided are reagents and methods for stimulating innate response mediated by, e.g., an APC, an APC that migrates to the liver, an APC that is generated to mature in the liver, or an APC that is located in the liver, such as a dendritic cell (DC), Kupfer cell, or liver sinusoidal endothelial cell (LSEC). The present invention is not limited, unless specified explicitly or by context, to the receptors, signaling molecules, or cells that mediate the innate response.

The growth medium used to prepare a Listeria can be characterized by chemical analysis, high pressure liquid chromatography (HPLC), mass spectroscopy, gas chromatography, spectroscopic methods, and the like. The growth medium can also be characterized by way of antibodies specific for components of that medium, where the component occurs as a contaminant with the Listeria, e.g., a contaminant in the listerial powder, frozen preparation, or cell paste. Antibodies, specific for peptide or protein antigens, or glycolipid, glycopeptide, or lipopeptide antigens, can be used in ELISA assays formulated for detecting animal-origin contaminants. Antibodies for use in detecting antigens, or antigenic fragments, of animal origin are available (see, e.g., Fukuta, et al. (1977) Jpn. Heart J. 18:696-704; DeVay and Adler (1976) Ann. Rev. Microbiol. 30:147-168; Cunningham, et al. (1984) Infection Immunity 46:34-41; Kawakita, et al. (1979) Jpn. Cir. J. 43:452-457; Hanly, et al. (1994) Lupus 3:193-199; Huppi, et al. (1987) Neurochem. Res. 12:659-665; Quackenbush, et al. (1985) Biochem. J. 225:291-299). The invention supplies kits and diagnostic methods that facilitate testing the Listeria's influence on the immune system. Testing can involve comparing one strain of Listeria with another strain of Listeria, or a parent Listeria strain with a mutated Listeria strain. Methods of testing comprise, e.g., phagocytosis, spreading, antigen presentation, T cell stimulation, cytokine response, host toxicity, LD₅₀, and efficacy in ameliorating a pathological condition.

The present invention provides methods to increase survival of a subject, host, patient, test subject, experimental subject, veterinary subject, and the like, to a proliferative disorder, a cancer, a tumor, an immune disorder, and/or an infectious agent. The infectious agent can be a virus, bacterium, or parasite, or any combination thereof. The method comprises administering an attenuated Listeria, for example, as a suspension, bolus, gel, matrix, injection, or infusion, and the like. The administered Listeria increases survival, as compared to an appropriate control (e.g., nothing administered or an administered placebo, and the like) by usually at least one day; more usually at least four days; most usually at least eight days, normally at least 12 days; more normally at least 16 days; most normally at least 20 days, often at least 24 days; more often at least 28 days; most often at least 32 days, conventionally at least 40 days, more conventionally at least 48 days; most conventionally at least 56 days; typically by at least 64 days; more typically by at least 72 days; most typically at least 80 days; generally at least six months; more generally at least eight months; most generally at least ten months; commonly at least 12 months; more commonly at least 16 months; and most commonly at least 20 months, or more.

The invention provides each of the above-disclosed embodiments, where the administered attenuated Listeria are administered as a composition that is at least 90% free of other types of bacteria, that is at least 95% free of other types of bacteria, that is at least 99% free of other types of bacteria, or that is at least 99.9% free of other types of bacteria. Other types of bacteria include, e.g., a serotype of L. monocytogenes other than that disclosed above. Other types of bacteria also include, e.g., L. welshimeri, L. seeligeri, L. innocua, L. grayi, S. typhimurium (Silva, et al. (2003) Int. J. Food Microbiol. 81:241-248; Pini and Gilbert (1988) Int. J. Food Microbiol. 6:317-326; Council of Experts (2003) Microbiological Tests in The United States Pharmacopeia, The National Formulary, Board of Trustees, pp. 2148-2162).

Yet another embodiment of the present invention provides a method of preventing a proliferative disorder in a subject, or mammalian subject, at risk for the disorder, comprising administering an effective number or amount of a killed but metabolically active Listeria. Provided is the above method, where the killed but metabolically active Listeria comprises one or more of: (a) a cross-link of the listerial genome; (b) a cross-link of the listerial genome comprising a nucleic acid targeting compound; (c) a cross-link of the listerial genome comprising a psoralen; (d) an interstrand cross-link of the listerial genome comprising a nucleic acid targeting compound; and/or an interstrand cross-link of the listerial genome comprising a nucleic acid targeting compound; (e) an attenuation in a virulence factor; (f) an attenuation in actA, such as ΔactA; (g) an attenuation in inlB, such as ΔinlB; (h) an attenuation in actA and inlB; (i) an attenuated uvrA, uvrB, uvrC, or uvrAB, such as ΔuvrAB; (j) an attenuated uvrAB, an interstrand psoralen cross-link, and an attenuated actA; (k) an attenuated uvrAB, an interstrand psoralen cross-link, and an attenuated inlB; (l) ΔuvrAB, an interstrand psoralen cross-link, and ΔactAΔinlB.

The invention provides a Listeria bacterium, or a Listeria strain, that is killed but metabolically active (KBMA) (see, e.g., Brockstedt, et al. (2005) Nat. Med. [July 24 epub ahead of print]). A KBMA Listeria bacterium is metabolically active, but cannot form a colony, e.g., on agar. An inactivating mutation in at least one DNA repair gene, e.g., ΔuvrAB, enables killing of Listeria using concentrations of a nucleic acid cross-linking agent (e.g., psoralen) at low concentrations, where these concentrations are sufficient to prevent colony formation but not sufficient to substantially impair metabolism. The result of limited treatment with psoralen/UVA light, and/or of treatment with a nucleic acid cross-linking agent that is highly specific for making interstrand genomic cross links, is that the bacterial cells are killed but remain metabolically active.

Each of the above disclosed methods contemplates administering a composition comprising a Listeria and an excipient, a Listeria and a carrier, a Listeria and buffer, a Listeria and a reagent, a Listeria and a pharmaceutically acceptable carrier, a Listeria and an agriculturally acceptable carrier, a Listeria and a veterinarily acceptable carrier, a Listeria and a stabilizer, a Listeria and a preservative, and the like.

The present invention provides, in some aspects, reagents and methods for treating conditions that are both cancerous (neoplasms, malignancies, cancers, tumors, and/or precancerous disorders, dysplasias, and the like) and infectious (infections). Provided are reagents and methods for treating disorders that are both cancerous (neoplasms, malignancies, cancers, tumors, and/or precancerous disorders, dysplasias, and the like) and infectious. With infection with certain viruses, such as papillomavirus and polyoma virus, the result can be a cancerous condition, and here the condition is both cancerous and infectious. A condition that is both cancerous and infectious can be detected, as a non-limiting example, where a viral infection results in a cancerous cell, and where the cancerous cell expresses a viral-encoded antigen. As another non-limiting example, a condition that is both cancerous and infectious is one where immune response against a tumor cell involves specific recognition against a viral-encoded antigen (See, e.g., Montesano, et al. (1990) Cell 62:435-445; Ichaso and Dilworth (2001) Oncogene 20:7908-7916; Wilson, et al. (1999) J. Immunol. 162:3933-3941; Daemen, et al. (2004) Antivir. Ther. 9:733-742; Boudewijn, et al. (2004) J. Natl. Cancer Inst. 96:998-1006; Liu, et al. (2004) Proc. Natl. Acad. Sci. USA 101:14567-14571).

In some embodiments, the methods described herein are applied to a primate. In some embodiments, the methods described herein are applied to a dog, cat, mouse, rat, monkey, rabbit, or horse. In some embodiments, the methods described herein are applied to humans.

The broad scope of this invention is best understood with reference to the following examples, which are not intended to limit the invention to any specific embodiments.

### EXAMPLES

I. General Methods.

Standard methods of biochemistry and molecular biology are described (see, e.g., Maniatis, et al. (1982) Molecular Cloning, A Laboratory Manual, Cold Spring Harbor Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.; Innis, et al. (eds.) (1990) PCR Protocols: A Guide to Methods and Applications, Academic Press, N.Y. Standard methods are also found in Ausbel, et al. (2001) Curr. Protocols in Mol. Biol., Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). Methods for producing fusion proteins are described (see, e.g., Invitrogen (2005) Catalogue, Carlsbad, Calif.; Amersham Pharmacia Biotech. (2005) Catalogue, Piscataway, N.J.; Liu, et al. (2001) Curr. Protein Pept. Sci. 2:107-121; Graddis, et al. (2002) Curr. Pharm. Biotechnol. 3:285-297). Splice overlap extension PCR, and related methods, are described (see, e.g., Horton, et al. (1990) Biotechniques 8:528-535; Horton, et al. (1989) Gene 77:61-68; Horton (1995) Mol Biotechnol. 3:93-99; Warrens, et al. (1997) Gene 186:29-35; Guo and Bi (2002) Methods Mol. Biol. 192:111-119; Johnson (2000) J. Microbiol. Methods 41:201-209; Lantz, et al. (2000) Biotechnol. Annu. Rev. 5:87-130; Gustin and Burk (2000) Methods Mol. Biol. 130:85-90; QuikChange® Mutagenesis Kit, Stratagene, La Jolla, Calif.). Engineering codon preferences of signal peptides, secretory proteins, and heterologous antigens, to fit the optimal codons of a host are described (Sharp, et al. (1987) Nucl. Acids Res. 15:1281-1295; Uchijima, et al. (1998) J. Immunol. 161:5594-5599).

Methods for protein purification such as immunoprecipitation, column chromatography, electrophoresis, isoelectric focusing, centrifugation, and crystallization, are described (Coligan, et al. (2000) Current Protocols in Protein Science, Vol. 1, John Wiley and Sons, Inc., New York). Chemical analysis, chemical modification, post-translational modification, and glycosylation of proteins is described. See, e.g., Coligan, et al. (2000) Current Protocols in Protein Science, Vol. 2, John Wiley and Sons, Inc., New York; Walker (ed.) (2002) Protein Protocols Handbook, Humana Press, Towota, N.J.; Lundblad (1995) Techniques in Protein Modification, CRC Press, Boca Raton, Fla. Techniques for characterizing binding interactions are described (Coligan, et al. (2001) Current Protocols in Immunology, Vol. 4, John Wiley and Sons, Inc., New York; Parker, et al. (2000) J. Biomol. Screen. 5: 77-88; Karlsson, et al. (1991) J. Immunol. Methods 145:229-240; Neri, et al. (1997) Nat. Biotechnol. 15:1271-1275; Jonsson, et al. (1991) Biotechniques 11:620-627; Friguet, et al. (1985) J. Immunol. Methods 77: 305-319; Hubble (1997) Immunol. Today 18:305-306; Shen, et al. (2001) J. Biol. Chem. 276:47311-47319).

Software packages for determining, e.g., antigenic fragments, leader sequences, protein folding, functional domains, glycosylation sites, and sequence alignments, are available (see, e.g., Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690). Methods for determining coding sequences (CDS) are available (Furono, et al. (2003) Genome Res. 13:1478-1487).

Computer algorithms (e.g., BIMAS; SYFPEITHI) for identifying peptides that bind to MHC Class I and/or MHC Class II are available (Thomas, et al. (2004) J. Exp. Med. 200:297-306). These algorithms can provide nucleic acids of the present invention that encode proteins comprising the identified peptides.

Sequences of listerial proteins and nucleic acids can be found on the world wide web at: (1) ncbi.nlm.nih.gov; (2) genolist.Pasteur (with clicking on “listilist”); and (3) tigr.org (with clicking on “comprehensive microbial resource”).

Methods are available for assessing internalization of a Listeria by an APC, and for assessing presentation of listerial-encoded antigens by the APC. Methods are also available for presentation of these antigens to T cell, and for assessing antigen-dependent priming of the T cell. A suitable APC is murine DC 2.4 cell line, while suitable T cell is the B3Z T cell hybridoma (see, e.g., U.S. Provisional Pat. Appl. Ser. No. 60/490,089 filed Jul. 24, 2003; Shen, et al. (1997) J. Immunol. 158:2723-2730; Kawamura, et al. (2002 J. Immunol. 168:5709-5715; Geginat, et al. (2001) J. Immunol. 166:1877-1884; Skoberne, et al. (2001) J. Immunol. 167:2209-2218; Wang, et al. (1998) J. Immunol. 160:1091-1097; Bullock, et al. (2000) J. Immunol. 164:2354-2361; Lippolis, et al. (2002) J. Immunol. 169:5089-5097). Methods for preparing dendritic cells (DCs), ex vivo modification of the DCs, and administration of the modified DCs, e.g., for the treatment of a cancer, pathogen, or infective agent, are available (see, e.g., Ribas, et al. (2004) J. Immunother. 27:354-367; Gilboa and Vieweg (2004) Immunol. Rev. 199:251-263; Dees, et al. (2004) Cancer Immunol. Immunother. 53:777-785; Eriksson, et al. (2004) Eur. J. Immunol. 34:1272-1281; Goldszmid, et al. (2003) J. Immunol. 171:5940-5947; Coughlin and Vonderheide (2003) Cancer Biol. Ther. 2:466-470; Colino and Snapper (2003) Microbes Infect. 5:311-319).

Elispot assays and intracellular cytokine staining (ICS) for characterizing immune cells are available (see, e.g., Lalvani, et al. (1997) J. Exp. Med. 186:859-865; Waldrop, et al. (1997) J. Clin. Invest. 99:1739-1750; Hudgens, et al. (2004) J. Immunol. Methods 288:19-34; Goulder, et al. (2001) J. Virol. 75:1339-1347; Goulder, et al. (2000) J. Exp. Med. 192:1819-1831; Anthony and Lehman (2003) Methods 29:260-269; Badovinac and Harty (2000) J. Immunol. Methods 238:107-117).

Methods for using animals in the study of cancer, metastasis, and angiogenesis, and for using animal tumor data for extrapolating human treatments are available (see, e.g., Hirst and Balmain (2004) Eur J Cancer 40:1974-1980; Griswold, et al. (1991) Cancer Metastasis Rev. 10:255-261; Hoffman (1999) Invest. New Drugs 17:343-359; Boone, et al. (1990) Cancer Res. 50:2-9; Moulder, et al. (1988) Int. J. Radiat. Oncol. Biol. Phys. 14:913-927; Tuveson and Jacks (2002) Curr. Opin. Genet. Dev. 12:105-110; Jackson-Grusby (2002) Oncogene 21:5504-5514; Teicher, B. A. (2001) Tumor Models in Cancer Research, Humana Press, Totowa, N.J.; Hasan, et al. (2004) Angiogenesis 7:1-16; Radovanovic, et al. (2004) Cancer Treat. Res. 117:97-114; Khanna and Hunter (2004) Carcinogenesis September 9 [epub ahead of print]; Crnic and Christofori (2004) Int. J. Dev. Biol. 48:573-581).

Colorectal cancer hepatic metastases can be generated using primary hepatic injection, portal vein injection, or whole spleen injection of tumor cells (see, e.g., Suh, et al. (1999) J. Surgical Oncology 72:218-224; Dent and Finley-Jones (1985) Br. J. Cancer 51:533-541; Young, et al. (1986) J. Natl. Cancer Inst. 76:745-750; Watson, et al. (1991) J. Leukoc. Biol. 49:126-138).

II. Methods Relating to Animal Tumor Models.

The Listeria monocytogenes strains used in the present work are described (see, e.g., Brockstedt, et al. (2004) Proc. Natl. Acad. Sci. USA 101:13832-13837). L. monocytogenes ΔactAΔinlB is available from American Type Culture Collection (ATCC) at PTA-5562. L. monocytogenes ΔactAΔuvrAB is available from ATCC at PTA-5563. Other listerial strains are available (see, e.g., U.S. Pat. Applic. 2004/0013690 of Portnoy, et al.).

A number of animal tumor models were used, where these models utilized BALB/c mice and the syngeneic colorectal cancer line CT26 (ATCC CRL-2638). The models used in the present invention included: (1) Subcutaneous CT26 tumors; and (2) Injection of tumor cells into half of a surgically bisected spleen, followed by immediate excision of the injected half (hemispleen model). The hemispleen model established colorectal cancer hepatic metastases without producing a primary tumor in the spleen. The hemispleen method allows seeding of the liver with tumor cells through the portal circulation without the presence of a primary tumor in the injected spleen. Where indicated, mice were treated with GM-CSF secreting tumor vaccines, where vaccination was initiated three days after tumor challenge.

CT26, an immortal mouse colorectal cancer cell line (generated by exposure of BALB/c background mice rectal tissue to methylcholanthrine) was used to establish tumors used in the present study (Corbett, et al. (1975) Cancer Res. 35:2434-2439). The vaccine cell line was derived from CT26 cells transduced to secrete murine GM-CSF using a replication defective MFG retroviral vector (Dranoff, et al. (1993) Proc. Natl. Acad. Sci. USA 90:3539-3543). Tumor cell lines were grown in tumor media containing (vol/vol) 900 ml RPMI media, 100 ml 10% heat inactivated fetal calf serum, 10 ml penicillin/streptomycin (10,000 U/ml), 10 ml MEM non-essential amino acids (10 mM), 10 ml HEPES buffer (1 M), 10 ml sodium pyruvate (100 mM), and 10 ml L-glutamate (200 mM).

For subcutaneous tumor model studies, BALB/c mice were injected with 0.1 million CT26 colorectal cancer cells suspended in 0.05 ml HBSS below the left lower nipple. Tumors were allowed to grow for 28 days in control mice. Tumors were measured bi-weekly in three dimensions using calipers. Treated mice were vaccinated with GM-CSF secreting tumor cells on a bi-weekly basis.

Hemispleen injections were as follows. BALB/c mice were anaesthetized and the spleen exposed. The spleen was divided into two hemispleens, leaving the vascular pedicles intact. Using a 27 gauge needle, about 0.1 million viable CT26 cells in 0.4 ml HBSS buffer were injected into the spleen, thus allowing cells to flow to the liver. The vascular pedicle draining the cancer-contaminated hemispleen was ligated with a clip, and the CT26-contaminated hemispleen was excised, leaving a functional hemispleen free of tumor cells.

In all studies, except for one study as indicated, the vaccine (tumor cell vaccine) was prepared by treating the tumor cells with gamma-rays. In this one study, the vaccine was prepared by photochemical treatment (psoralen and UV light). In all studies, except where indicated, the number of pathologic CT26 tumor cells used in the innoculum (not the attenuated CT26 cells used in the vaccine) administered was about 0.1 million cells. Subjecting tumor cells with gamma-rays or photochemical treatment results in attenuated tumor cells that can provide an antigen or antigens, and can express an immunomodulating agent such as GM-CSF, but cannot grow and/or replicate. Where a nucleic acid encoding GM-CSF is used as part of a vaccine, the terms “GM vaccine” and “GM-CSF vaccine” may be used interchangeably.

In general, mice receiving Listeria weighed 20-25 grams, and had a surface area of about 0.0066 square meters.

Anti-CD16/32, anti-CD69, anti-CD25, and anti-CD3 were from eBioscience (San Diego, Calif.). Total numbers of NK cells and NK-T cells was determined using the following cocktail: CD45 to stain all leukocytes, to separate these from residual liver cells, and CD19 to eliminate B cells from the analysis. Then, the two parameter plot of CD3 versus DX-5 was used to identify T cells (CD3⁺DX-5⁻), NK cells (DX-5⁺CD3⁻), and NK-T cells (CD3⁺DX-5⁺).

III. Administration of Attenuated Listeria (with No Vaccine) Enhanced Survival to Liver Tumors (Generated Via Hemispleen Injection Model).

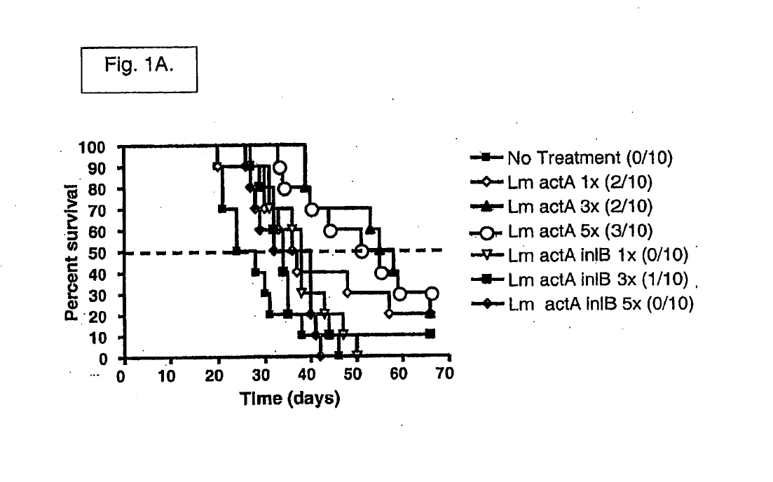

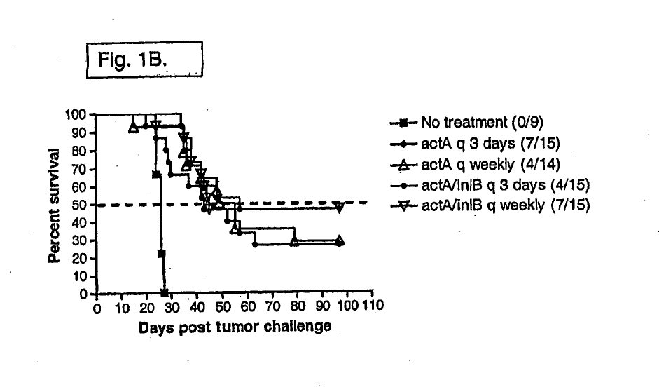

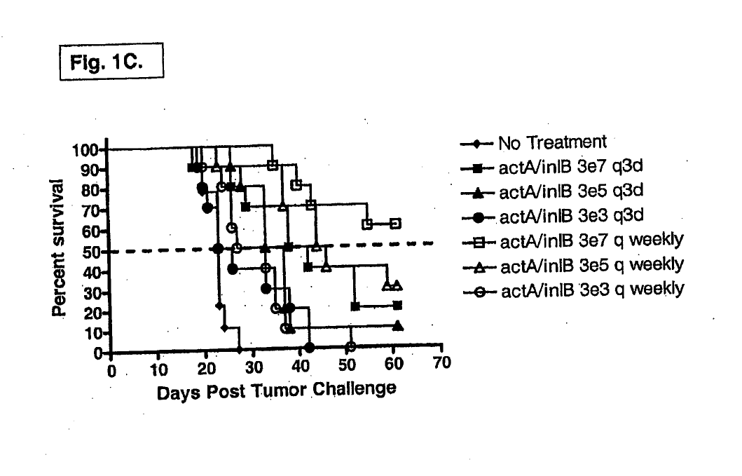

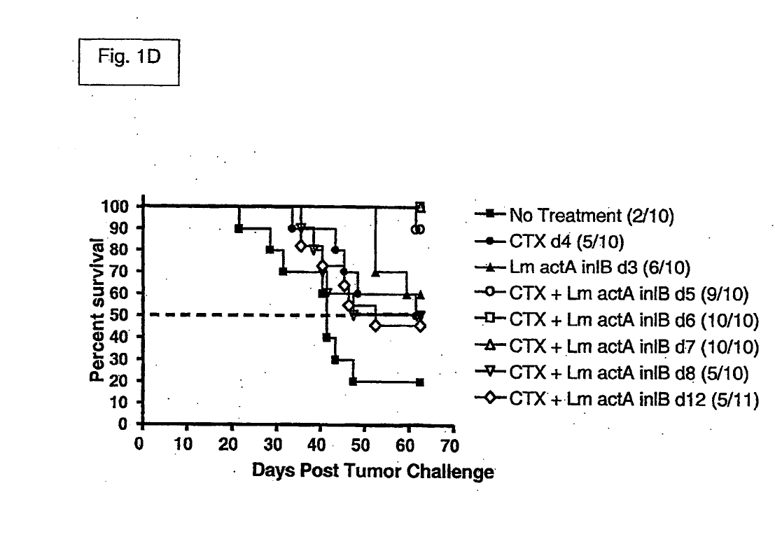

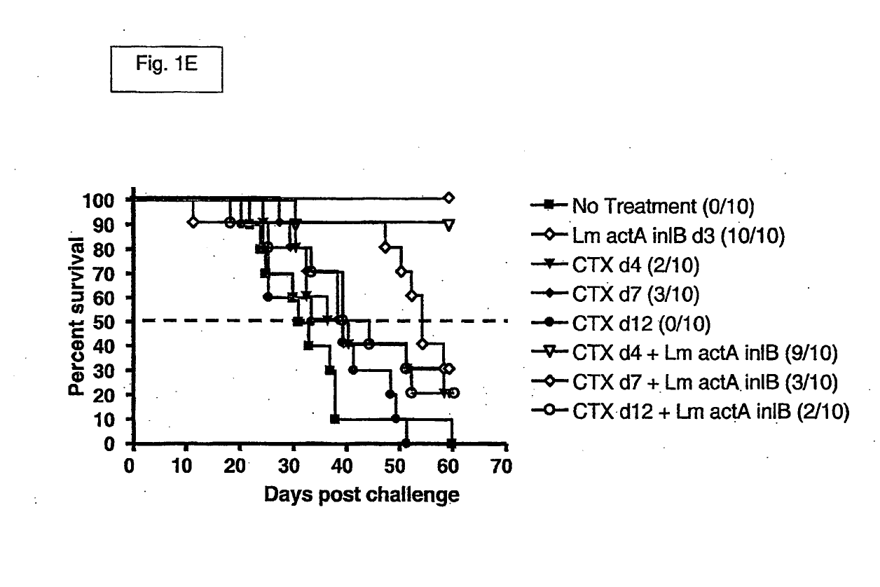

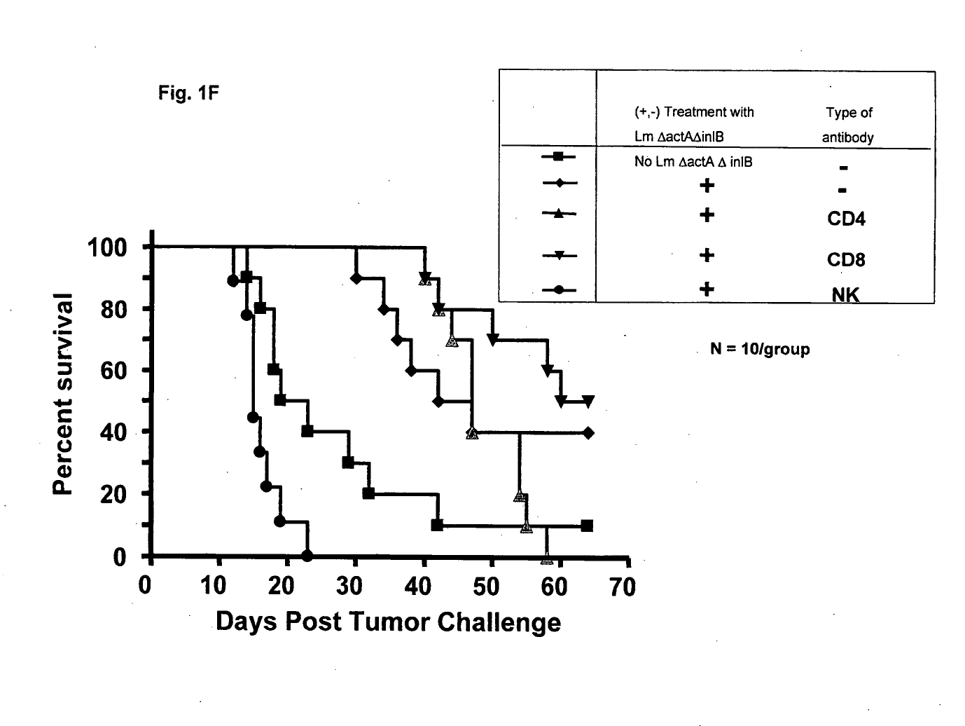

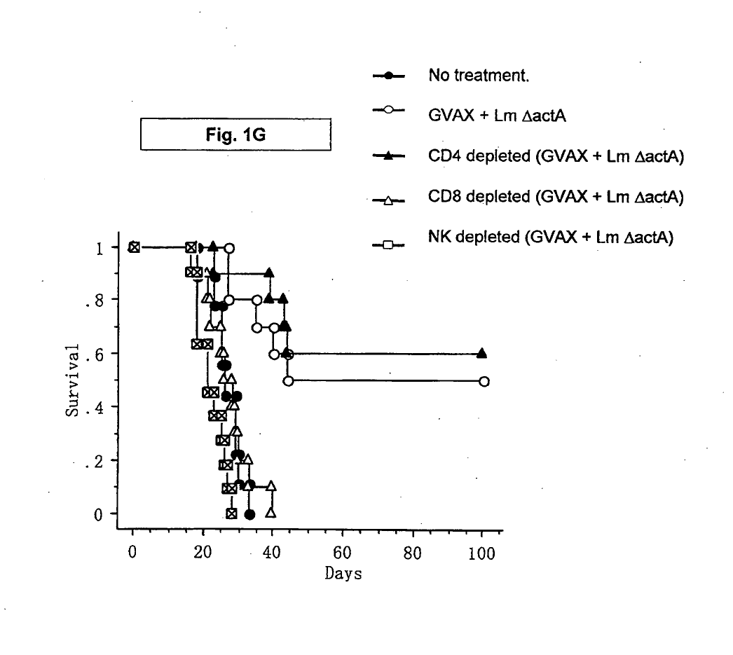

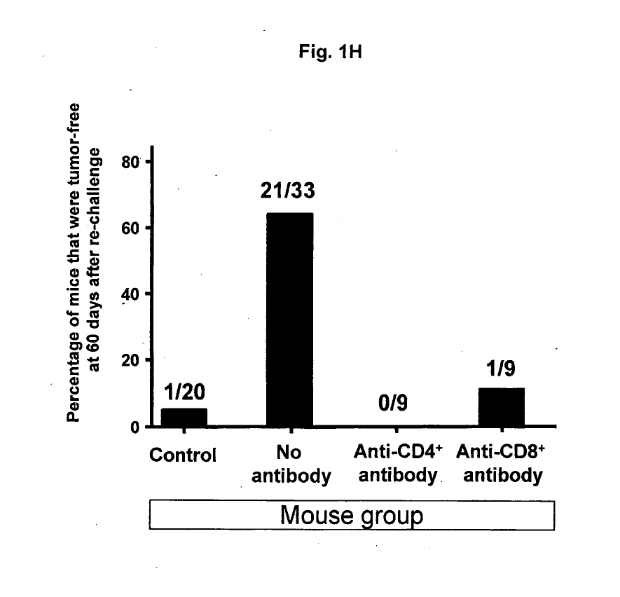

Hepatic tumors were induced in mice as follows. CT26 tumor cells were administered to all mice on day zero (t=0 days) to initiate hepatic tumor formation. Mice were treated intravenously (i.v.) with no Listeria (-▪- lower curve of small squares), with the indicated amount of Listeria ΔactA (-⋄- open diamonds; -▴- triangles; -•- filled circles); or with the indicated amount of Listeria ΔactAΔinlB (-∇- inverted triangles; -▾- upper curve of large squares; -♦- filled diamonds) (FIG. 1A).

The following concerns the number of doses of Listeria given to the mice. “1×” means that the indicated Listeria strains were administered only at t=3 days post tumor implant (only one dose). “3×” means that the indicated Listeria strains were administered at t=3 days, 6 days, and 9 days. “5×” means that the indicated Listeria strains were administered at t=3 days, 6 days, 9 days, 12 days, and 15 days. The number of administered Listeria ΔactA cells was about 1×10⁷ colony forming units (CFU) while the number of Listeria ΔactAΔinlB given was about 2×10⁷ CFU (FIG. 1A).

The results were as follows. Where tumor-bearing mice received no Listeria, 50% of the mice died by 25 days, while 100% died by day 42. In contrast, mice treated with Listeria ΔactA or Listeria ΔactAΔinlB showed increased survival. For example, at t=25 days, all mice receiving either Listeria ΔactA or Listeria ΔactAΔinlB showed a survival rate of at least 90%. The survival rate was the greatest with Listeria ΔactA, where Listeria ΔactA was provided at 3× or 5× doses (FIG. 1A).

In a separate study (FIG. 1B), CT26 tumor cell-treated mice were given no Listeria (-▪-; squares); Listeria ΔactA (every three days, three doses in all) (-♦-; diamonds); Listeria ΔactA (weekly, three doses in all) (-Δ-; open triangles); Listeria ΔactAΔinlB (every three days, three doses in all) (-•- filled circles); or Listeria ΔactAΔinlB (weekly, three doses in all) (−7-; inverted open triangles). The results demonstrated that with no treatment, all animals died before t=30 days, whereas Listeria-treatment resulted in survival of up to 50% of the animals at t=100 days (FIG. 1B). Again, the Listeria used to provide data for FIGS. 1A, B were not engineered to contain any nucleic acid encoding heterologous antigen.

In still another study (FIG. 1C), CT26 tumor cell-innoculated mice were treated as follows. Bacteria were grown on yeast broth with no glucose, where bacteria were administered i.v. Mice were given no Listeria (-♦-; diamonds); Listeria ΔactAΔinlB (3×10⁷ CFU, every three days, three doses in all) (-▪-; squares); Listeria ΔactAΔinlB (3×10⁵ CFU, every three days, three doses in all) (-▴-; filled triangles); Listeria ΔactAΔinlB (3×10³ CFU, every three days, three doses in all) (-•-; filled circles); Listeria ΔactAΔinlB (3×10⁷ CFU, weekly, three doses in all) (-□-; open squares); Listeria ΔactAΔinlB (3×10⁵ CFU, weekly, three doses in all) (-Δ-; open triangles); Listeria ΔactAΔinlB (3×10³ CFU, weekly, three doses in all) (-O-; open circles). An observation that can be made is that, with no treatment, all of the animals died by t=30 days, while mice receiving Listeria ΔactAΔinlB (3×10⁷ CFU) weekly (-□-; open squares) had the greatest survival.

Studies of tumor-bearing mice treated with Listeria, where the Listeria was not engineered to express a heterologous antigen, were continued, where these continued studies included administration of cyclophosphamide (Cytoxan®; CTX) (FIGS. 1D and 1E). The day of CTX treatment (t=day 4) was held constant, while the day of Listeria administration was varied (FIG. 1D). When administered, CTX was provided at 50 mg/kg (i.p.). All doses of L. monocytogenes were 3×10⁷, where the bacteria were prepared by growing in yeast broth with no glucose. The following provides a legend to the figure: Data from mice with no treatment (-▪-; filled squares); treated with CTX only (day 4 injection) (-•-; filled circles); Listeria ΔactAΔinlB only (Listeria administered on days 3, 10, 17) (-▴-; filled triangles); CTX (day 4) with Listeria ΔactAΔinlB (Listeria administered on days 5, 12, and 19) (-O-; open circles); CTX (day 4) with Listeria ΔactAΔinlB (Listeria administered on days 6, 13, and 20) (-□-; open squares); CTX (day 4) with Listeria ΔactAΔinlB (Listeria administered on days 7, 14, 21) (-Δ-; open triangles); CTX (day 4) with Listeria ΔactAΔinlB (Listeria administered on days 8, 15, and 22) (-∇-; open inverted triangles); and CTX (day 4) with Listeria ΔactAΔinlB (Listeria administered on days 12, 19, and 26) (-⋄-; open diamonds). The results were as follows. With no treatment (no CTX; no Listeria), survival of the mice at about t=50 days was about 20% (-▪-closed squares). With CTX only, survival was about 60% at t=50 days (-•-; filled circles). In some protocols that included both CTX and bacteria, survival was between 90-100% after t=60 days (Listeria administered at t=day 5, 6, or 7).

FIG. 1D demonstrates that administering CTX (at t=4 days) alone results in some increase in survival, and that administering CTX (at t=4 days) plus Listeria (Listeria administered at days 5, 12, and 19; Listeria administered at days 6, 13, and 20; or Listeria at days 7, 14, and 21) results in even greater survival.

The following demonstrates that CTX+Listeria can improve survival, and illustrates tests showing how long administration of this combination can be delayed and where the delated combination still improved survival.

FIG. 1E demonstrates combination therapy, and the effects of delaying combination therapy. In this figure, “combination therapy” means the combination of Listeria ΔactAΔinlB (not engineered to express any heterologous antigen) plus cyclophosphamide. Where no treatment was give, half the animals died by about t=32 days. When administered, CTX was provided at 50 mg/kg (i.p.). All doses of L. monocytogenes were 3×10⁷, where the bacteria were prepared by growing in yeast broth with no glucose.

Where the combination dose schedule was started at t=4 days (CTX at day 4 and Listeria at days 5, 12, and 19) (-∇-; open inverted triangles), near maximal survival was found, and here 90% of the animals were surviving at t=60 days. Where the combination dose schedule was delayed somewhat, and started at t=7 days (CTX at day 7 and Listeria at days 8, 15, and 22), about 90% of the animals were surviving at t=48 days, with about half surviving at t=53 days (-⋄-; open diamonds). With further delay in initiating combination therapy, and started at t=12 days (CTX at t=12 days and Listeria at days 13, 20, and 27), survival was relatively poor (-O-; open circles) (FIG. 1E).

The experiments for which results are shown in FIGS. 1F, 1G, and 1H involve the use of depleting antibodies which, when injected in a mouse, deplete a predetermined type of immune cell, for example, CD8⁺ T cells or NK cells.

The results shown in FIG. 1F provide insight into the mechanisms by which Listeria (not engineered to express any tumor antigen) improves survival to tumors in the absence of a second vaccine. (GVAX was not used in this particular experiment.)

The experimental methods for FIG. 1F were as follows: On Day 0, female Balb/c mice were implanted with 1×15 CT26 cells via hemispleen surgery, and randomized into different treatment groups. CD4⁺ and CD8⁺ T cell and NK cell depletion was initiated one week prior to tumor cell implantation followed by two additional injections on Days 6 and 13 of the GK1.5 (anti-CD4), 2.43 (anti-CD8) and anti-AsialoGM (anti-NK) antibodies, respectively. Depletion of the respective lymphocyte population was confirmed by flow cytometry in separate cohorts of mice. Three weekly treatments with 3×10⁷ cfu of Lm ΔactAΔinlB were initiated on Day 3, except for the control, and mice were followed for survival.

FIG. 1F shows the percent survival of the mice inoculated with CT26 tumors, where the CT26-tumor cell inoculated mice were treated with Lm ΔactAΔinlB or with no Lm ΔactAΔinlB, as indicated. The treated mice either received no antibody or received antibodies that specifically deplete CD4⁺ T cells; CD8⁺ T cells; or NK cells, as indicated. The results demonstrated maximal, or near maximal, survival where mice received Lm ΔactAΔinlB after receiving no depleting antibodies; Lm ΔactAΔinlB after receiving anti-CD4⁺ T cell antibodies; or Lm ΔactAΔinlB after receiving anti-CD8⁺ T cell antibodies). In contrast, low survival occurred where Lm ΔactAΔinlB was not administered, or where Lm ΔactAΔinlB was administered to mice who had received anti-NK cell antibodies. These results indicate that following the initial inoculation with tumor cells, Lm-mediated stimulation of NK cells is of major importance for survival to tumors, whereas CD8⁺ T cells and CD4⁺ T cells are relatively unimportant to survival.

The following addresses the mechanisms by which Listeria (not engineered to express any tumor antigen), in combination with GM-CSF vaccine, improves survival to tumors. FIG. 1G reveals survival of mice to CT26 tumors, where CT26-tumor cell inoculated mice were treated with Listeria ΔactA plus GM-CSF vaccine, along with an agent that specifically depletes CD4⁺ T cells (-▴-; GK1.5 antibody), CD8⁺ T cells (-Δ-; 2.43 antibody), or NK cells (- [symbol] -; anti-asialo-GM1 antibody), or no other agent (-•-; no treatment, NT). Treatment with the indicated antibodies was for two weeks prior to implantation of intra-hepatic tumor cells. Antibody-dependent depletion of over 90% of CD4⁺ T cells, CD8⁺ T cells, or NK cells, was confirmed by flow cytometry analysis of liver and spleen from one or two animals from each group. The results demonstrate that maximal survival of tumor cell-bearing mice occurred where mice were treated with Listeria plus vaccine (-O-) or with Listeria plus vaccine along with the CD4⁺ T cell-depleting antibody (-▴-; GK 1.5 antibody). In contrast, survival was poor (as poor as with no administered therapeutic agents) where tumor cell-bearing mice were treated with Listeria plus vaccine along with an antibody that depletes CD8⁺ T cells or with an antibody that depletes NK cells.

In some embodiments, the present invention provides a method to improve survival to a cancer, by administering a Listeria plus attenuated tumor cells, where the attenuated tumor cells share antigenic properties with the cancer, and where the survival to the cancer is mediated by, and not limited to, NK cells and/or CD8⁺ T cells. Moreover, the present invention also provides, in some embodiments, a method to improve survival to an infectious agent (e.g., virus, bacteria, parasite), by administering a Listeria plus attenuated infectious agent, where the attenuated infectious agent shares antigenic properties with the infectious agent, and where the survival to the infectious agent is mediated by, and not limited to, NK cells and/or CD8⁺ T cells.

FIG. 1H shows the results of a depletion study where long term survivors that were previously injected with Lm ΔactAΔinlB following inoculation with CT26 tumor cells were re-challenged with CT26 tumor cells. Briefly, experimental mouse groups were inoculated with CT26 tumor cells (1×10⁵ CT26 cells), via the hemispleen model, at t=0 days, and were subsequently injected with Lm ΔactAΔinlB (1×10⁷ bacteria/dose) at t=3, 10, and 17 days (three doses). At t>100 days, about 50-60% of the mice were still alive, and these were the long term survivors. To evaluate tumor specific T cell immunity, long-term survivors were rechallenged subcutaneously with CT26 cells (2×10⁵ CT26 cells).

Prior to the CT26 tumor cell rechallenge, anti-CD4 antibodies or anti-CD8 antibodies were administered to some of the long-term survivors. The anti-CD4 antibody and anti-CD8 antibodies used in the experiment were prepared at Cerus Corporation, Concord, Calif., although anti-CD4 antibodies and anti-CD8 antibodies suitable for depleting experiments are commercially available (e.g., Invitrogen, Carlsbad, Calif.; R & D Systems, Minneapolis, Minn.). The depleting antibodies were injected (0.25 mg injected, i.p.) eight, four, and one day prior to the CT26 cell re-challenge. T cell subsets depletion was confirmed by flow cytometry analysis. Survival of mice to the CT26 cell re-challenge was determined after waiting at least 60 days after the CT26 cell re-challenge dose.

At the time of the re-challenge of the experimental mice, naive mice (controls) were also inoculated with CT26 cells. The control mice had never been earlier exposed to either CT26 tumor cells or Lm ΔactAΔinlB, that is, they were naive for both CT26 cells and for Lm ΔactAΔinlB.

The results, shown in FIG. 1H, demonstrate that in the control group, only one out of 20 mice survived the CT26 cell re-challenge. In the experimental group (i.e., the long-term survivors), about two thirds of the mice (21 out of 33 mice) survived the tumor cell re-challenge. However, where experimental mice had also received either anti-CD4 antibody or anti-CD8 antibody, most of the mice died in response to the tumor cell re-challenge. These results demonstrate that Lm ΔactAΔinlB, an engineered bacterium that does not contain any nucleic acid encoding a tumor antigen, can stimulate long-term tumor-specific adaptive (memory) immune response, and that this long-term adaptive immune response was both CD4⁺ T cell and CD8⁺ T cell dependent.

IV. Listeria did not Provoke Toxic Effects in Regenerating Liver.

The following control study assessed the time course for recovery from partial hepatectomy (Table 4). Partial liver resection is commonly used in the treatment of liver tumors. The time course of recovery from partial hepatectomy was assessed by the release of hepatic enzymes (serum alanine aminotransferase (ALT); serum aspartate aminotransferase (AST)) (see, e.g., Nathwani, et al. (2005) Hepatology 41:380-383; Clavien, et al. (2003) Ann Surg. 238:843-850). Serum enzyme levels were found to reach a basal level by t=3 days after the partial hepatectomy (Table 4).

TABLE 4
Mean serum enzyme levels at intervals after partial hepatectomy.

Day

| | 0 | 1 | 2 | 3 | 4 | 5 |
|---|---|---|---|---|---|---|
| Mean | 4401 | 939 | 209 | 110 | 94 | 171 |
| AST | | | | | | |
| Mean | 5228 | 952 | 198 | 130 | 41 | 58 |
| ALT | | | | | | |

The following control study demonstrated that the LD₅₀ for Listeria is the same, or similar, in normal mice and in hemispleen mice. In normal mice, the LD₅₀ for Listeria ΔactA was 1.0×10⁸ bacteria (also expressed using the following terminology: 1.0e8), and for Listeria ΔactAΔinlB was 2 to 5×10⁸ bacteria. In the hemispleen mice, the LD₅₀ for Listeria ΔactA was 1.23×10⁸ bacteria (also expressed using the following terminology: 1.23e8), and for Listeria ΔactAΔinlB was greater than 1.49×10⁸ bacteria (Table 5).

Naive mice, or mice receiving a partial hepatectomy were titrated with Listeria ΔactA or with Listeria ΔactAΔinlB, to determine if the partial hepatectomy influenced Listeria toxicity (Table 5). At t=0 days, mice received no surgery, or a partial hepatectomy (about 40%). At t=3 days, all mice received the indicated amount of Listeria (Table 5).

TABLE 5
Survival of naive mice and partial hepatectomized
mice after Listeria challenge.

| | | Partial | Naive mice |
|---|---|---|---|
| | LD₅₀ | hepatectomized | (no partial |
| Listeria administered. | (Listeria dose) | mice | hepatectomy) |
| Listeria ΔactA | 5.52 × 10⁸ | 2/2 | 0/3 |
| Listeria ΔactA | 1.44 × 10⁸ | 3/3 | 0/3 |
| Listeria ΔactA | 6.29 × 10⁷ | 2/3 | 1/3 |
| Listeria ΔactA | 8.30 × 10⁶ | 0/3 | 3/3 |
| Listeria ΔactAΔinlB | 6.67 × 10⁸ | 2/2 | 0/3 |
| Listeria ΔactAΔinlB | 1.12 × 10⁸ | 3/3 | 0/3 |
| Listeria ΔactAΔinlB | 5.57 × 10⁷ | 1/3 | 0/3 |
| Listeria ΔactAΔinlB | 1.12 × 10⁷ | 1/2 | 3/3 |

V. Administration of Listeria Activates Immune Cells in the Liver.

L. monocytogenes was administered to mice followed by assessment of the in vivo modulation of immune response, as determined by extracting the immune cells from the liver and spleen, and by identifying these cells. Except where indicated, Listeria was administered to mice at t=0 hours, followed by sacrifice at t=24 hours. In the time course experiments, where indicated, mice were sacrificed at t=24 hours or at t=48 hours. Livers and spleens were homogenized and dispersed. Cells were washed twice with Hanks Balanced Salt Solution (HBSS), then blocked for 15 min on ice with 4% HAB and anti-CD16/32 antibody. HAB is “Hanks Azide Buffer,” which contains 1% bovine serum albumin, 0.1% sodium azide, and 1 mM EDTA.

Antibody specific for the cell marker of interest was added, and cells incubated 30 minutes on ice. Cells were washed three times, then suspended in 1% formaldehyde, and analyzed by Fluorescence Activated Cell Sorting (FACS). L. monocytogenes (ΔactA or ΔactAΔinlB) was administered at an amount equivalent to zero LD₅₀ (HBSS only); 0.01 LD₅₀; 0.1 LD₅₀; or 0.25 LD₅₀. Table 6 discloses some of the parameters studied in the following experiments.

TABLE 6
Parameters measured in immune cells extracted from liver and spleen.

% NK cells compared to total leukocytes.
NK cell activation (CD69)
% NKT cells compared to total leukocytes.
NKT cell activation (CD69)
% T cells compared to total leukocytes.
% CD8⁺ T cells compared to total leukocytes.
% CD4⁺ T cells compared to total leukocytes.
CD4⁺ T cell activation (CD69)
CD8⁺ T cell activation (CD69)
% neutrophils compared to total leukocytes.
% of CD4⁺ T cells that are CD4⁺CD25⁺ T cells.
Time courses for changes in the % of NK cells and neutrophils.

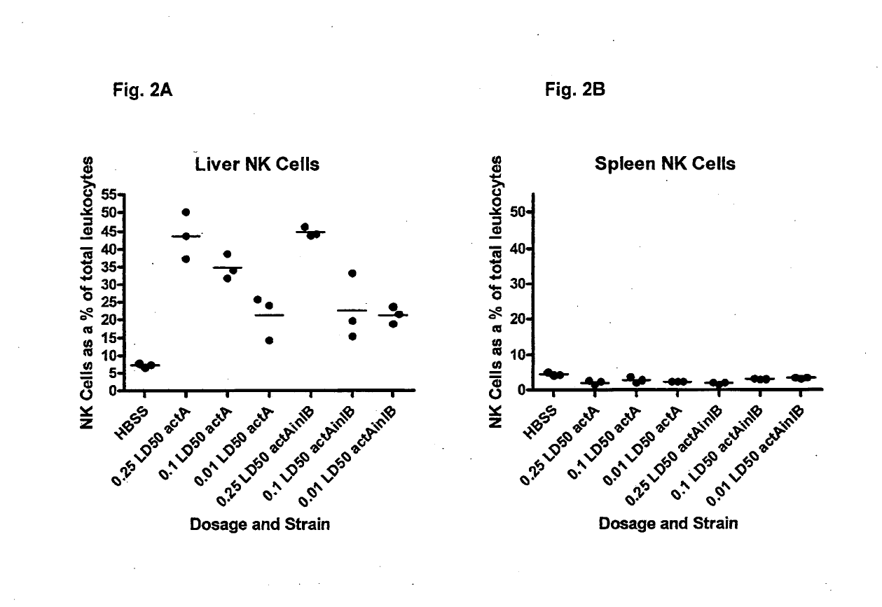

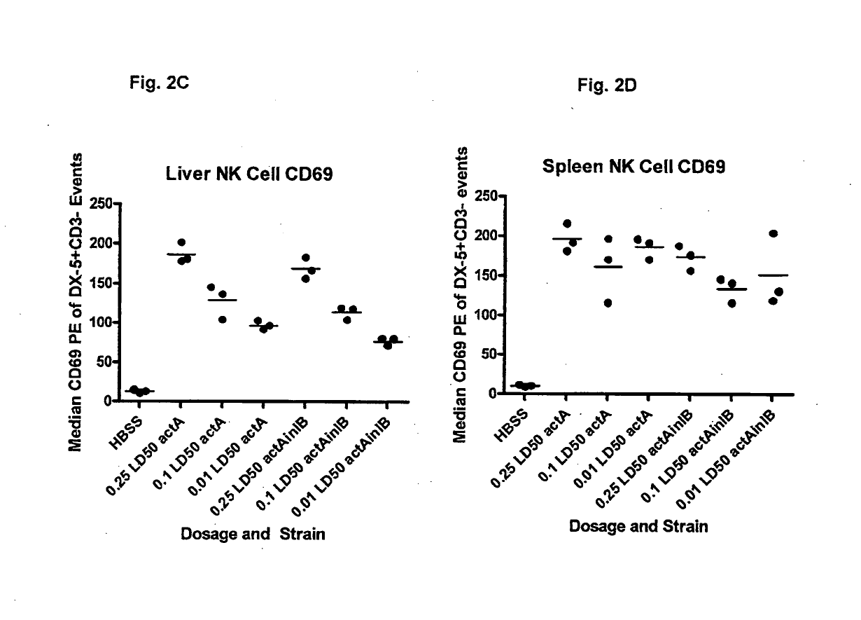

The results were as follows (FIGS. 2A to 2D). The percent of NK cells (% of total leukocytes) increased in the liver, with increasing doses of Listeria. With increasing doses, the percent of total leukocytes that was NK cells increased from about 7% (only HBSS administered, no bacteria), about 20% (dose of 0.01 LD₅₀); about 35% (0.1 LD₅₀); and about 44% (0.25 LD₅₀) (FIG. 2A). NK cell activation in the liver, as assessed by mean fluorescence intensity of expressed CD69, increased from about 10 (arbitrary units where value in absence of cells is zero) (HBSS only, no bacteria); to about 100 (0.01 LD₅₀); to about 130 (0.1 LD₅₀), to about 190 (0.25 LD₅₀) (FIG. 2C). The designation “only HBSS administered” means that no bacteria were administered, and that the data point represents a control value. FIGS. 2B and 2D disclose spleen data.

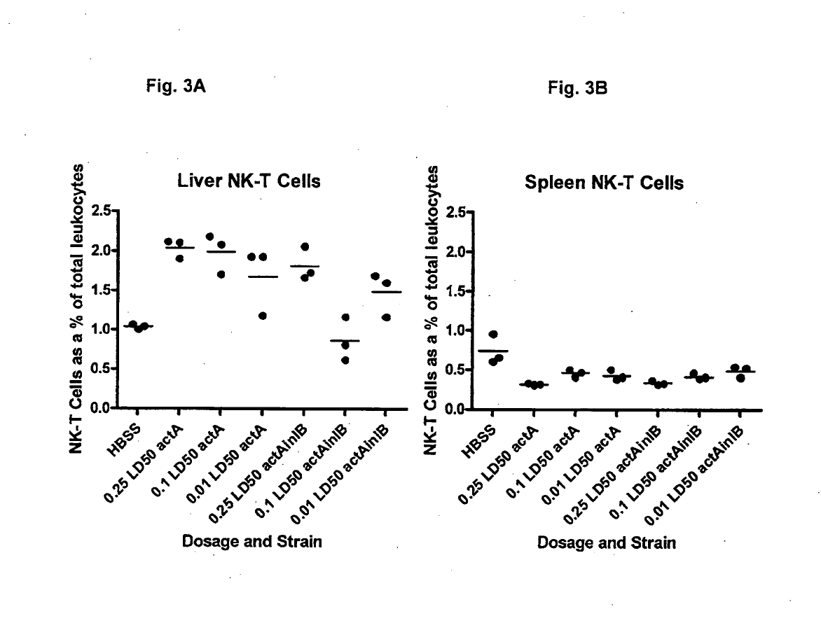

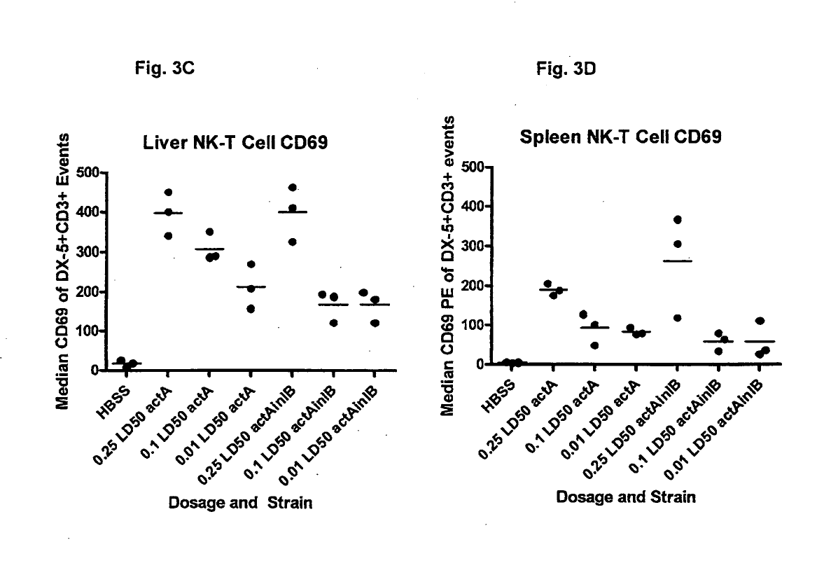

The following concerns NKT cells. Activation of NKT cells in the liver increased with administration of Listeria, where activation after giving Listeria ΔactA was about 5 (HBSS only, no bacteria); 200 (0.01 LD₅₀); 300 (0.1 LD₅₀); and 400 (0.25 LD₅₀) (FIGS. 3A and 3C). After administering the other deletion mutant of Listeria (Listeria ΔactAΔinlB), maximal activation was also found with administration of 0.25 LD₅₀. (The term “maximal activation” means that maximal activation found with the indicated doses, and does not necessarily mean that higher doses cannot generate even higher states of activation.) (FIGS. 3A and 3C). FIGS. 3B and 3D reveal spleen data.

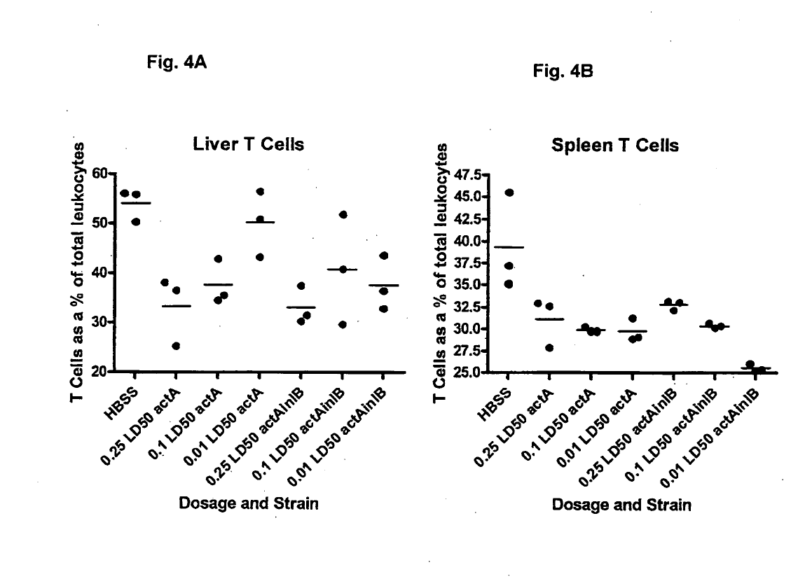

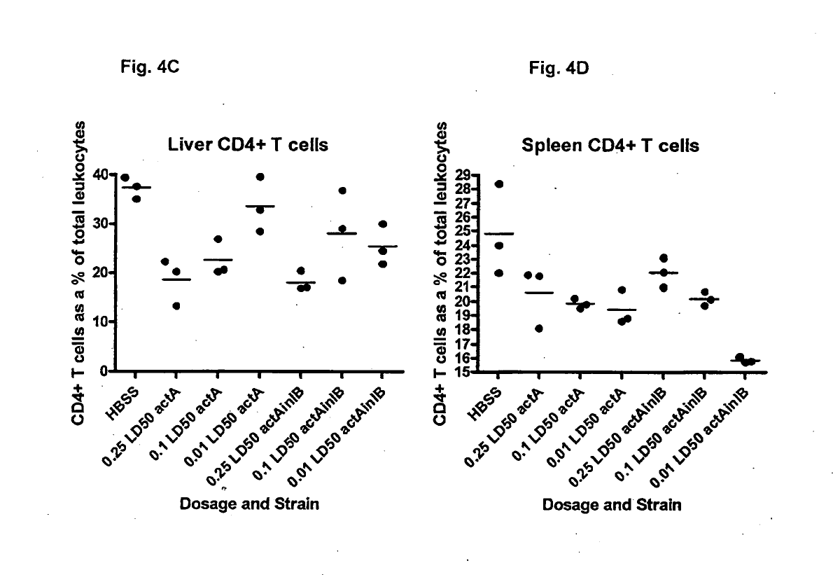

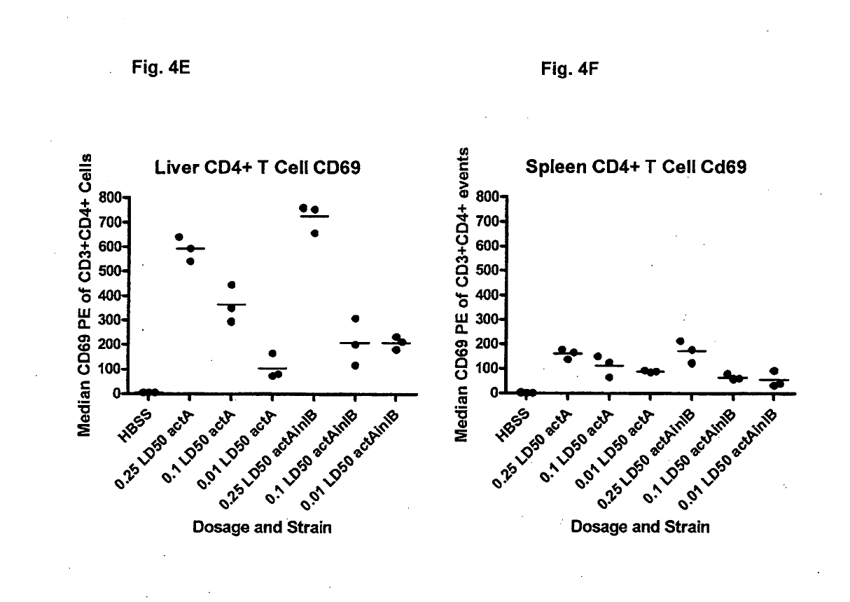

FIGS. 4A and 4B discloses results with total liver T cells.

The following concerns CD4⁺ T cells in the liver (FIGS. 4C to 4F). After administering Listeria ΔactA, activation was about 0 (HBSS only, no bacteria), 100 (0.01 LD₅₀), 350 (0.1 LD₅₀), and 600 (0.25 LD₅₀). With administering the other Listeria strain, Listeria ΔactAΔinlB, maximal activation also occurred at the highest dose (FIGS. 4A, C, and E). FIGS. 4B, D, and F disclose spleen data.

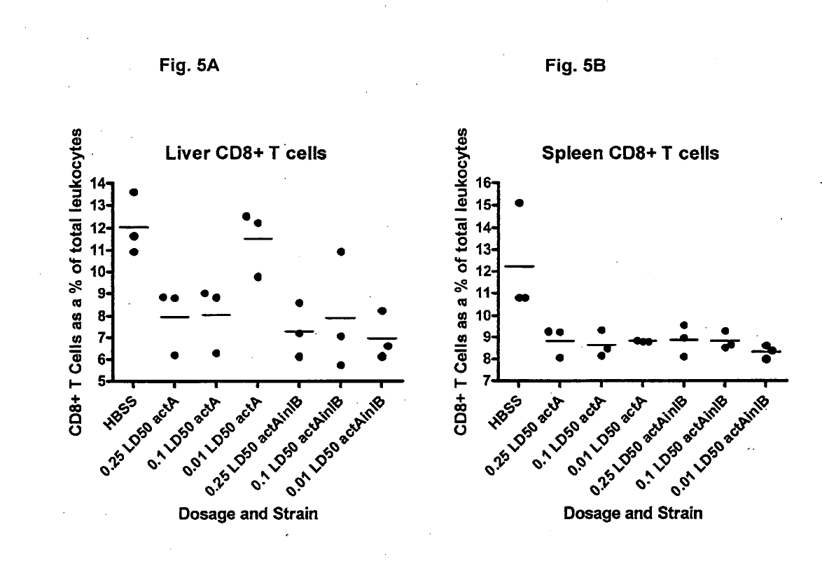

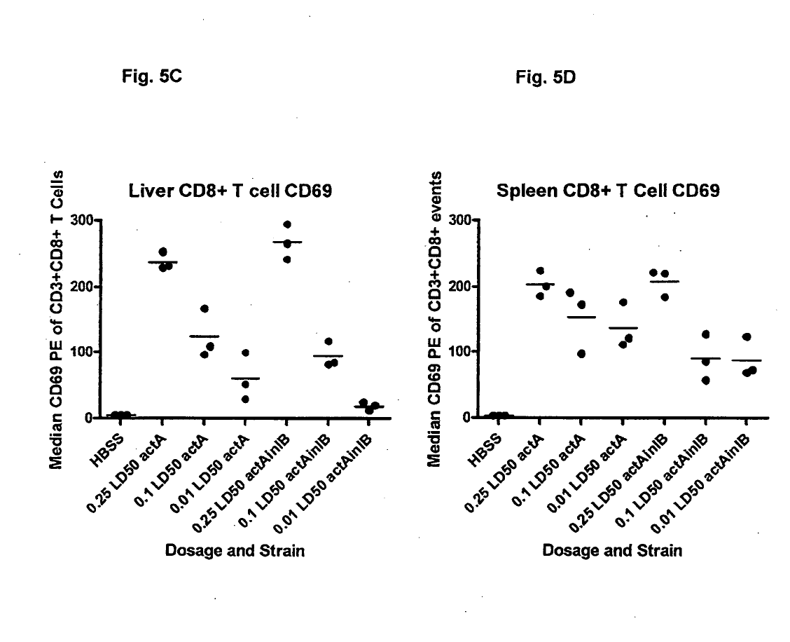

The following concerns CD8⁺ T cells in the liver (FIGS. 5A to 5D). Activation of CD8⁺ T cells in liver with Listeria ΔactA was about 0 (HBSS only, no bacteria), 60 (0.01 LD₅₀), 120 (0.1 LD₅₀), and 230 (0.25 LD₅₀). Administration of the other strain of Listeria, Listeria ΔactAΔinlB, produced a similar activation profile (FIGS. 5A and 5C. FIGS. 5B and 5D show spleen data.

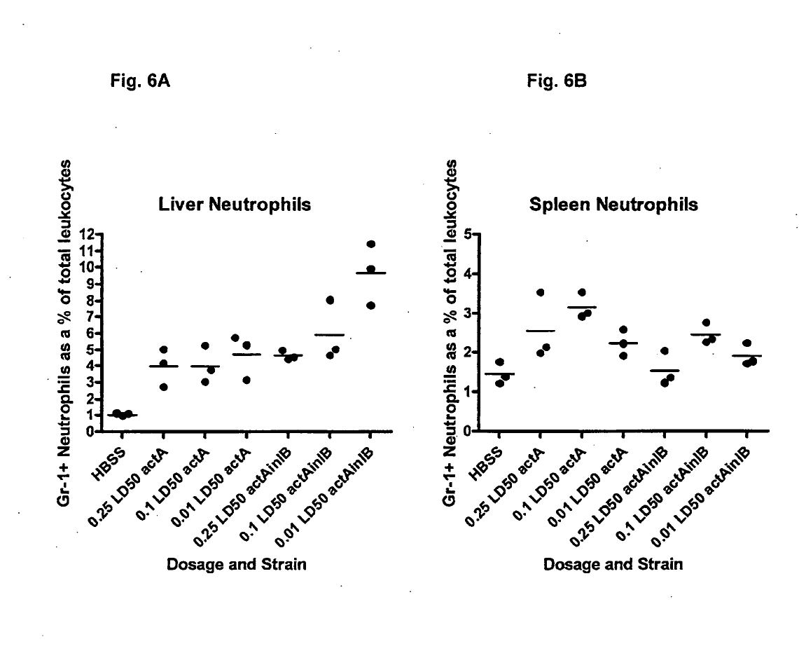

The following concerns neutrophils (FIGS. 6A and 6B). Liver neutrophils increased from about 1% (HBSS only, no bacteria) to about 4-5%, with all three doses of administered Listeria ΔactA. With administered Listeria ΔactAΔinlB, the neutrophils accounted for about 5-10% of the total leukocytes (FIG. 6A). FIG. 6B shows spleen data.

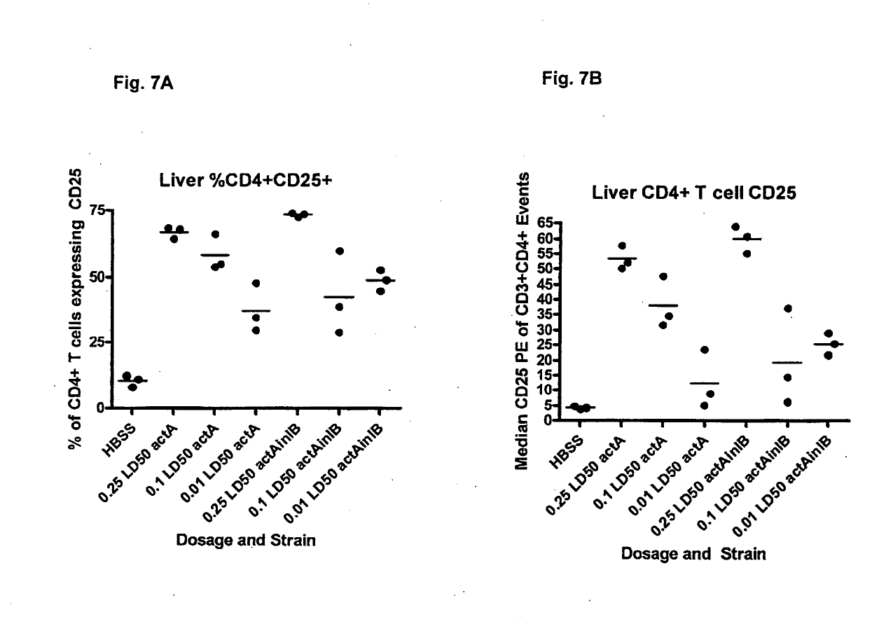

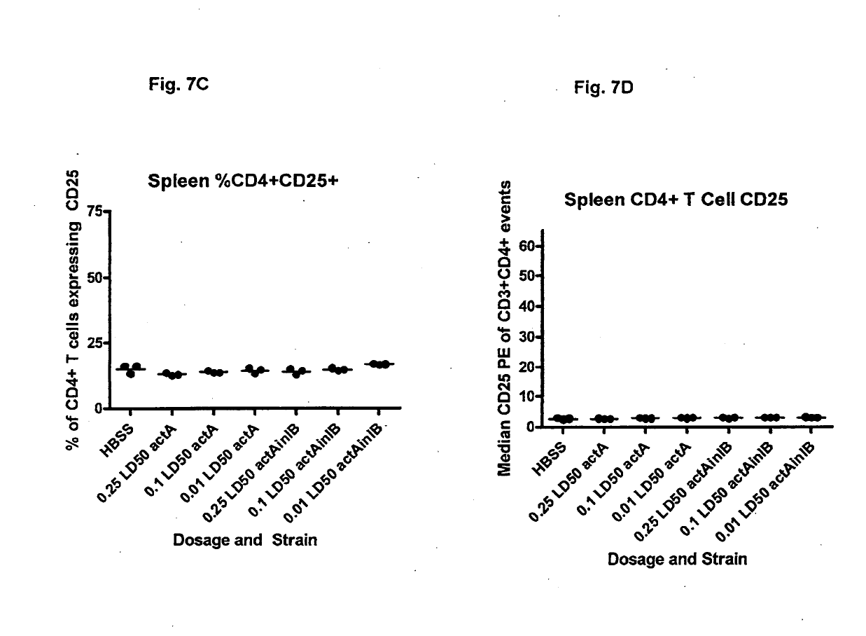

The presence of CD4⁺ T cells expressing CD25 was also measured, as was the mean amount of CD25 expressed on individual cells (FIGS. 7A to 7D). CD25 expression was measured after administering Listeria ΔactA or Listeria ΔactAΔinlB. Data from liver CD4⁺ T cells and spleen CD4⁺ T cells are shown (FIGS. 7A to 7D).

The following concerns dendritic cells, that is, CD8⁺ alpha negative dendritic cells. Control mice were administered HBSS, while experimental mice were given L. monocytogenes ΔactA (expressing ova). The percentage of these dendritic cells, compared to all splenocytes, was determined over the course of several days. A goal of the present work was to determine the effect of administered Listeria on this dendritic cell population. (For assessing this goal, it is not expected to be relevant if the Listeria expresses ova.) Maturation of the DCs was also measured, as assessed by the markers CD80 and CD86. CD80 and CD86 are DC maturation markers (Gerosa, et al. (2005) J. Immunol. 174:727-734; Kubo, et al. (2004) J. Immunol. 173:7249-7258). For these dendritic cells, control treatment (HBSS salt solution) resulted in relatively constant percentage values (2.0% (day 1); 1.9% (day 2); 1.9% (day 4); 1.6% (day 7)). Experimental treatment (Listeria ΔactA ova) resulted in marked increases in the percent of this type of dendritic cell (3.4% (day 1); 7.3% (day 2); 2.0% (day 4); 1.9% (day 7)). Regarding the CD80 and CD86 markers, the following results were found. Control treatment (HBSS salt solution) of mice resulted in the following CD80 relative expression values for DCs isolated from the spleen: 105 (day 1); 78 (day 2); 91 (day 3), 53 (day 4). Experimental treatment (Listeria ΔactA ova) resulted in dramatic increases in these CD80 expression expression values, that is, on days one and two: 372 (day 1); 298 (day 2); 98 (day 3); 102 (day 7). The following data concern the other marker, CD86. Control treatment (HBSS salt solution) resulted in these CD86 expression values: 31 (day 1); 18 (day 2); 30 (day 4); and 30 (day 7). Experimental treatment provoked a dramatic increase in CD86 expression on days one and two: 257 (day 1); 80 (day 2); 38 (day 4); and 24 (day 7).

The above results, which concern populations of dendritic cells, and the maturation of dendritic cells, are important for immune response to tumors and infections, for a number of reasons. To give two examples, an administered Listeria that enhances DC populations or DC maturation is expected to enhance NK cell function and also to relieve the suppressive effects of regulatory T cells (see, e.g., Gerosa, et al. (2005) J. Immunol. 174:727-734; Kubo, et al. (2004) J. Immunol. 173:7249-7258).

VI. Time Course Studies with Administration of Attenuated Listeria, with Data Disclosing Stimulation of NK Cells and Neutrophils.

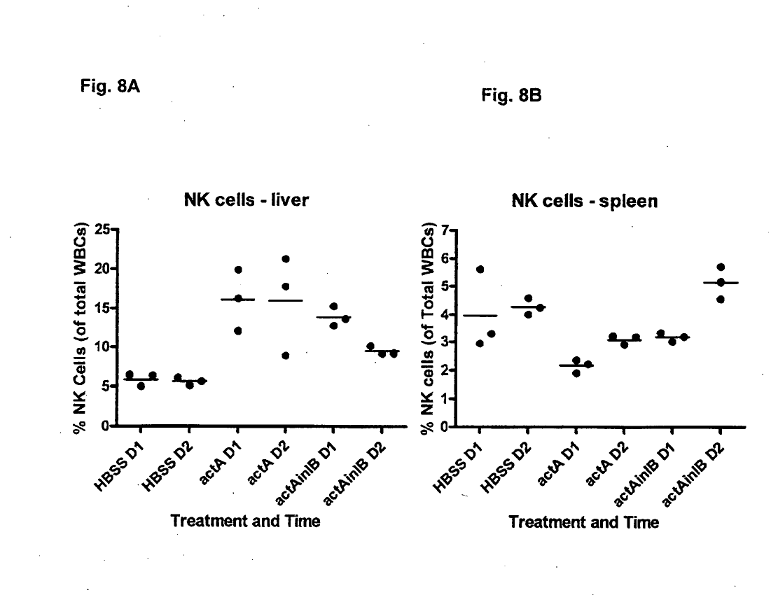

Mice were administered HBSS, Listeria ΔactA, or Listeria ΔactAΔinlB, and sacrificed 24 hours later (D1) or 48 hours later (D2), followed by determinations of the number of NK cells or neutrophils, as compared to the total number of leukocytes. Data from analysis of leukocytes recovered from the liver demonstrated that the percent of leukocytes occurring as NK cells was the same on both days (about 6%) with doses of HBSS, the same on both days (about 16%) with doses of Listeria ΔactA, and somewhat greater at t=24 hours (14%) than at t=48 hours (10%) after doses of the other Listerial strain, Listeria ΔactAΔinlB (FIG. 8A). FIG. 8B discloses spleen data.

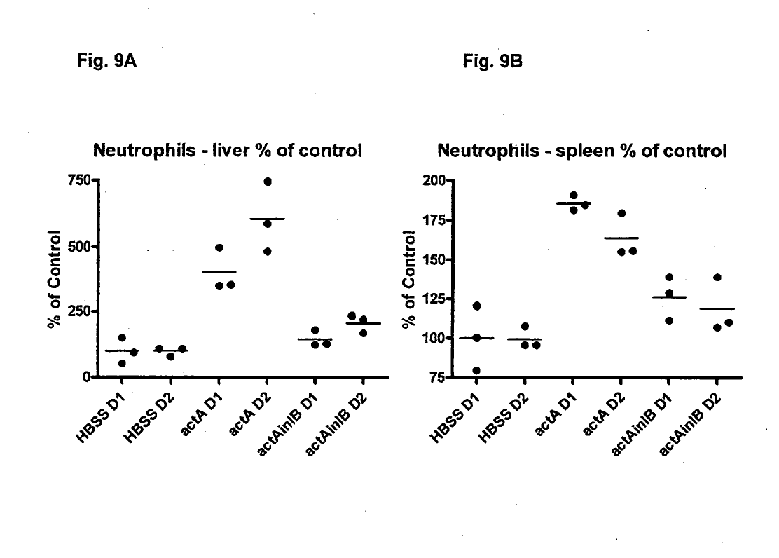

Data from the analysis of neutrophils recovered from the liver demonstrated that in HBSS-administered mice, neutrophils accounted for about 0.2 to 0.8% of liver leukocytes. One day after administering Listeria ΔactA, neutrophils accounted for about 3% of the liver leukocytes, with lesser percent values found under the other conditions of the experiment (FIG. 9A). FIG. 9B discloses spleen data.

A separate study revealed that administering Lm ΔactAΔinlB to mice resulted in the in vivo generation of activated NK cells, where the activated NK cells showed an enhanced ability to kill YAC-1 cells, in vitro. YAC-1 cells are conventionally used as an NK cell target. C57BL/6 mice were injected with 3×10⁷ cfu of Lm ΔactAΔinlB, or with a negative control vehicle. After a delay of 24 h, 48 h, or 72 h, lymphocytes were harvested from the liver or spleen, and the harvested lymphocytes (contains NK cells) were mixed with chromium-labeled YAC-1 cells (the target cells), and then incubated for 4 h. With lymphocytes harvested at the 48 h time point, for example, liver NK cells produced about 50% lysis of the target cells (whereas only 3% target cell lysis occurred where lymphocytes were from vehicle-treated mice). With lymphocytes harvested at the 48 h time point, spleen NK cells produced about 30% lysis of the target cells (whereas only 7% lysis of target cells occurred where lymphocytes were from vehicle-treated mice). Thus, the methods of the invention provide for administering Lm for activating and/or increasing hepatic levels of NK cells, where the NK cells are effective at lysing target cells.

VII. Administering Listeria Increases Numbers of Immune Cells in the Liver (Time Course Studies).

The following discloses the time course of accumulation of various immune cells in the liver following administration of Listeria ΔactA. Concurrent work illustrates the influence, on immune cell accumulation, produced by administering only tumor cells engineered to express GM-CSF (GVAX), or produced by administering Listeria ΔactA together with GVAX.

Balb/c mice were treated under the following conditions, followed by measuring the number of various immune cells in the liver. The treatments were:

(1) Naive mice (not administered any tumor cells);

(2) No treatment (NT) mice (administered tumor cells but not treated with Listeria and not treated with GVAX);

(3) Administered tumor cells and GVAX;

(4) Administered tumor cells and Listeria ΔactA (Lm-actA); and

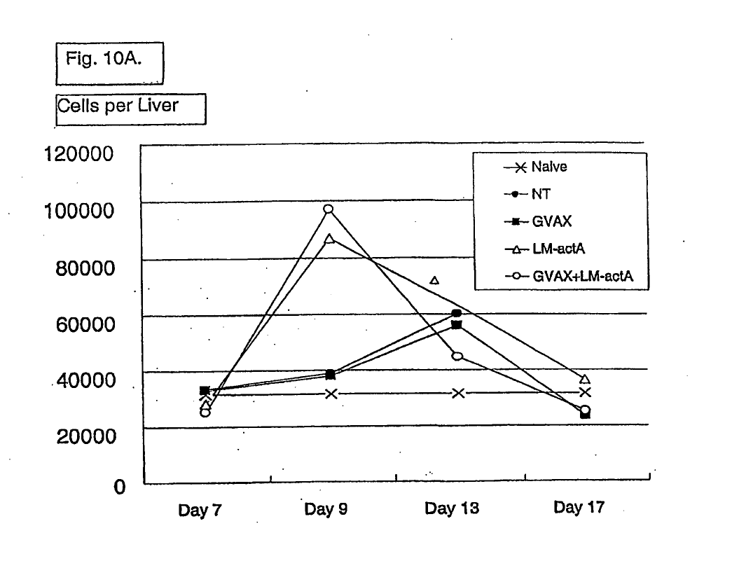

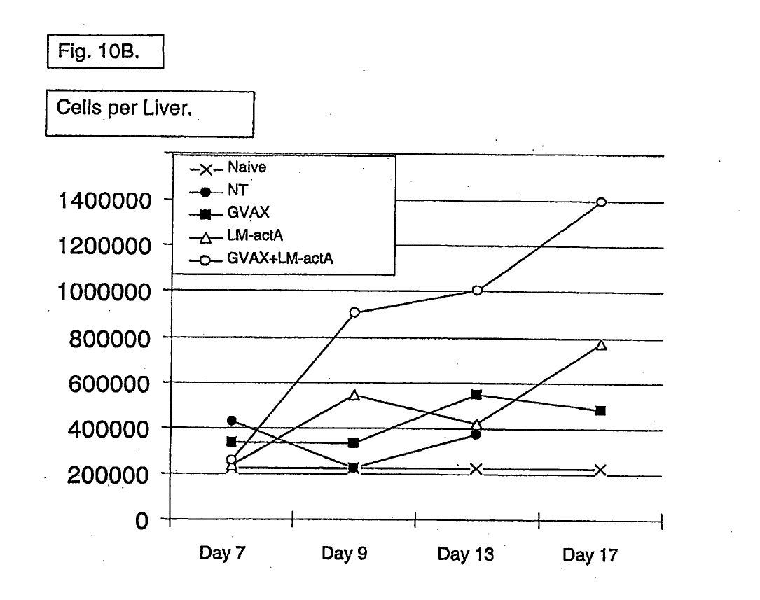

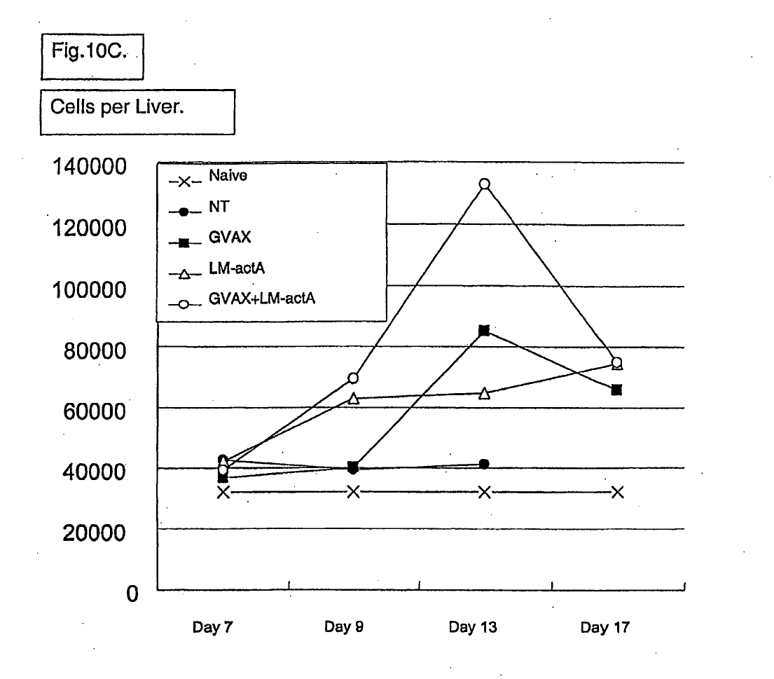

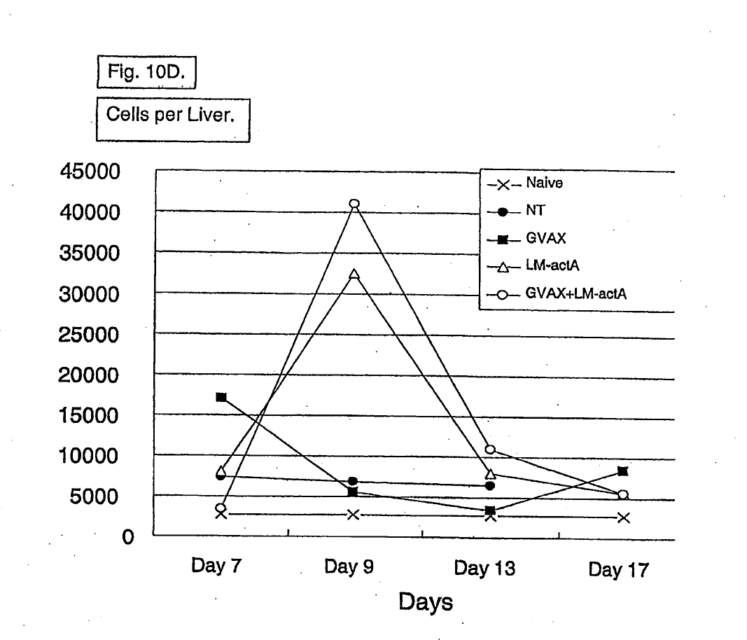

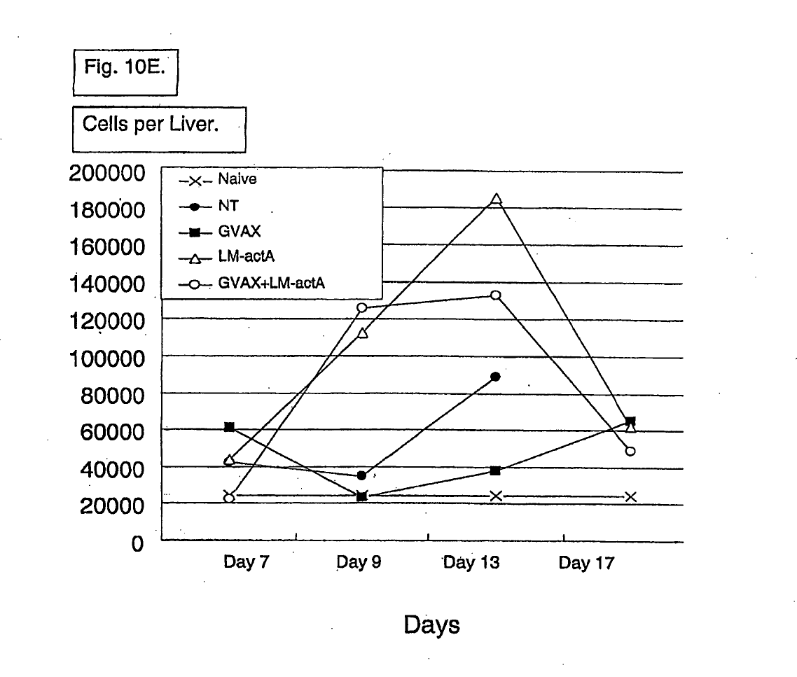

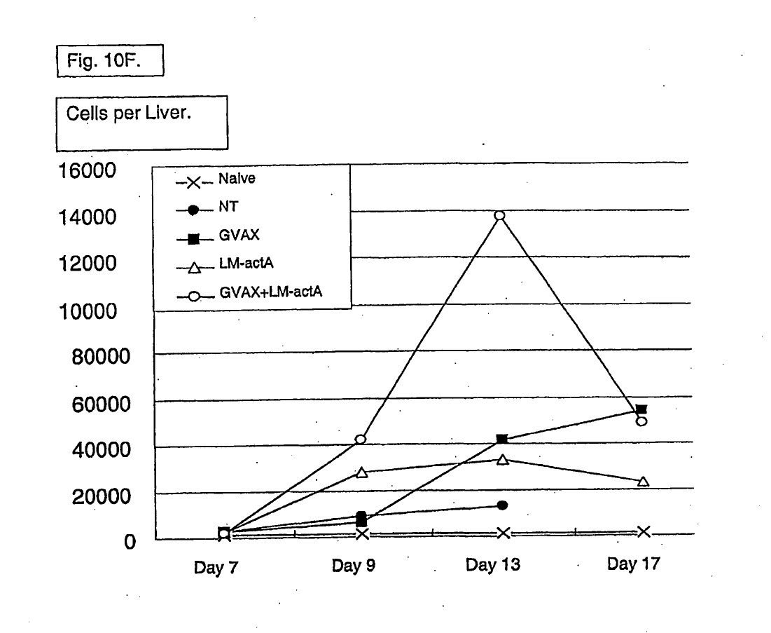

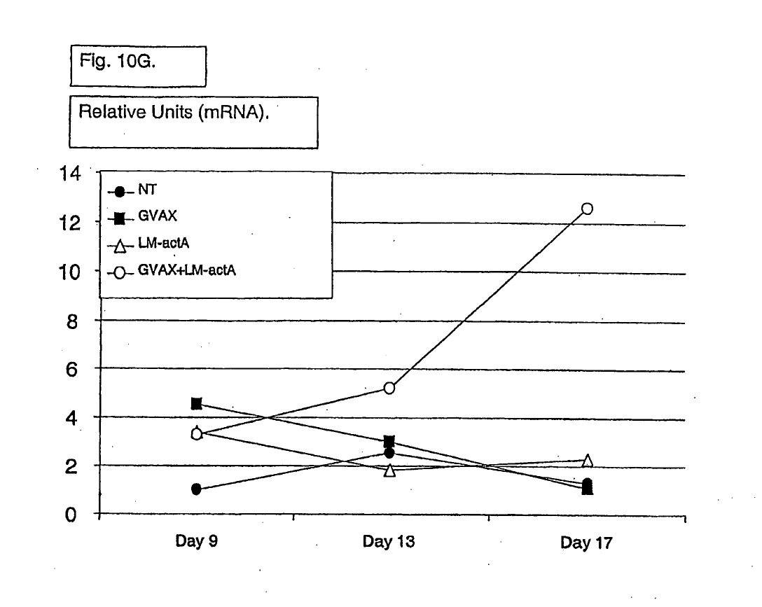

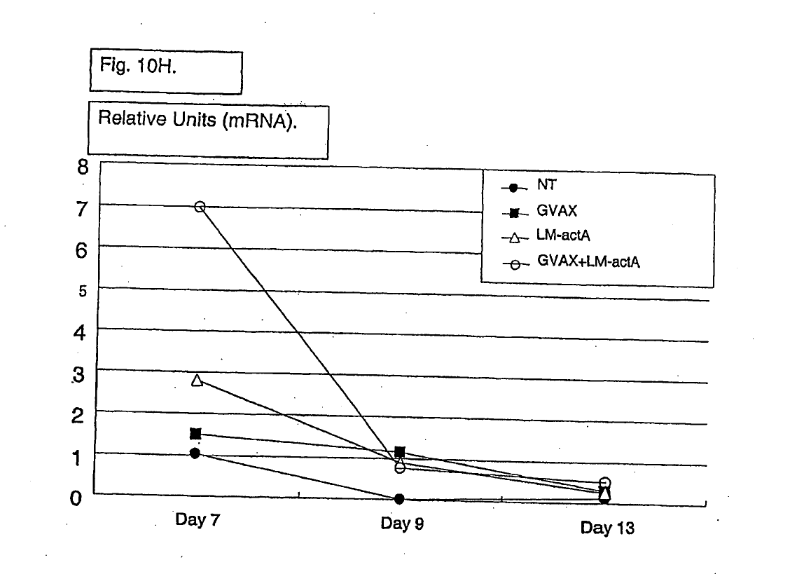

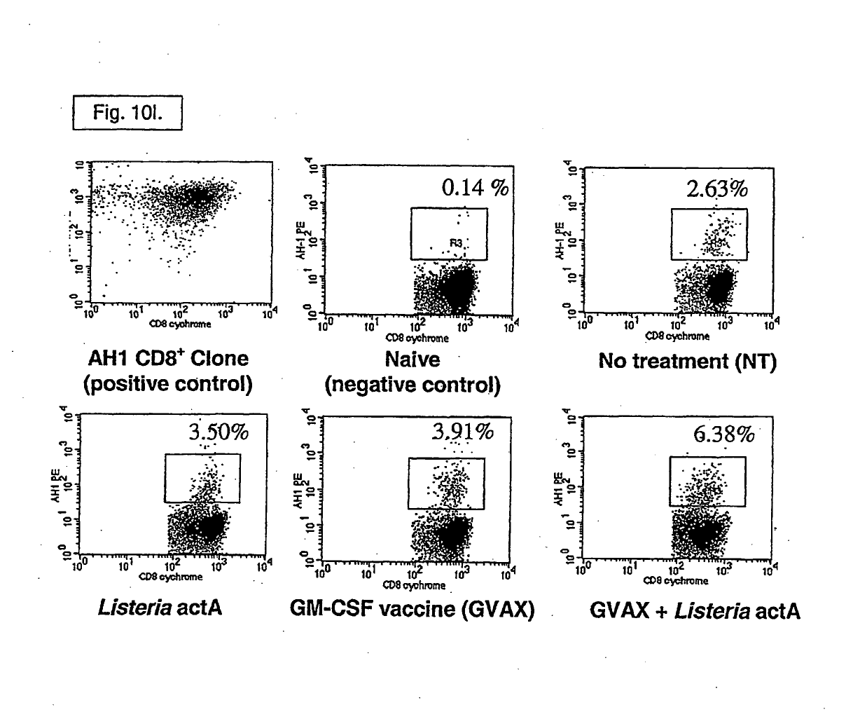

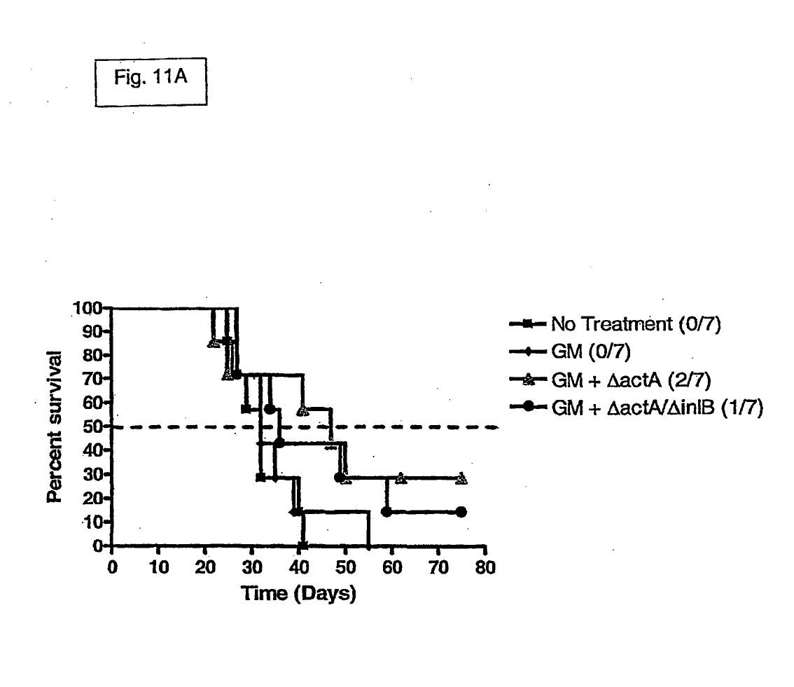

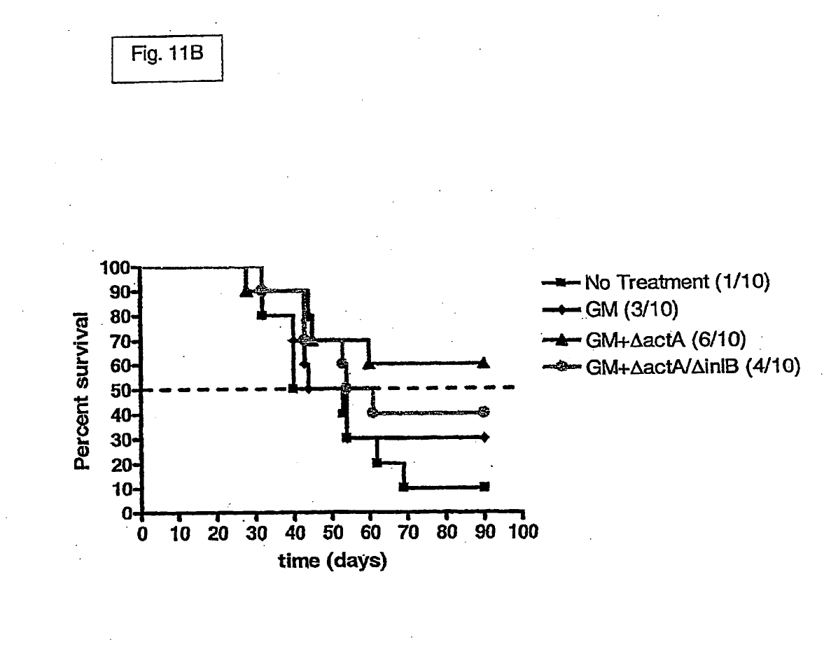

(5) Administered tumor cells, GVAX, and Listeria ΔactA (Lm-actA).

Where Listeria ΔactA was given, the number of administered bacteria was 1×10⁷ CFU. The immune cells that were identified and counted were: NK cells (FIG. 10A); NKT cells (FIG. 10B); CD8⁺ T cells (FIG. 10C); plasmacytoid dendritic cells (plasmacytoid DCs) (FIG. 10D); myeloid DCs (FIG. 10E); and tumor specific CD8⁺ T cells (FIG. 10F). The activation state of tumor specific CD8⁺ T cells (in the liver) was assessed by measuring expression of interferon-gamma (IFNgamma mRNA) (FIG. 10G). The activation state of NK cells (in the liver) was also assessed, where activation was assessed by measuring IFNgamma mRNA (FIG. 10H).

The results were as follows. Regarding the general baseline population range, the dendritic cells (DCs) in the liver tended occur at the lowest population ranges while NK cells, NKT cells, and CD8⁺ T cells tended to occur at the highest population ranges. The baselines for all cell types was constant for the naive mice (FIGS. 10A-10F). When Listeria alone was administered to tumor-bearing mice, the NK cell population showed a peak at about t=9 days (FIG. 10A); the NKT cell population showed an increasing trend up to at least 17 days (FIG. 10B); CD8⁺ T cells showed a steady increasing trend up to at least 17 days (FIG. 10C); plasmacytoid DCs showed a peak at about t=9 days (FIG. 10D); the myeloid DC population peaked at about t=13 days (FIG. 10E); while tumor-specific CD8⁺ T cells peaked at about t=13 days (FIG. 10F).

GVAX alone increased the populations of all of the immune cells (FIGS. 10A- 10F). Listeria in combination with GVAX revealed additive effects, or synergic effects, in the cases of NKT cells (FIG. 10B); CD8⁺ T cells (FIG. 10C); plasmacytoid DCs (FIG. 10D); and tumor specific CD8⁺ T cells (FIG. 10F).

The activation state of a number of immune cells was assessed, where assessment was by assays of interferon-gamma (IFN-gamma) mRNA. Assays for IFN-gamma mRNA expressed by tumor specific CD8⁺ T cells revealed that the greatest increase in expression occurred with administration of both Listeria and GVAX to the mice (FIG. 10G). Assays for IFN-gamma mRNA expressed by NK cells also showed that the greatest increase in expression occurred with administration of both Listeria and GVAX to the mice (FIG. 10H). With regard to the mice receiving both Listeria and GVAX, a difference was noted in following IFN-gamma expression by the tumor specific CD8⁺ T cells and NK cells, namely that expression by the CD8⁺ T cells was highest at later time periods, while expression by the NK cells was highest at the earlier time periods (FIGS. 10G and H).

The following concerns FIG. 10I. FIG. 10I shows analysis of CD8⁺ T cells taken from livers of CT26 tumor cell-innoculated mice, where the mice had also been administered, e.g., various therapeutic agents. The therapeutic treatments, including controls, included no therapeutic treatment (NT); L. monocytogenes ΔactA; GM-CSF vaccine only (GVAX); and L. monocytogenes ΔactA plus GVAX. With no therapeutic treatment (NT), the percent of tumor antigen-specific CD8⁺ T cells was 2.63%.

The results were as follows. With Listeria only, the percent of tumor antigen-specific CD8⁺ T cells was higher (3.5%); with GVAX only, and the percent of tumor antigen-specific CD8⁺ T cells was also higher (3.91%). But with Listeria plus GVAX the percent of expression of tumor antigen-specific CD8⁺ T cells was much higher (6.38%), demonstrating synergy between the Listeria and the GM-CSF vaccine (FIG. 10I).

In detail, the figure illustrates analysis of tumor-specific CD8⁺ T cells that infiltrate the liver in treated mice with hepatic metastases. Specific flow cytometry plots on cells isolated from the livers of mice sacrificed on day 13 and stained with anti-CD8 (FITC) and L^{d}-AH1 tetramers (cychrome) are shown. Note that AH1 is the immunodominant MHC class I-restricted tumor antigen recognized by CT-26-specific CD8⁺ T cells. The study involved positive and negative controls (AH1-specific CD8⁺ T cell clone as a positive control; and hepatic CD8⁺ cells from naive non-tumor-bearing mice as a negative control). The data represent the results from the pooled and processed livers of three mice. Treatment with both CT-26/GM-CSF and Listeria ΔactA resulted in the highest level of hepatic AH1-specific CD8⁺ T cells.

VIII. Administering an Attenuated Tumor Cell Line that Expresses GM-CSF Increases Survival to Tumors, while Administering that Tumor Cell Line with Listeria ΔactA or Listeria ΔactAΔinlB Further Increases Survival to Tumors.

Tumor bearing mice were treated by administering: (1) Salt water only (HBSS); (2) A vaccine comprising a tumor cell line secreting a cytokine (CT26 cells expressing the cytokine GM-CSF) (GM-CSF vaccine); (3) The vaccine plus Listeria ΔactA; or (4) The vaccine plus Listeria ΔactAΔinlB.

Tumor cells (1×10⁵ CT26 cells) in 0.05 ml HBSS were administered into the hemispleen, followed by a flush of 0.25 ml HBSS. Irradiated GM-CSF expressing CT26 cells (1×10⁶ cells) (also known as “vaccine”) were administered in 0.30 ml of HBSS, with 0.10 ml injection per site (subcutaneously; s.c.). Listeria was administered in amount equivalent to 0.1 LD₅₀, where administration was in 0.20 ml HBSS (i.p.) or in 0.10 ml HBSS (intravenously; i.v.). The time line for the various administrations during the course of the experiment was as follows: tumor (t=0 days); vaccine (t=3 days); vaccine plus Listeria (t=6 days); vaccine (t=13 days); and vaccine (t=21 days). Conditions of the experiment included no treatment (-▪-; filled squares); vaccine only (-♦-; diamonds); vaccine plus Listeria ΔactA (-▴-; filled triangles); and vaccine plus Listeria ΔactAΔinlB (-•-; filled circles). CT26 tumor cells were administered at t=day zero, while GM-CSF vaccine was given at t=3 days, and Listeria provided at t=6 days. For FIGS. 11A and 11B, the Listeria dose was 1×10⁷ CFU.

FIG. 11A discloses the percent survival of the mice versus time (days) during the study. The results demonstrated that in the “no treatment” group, there were zero survivors by t=40 days, and that survival was somewhat greater in the vaccine only group, with zero survivors by t=55 days. The vaccine plus Listeria groups resulted in markedly enhanced survival, with about 28% survival at t=48 days in both vaccine plus Listeria ΔactA group and vaccine plus Listeria ΔactAΔinlB group, while at t=75 days, 28% survival was found in the vaccine plus Listeria ΔactA group, and about 15% survival in the vaccine plus Listeria ΔactAΔinlB group (FIG. 11A). FIG. 11B shows data from a repeated trial of the same experiment as above. Again, mice receiving no treatment showed the poorest survival, with only one mouse surviving at t=90 days. Again, mice receiving the GM-CSF vaccine with Listeria showed the best survival. Here, 6 out of 10 mice receiving the GM-CSF vaccine plus Listeria ΔactA still survived at t=90 days, and 4 out of 10 mice receiving the GM-CSF vaccine plus Listeria ΔactAΔinlB survived at t=90 days (FIG. 11B).

The present invention provides a method comprising administering an attenuated Listeria (e.g., L. monocytogenes ΔactA or L. monocytogenes ΔactAΔinlB), with attenuated tumor cells (e.g. irradiated metastatic cells), where the cells had been engineered to express a cytokine, e.g., GM-CSF. In the present invention, the Listeria are not engineered to comprise any nucleic acid encoding any heterologous antigen, e.g., a tumor or infectious agent antigen. In another aspect of the present invention, the Listeria are engineered to comprise a nucleic acid encoding a heterologous antigen.

IX. Cyclophosphamide Increases Survival to Tumors.

Administering cyclophosphamide (CTX) increased survival of mice bearing tumors under each of these three conditions:

(1) Mice treated with GM-CSF vaccine only;

(2) Mice treated with GM-CSF vaccine plus Listeria ΔactA;

(3) Mice treated with GM-CSF vaccine plus Listeria ΔactAΔinlB.

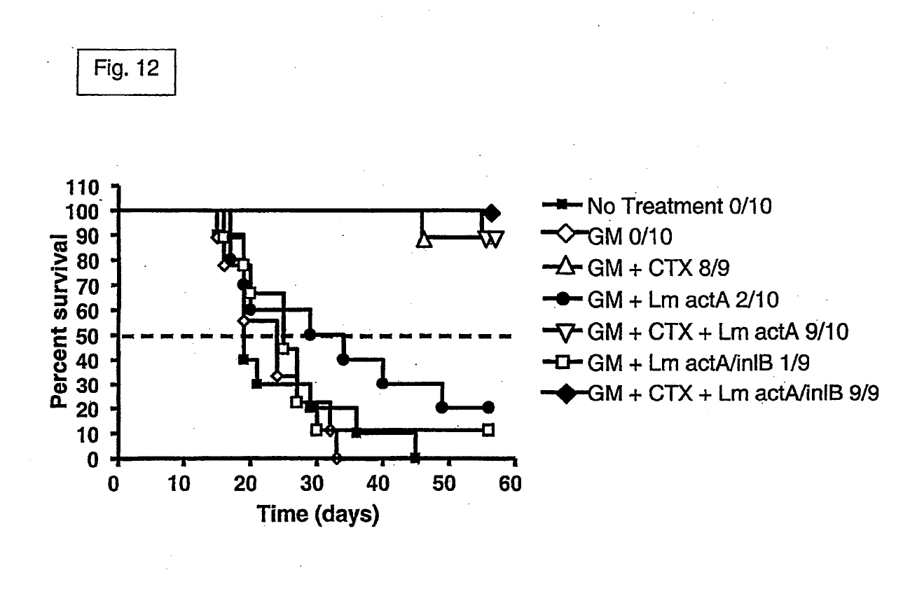

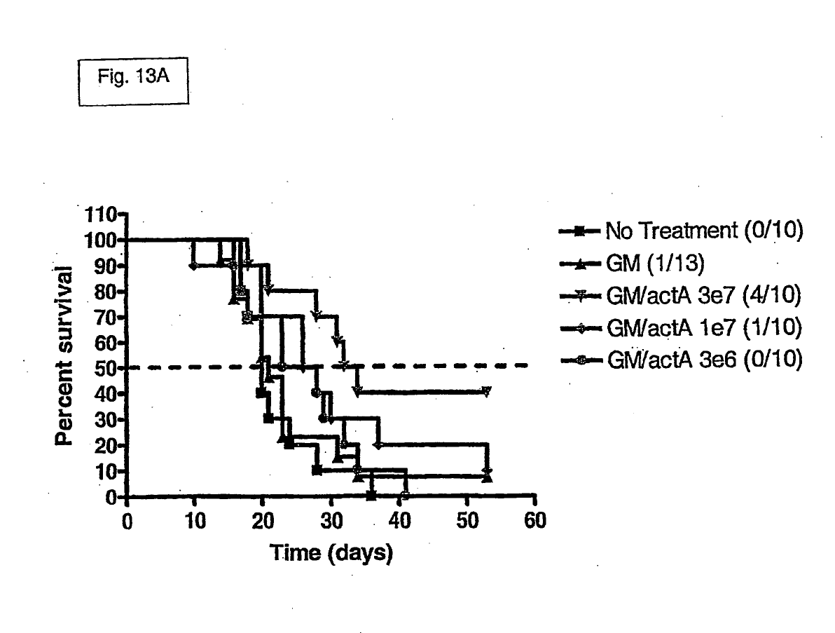

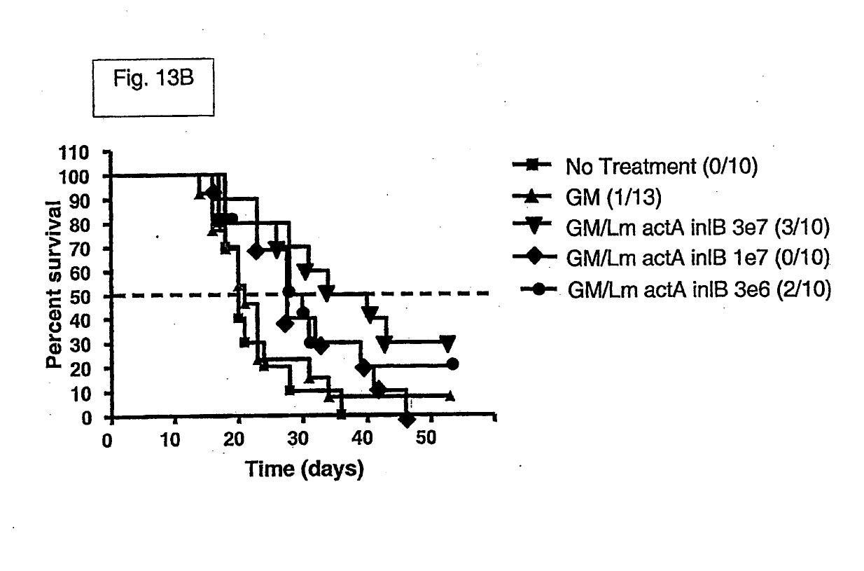

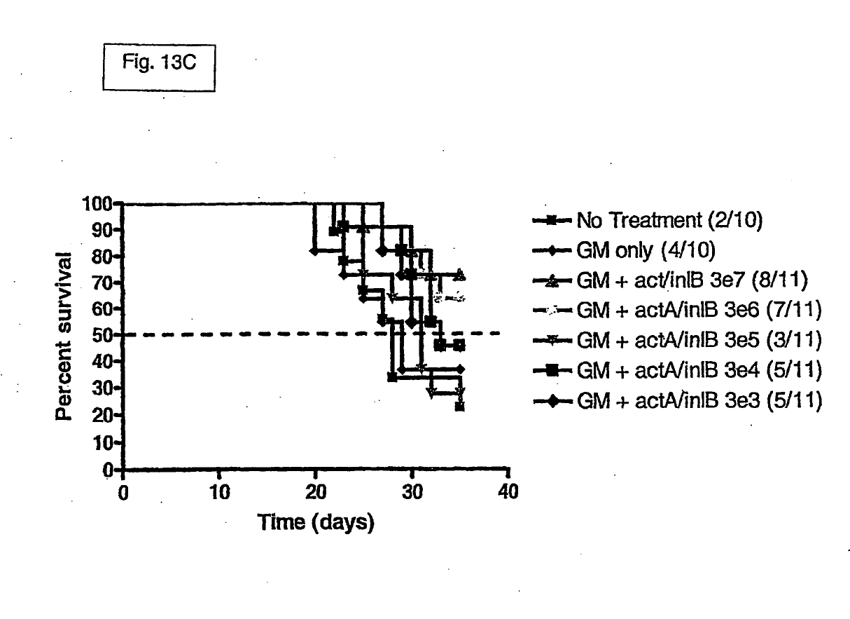

Mice were inoculated with CT26 tumor cells on day zero (FIG. 12). The dose of the CT26 tumor cells used to generate the tumors was 0.1 million cells. Therapeutic treatment was as follows: no treatment (-▪-; filled squares); treatment with GM-CSF vaccine only (-⋄-; open diamonds); treatment with GM-CSF vaccine and cyclophosphamide (CTX) (-Δ-; open triangles); treatment with GM-CSF plus Listeria ΔactA (-•-; filled circles); treatment with GM-CSF, cyclophosphamide, and Listeria ΔactA (-∇-; open inverted triangles); GM-CSF plus Listeria ΔactAΔinlB (-□-; open squares); or treatment with GM-CSF, cyclophosphamide, and Listeria ΔactAΔinlB (-♦-; filled diamonds) (FIG. 13). CTX was given at 100 mg CTX per kg body weight (intraperitoneally; i.p.). Cyclophosphamide was from Sigma (St. Louis, Mo.), and dissolved in HBSS before injecting in animals.

Tumor cells were administered at day zero. For this study, each mouse receiving the GM-CSF vaccine received three doses of the GM-CSF vaccine (at t=3, 15, and 31 days). Where cyclophosphamide was administered, there was only one dose, and it was given at t=day 2. Listeria ΔactA was administered at t=6, 19, and 34 days (1×10⁷ CFU). Listeria ΔactAΔinlB was also administered at the same days, and at the same dosage (t=6, 19, and 34 days (1×10⁷ CFU)) (FIG. 12).

Lowest rates of survival were found in the no treatment group, and in mice receiving GM-CSF vaccine only (FIG. 12). Mice treated with the GM-CSF vaccine plus Listeria ΔactA showed a marked increase in survival time, where about 30% survival was found at t=40 days. The following concerns groups receiving CTX. Where the GM-CSF vaccine was supplemented with CTX only, 90% survival was found at t=45 days. Greater rates of survival were found when the GM-CSF vaccine was supplemented with CTX plus Listeria. For example, when the GM-CSF vaccine was supplemented with CTX plus Listeria ΔactAΔinlB, survival at t=55 days was 100% (-♦-; filled diamonds) (FIG. 13).

The present invention provides a method comprising administering an attenuated Listeria (e.g., L. monocytogenes ΔactA or L. monocytogenes ΔactAΔinlB), with attenuated tumor cells (e.g. irradiated metastatic cells), where the cells had been engineered to express a cytokine, e.g., GM-CSF, with an agent that inhibits action of T regulatory cells (e.g., CTX). In the present invention, the Listeria are not engineered to comprise any nucleic acid encoding any heterologous antigen, e.g., a tumor or infectious agent antigen.

X. Titrating Tumor-Bearing Mice with Listeria, with Constant Administration of Vaccine.

FIGS. 13A to 13C disclose results where various numbers of Listeria were administered to tumor-bearing mice (constant administration of vaccine). In detail, the work involved titrating CT26 cell-tumor bearing mice with Listeria ΔactA (constant GM-CSF vaccine treatment) or with Listeria ΔactAΔinlB (constant GM-CSF vaccine treatment).

In the following studies, tumor-bearing mice were “titrated” with various amounts of attenuated Listeria. In all cases, GM-CSF vaccine was administered on three days (at t=days 3, 17, and 31), and in all cases, Listeria ΔactA (or Listeria ΔactAΔinlB) was administered on three days (at t=days 6, 20, and 34).

Mice were inoculated with CT26 tumor cells. Mice received either no treatment (-▪-; squares); GM-CSF vaccine only (-▴-; triangles); GM-CSF vaccine with 3×10⁷ Listeria (-▾-; inverted triangles); GM-CSF vaccine with 1×10⁷ Listeria (-♦-; diamonds); or GM-CSF vaccine with 3×10⁶ Listeria (-•-; filled circles). FIG. 13A depicts results where the administered attenuated Listeria were deleted in only one virulence gene (Listeria ΔactA) (range of 3×10⁶ to 3×10⁷ bacteria), while FIG. 13B shows results with Listeria deleted in two different virulence genes (Listeria ΔactAΔinlB) (range of 3×10⁶ to 3×10⁷ bacteria). FIG. 13C also depicts results with Listeria ΔactAΔinlB, where the bacteria were administered in the range of 3×10³ to 3×10⁷ bacteria.

Poorest survival rates were found in mice receiving no treatment or administered the GM-CSF vaccine only. Administration of Listeria, along with the GM-CSF vaccine improved survival, where the low and middle bacterial dose levels (3×10³ to 3×10⁵) appeared to provide similar improvement in survivals. Here, the dose of 3×10⁶ bacteria seemed to work as well as 1×10⁷ bacteria. Even better survival was found at the high dose (3×10⁷ bacteria). At the high bacterial dose (with GM-CSF vaccine), about 30-40% survival was found at t=53 days (FIGS. 13A and B).

FIG. 13C demonstrates that the highest survival rate was obtained with the highest level of administered bacteria (3×10⁷ bacteria; -▴-; triangle), where 70% survival was found at t=35 days. Survival was similar, or slightly lower, with administration of 3×10⁶ bacteria (-•-; filled circle). Still lower levels of survival were found with administration with lesser numbers of bacteria (3×10⁵ bacteria; -▾-; inverted triangle) (3×10⁴ bacteria; -▪-; squares) (3×10³ bacteria; -♦-; diamonds). At one of the levels of administered bacteria (3×10⁵ bacteria; -▾-; triangles), survival was found to be somewhat better than the no treatment group, though survival was as low as the “no treatment” group at time periods after t=30 days. Results from the “no treatment” group (-▪-; squares) and GM-CSF vaccine only group (-♦-; diamonds) were as indicated.

The present invention provides a method of administering an attenuated Listeria (e.g., Listeria ΔactA or Listeria ΔactAΔinlB) by way of a plurality of doses, and an attenuated tumor vaccine, by way of a plurality of doses. In one aspect, the attenuated tumor is engineered to contain a nucleic acid encoding a cytokine, e.g., GM-CSF. In another aspect, the attenuated tumor is not engineered to contain a nucleic acid encoding a cytokine.

XI. Listeria (not Containing a Nucleic Acid Encoding a Tumor Antigen) Reduced Tumor Metastases to the Lung.

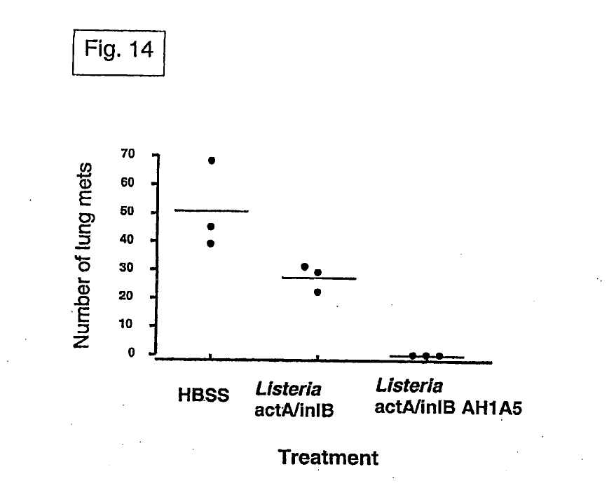

FIG. 14 shows data from lung tumors (not liver tumors). FIG. 14 discloses dose response curves, showing response of lung tumors to various doses of administered Listeria. The tumors arose from CT26 cells injected into the spleen. The figure discloses a control study, where tumor cell-innoculated mice were treated with salt solution (HBSS). Also shown are results from treatment with Listeria ΔactAΔinlB not containing any nucleic acid encoding a tumor antigen (1×10⁷ bacteria administered), and with Listeria ΔactAΔinlB engineered to containing a nucleic acid encoding a positive control tumor antigen (AH1-A5) (1×10⁷ bacteria administered), an epitope derived from gp100. With salt water treatment, there were about fifty lung metastases. With Listeria not engineered to express any tumor antigen, the number of lung metastases was cut in half (about 25-30 lung metastases). With Listeria engineered to express AH1-A5, there were essentially zero lung metastases (FIG. 14).

XII. Listeria (not Engineered to Contain a Nucleic Acid Encoding a Tumor Antigen) Stimulates Long-Term Adaptive Immunity to Tumors.

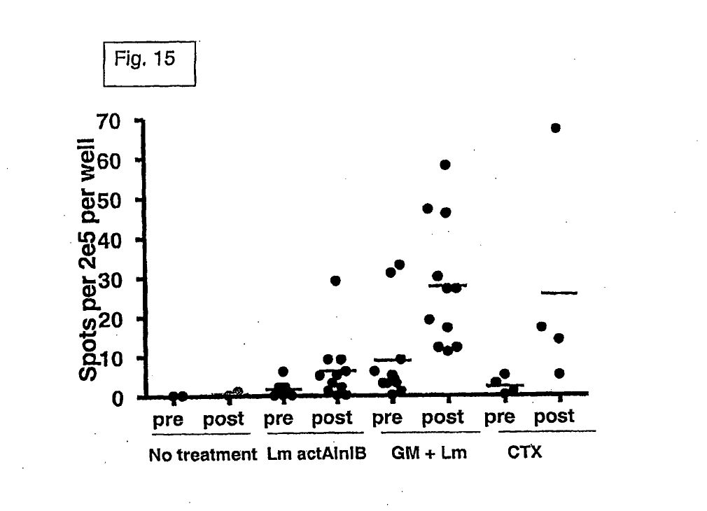

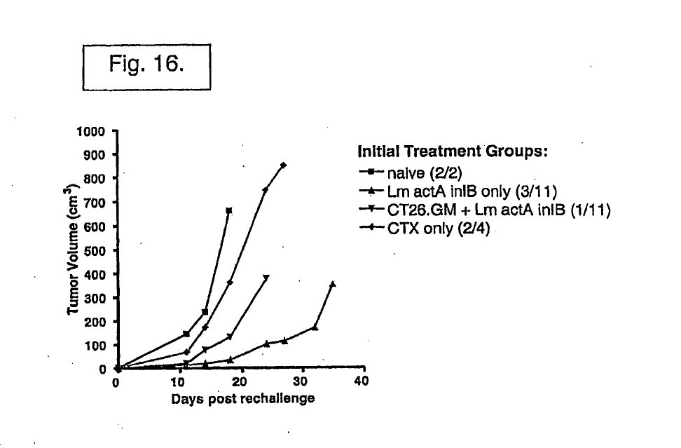

FIGS. 15 and 16 demonstrate that treating tumor-bearing mice with Listeria (Listeria not engineered to encode any heterologous antigen) stimulates adaptive immunity to the tumor, i.e., to antigens of the tumor. Mice were initially inoculated (t=0 days) with CT26 tumor cells by way of the hemispleen model, and then treated with:

(1) No treatment with any therapeutic agent (“naive mice”);

(2) Listeria ΔactAΔinlB (3 cycles of Listeria ΔactAΔinlB beginning at t=3 days after inoculation with the CT26 tumor cells. Administration of Listeria was once weekly for three weeks. The Listeria ΔactAΔinlB had not been engineered to express any tumor antigen;

(3) GM-CSF vaccine with Listeria ΔactAΔinlB (1 injection of Listeria ΔactAΔinlB at t=6 days). Administration of the GM-CSF vaccine was started three days after injecting the tumor cells in the hemispleen, that is, on days 3, 6, and 10; or

(4) Cyclophosphamide (CTX) (50 mg/kg).

At t=100 days (shortly before the re-challenge) and at t=107 days (post re-challenge), surviving mice in each group were assessed for long-term immunity (Elispot assays) to the immunodominant antigen of the CT26 cells (AH1 antigen). The first Elispot assay (pre re-challenge) served as a baseline assay for use in assessing adaptive immune response. The second Elispot assay (107 days; post re-challenge) was used to assess adaptive immune response. At t=102 days, all mice were inoculated with CT26 tumor cells by way of a subcutaneous re-challenge. The subcutaneous CT26 tumor cell re-challenge was with 2×10⁵ cells (twice the dose initially injected in the hemispleen). FIG. 15 demonstrates that the re-challenge with CT26 tumor cells:

(1) Failed to stimulate detectable anti-AH 1-immunity in the group of mice that had never been treated with any therapeutic agent (the “no treatment” group);

(2) Produced a detectable, or modest, Elispot response in the mice that had originally received Listeria ΔactAΔinlB alone;

(3) Produced a stronger Elispot response in mice that had originally received both the GM-CSF vaccine and Listeria ΔactAΔinlB; and

(4) Produced a moderate Elispot response in mice that had originally received only cyclophosphamide (CTX) (FIG. 15).

In short, the results demonstrate that treatment with either Listeria ΔactAΔinlB alone; GM-CSF vaccine and Listeria ΔactAΔinlB; or cyclophosphamide (CTX) alone, can produce a long term effect on the immune system. The long term effect resulted in clearly detectable immune responses to the re-challenge.

Tumor volume was assessed in the days following the CT26 tumor cell re-challenge (FIG. 16). Tumors resulting from the subcutaneous injection presented as bumps under the skin. The dimensions of these tumors were measured topically. The results demonstrated that, in the days following the re-challenge, tumors arising from the re-challenge grew and increased in volume. However, tumor growth was the greatest in the animals that had never received any therapeutic agent, while tumor growth was significantly inhibited in animals that had initially been treated with the Listeria ΔactAΔinlB alone or with GM-CSF vaccine and Listeria ΔactAΔinlB (FIG. 16).

A number of the mice studied in the re-challenge experiment were found to be tumor-free. Regarding these tumor-free mice, the results demonstrated that none of the naive mice (no therapeutic treatment) (out of 2 naive mice in all) were tumor free following the re-challenge; about 50% of the CTX-only mice (out of 4 CTX-only mice in all) were tumor free; while about 75% of the Listeria ΔactAΔinlB only treated mice (out of 11 Listeria ΔactAΔinlB only mice in all) and about 90% of the GM-CSF vaccine plus Listeria ΔactAΔinlB-treated mice (out of 11 GM-CSF vaccine plus Listeria ΔactAΔinlB in all) were tumor free.

The following concerns tumors induced by MC38 cells, rather than CT26 cells. Separate studies with C57BL/6 mice inoculated with MC38 cells demonstrated that all control mice died by t=43 days, with half dying by about t=38 days. Experimental mice administered 3×10⁷ cfu Lm ΔactAΔinlB (doses at t=3, 10, and 17 days), survived to at least t=90 days. In the Lm ΔactAΔinlB-treated group, about half the mice had died by t=50 days, and about 80% had died by t=90 days. The above commentary on MC38 cells refers to a study where CTX was not administered. In short Lm ΔactAΔinlB improved survival to MC38 cells, without any administered CTX. As mentioned earlier, CT26 tumor cells are from Balb/c mice, whereas MC38 tumor cells are from C57Bl/6 mice, where Balb/c mice are Th2 type responders and C57Bl/6 mice are Th1 type responders.

The present invention provides a method comprising administration of a metabolically active Listeria for stimulating adaptive immunity (including long-term adaptive immunity; memory response; and recall response), e.g., to a tumor, cancer, infectious agent, viral, parasitic, or bacterial antigen. The invention encompasses the above method, further comprising administration of one or more of a cytokine, e.g., GM-CSF, an attenuated tumor, an attenuated tumor expressing the cytokine, or an inhibitor of Tregs, such as cyclophosphamide (CTX). In another aspect, the above invention comprises the above method, where the Listeria is not engineered to express a heterologous antigen, e.g., an antigen derived from a tumor cell, cancer cell, or infective agent.

Also provided is a method comprising administering a metabolically active attenuated Listeria for stimulating adaptive immunity (including long-term adaptive immunity; memory response; and recall response), e.g., to a tumor, cancer, infectious agent, viral, parasitic, or bacterial antigen. The invention encompasses the above method, further comprising administration of one or more of a cytokine, e.g., GM-CSF, an attenuated tumor, an attenuated tumor expressing the cytokine, or an inhibitor of Tregs, such as cyclophosphamide (CTX). In another aspect, the above invention comprises the above method, where the Listeria is not engineered to express a heterologous antigen, e.g., an antigen derived from a tumor cell, cancer cell, or infective agent.

XIII. Cytokines.

A. Mouse Cytokines

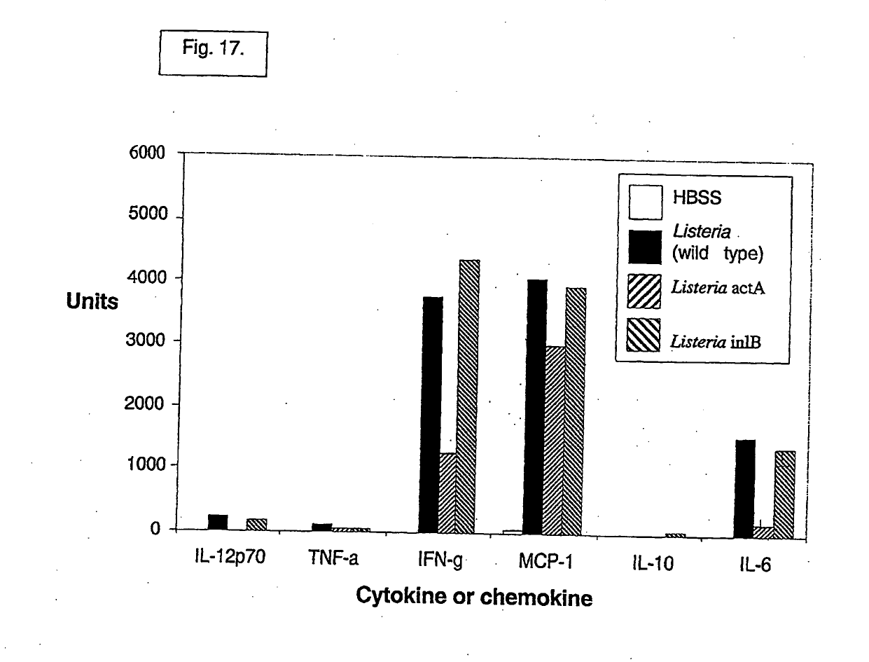

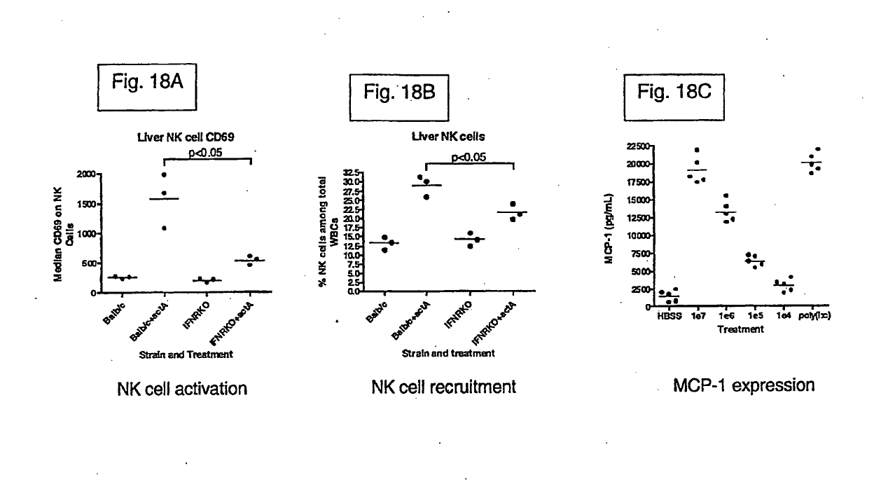

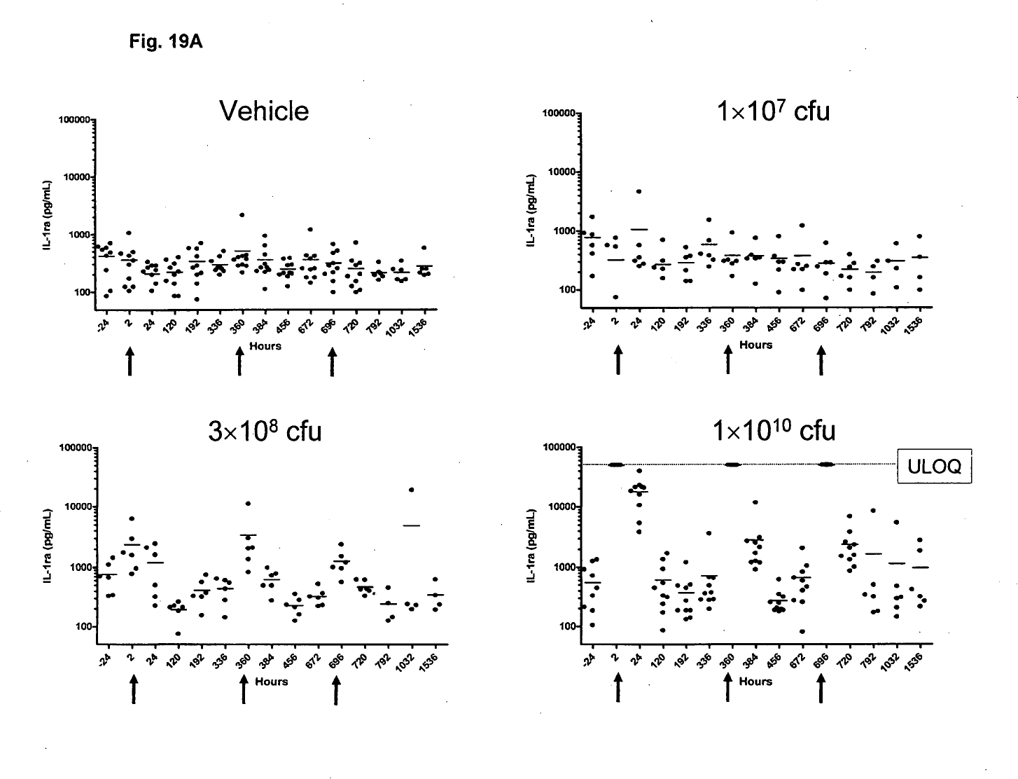

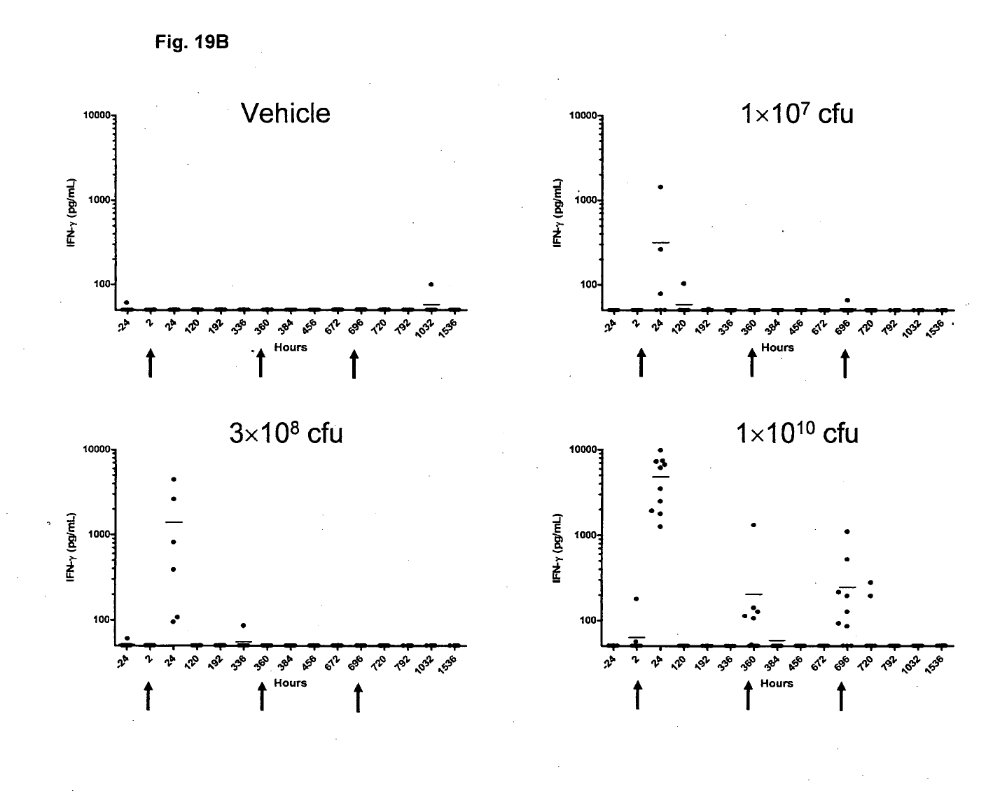

Listeria's influence on cytokine expression in mice is demonstrated in FIG. 17 and FIGS. 18A, 18B, and 18C.

FIG. 17 demonstrates that administering Listeria stimulates the expression of a number of cytokines. Serum cytokine levels are shown, following a single intravenous administration of Listeria. Cohorts of mice (3 per group) were sampled for serum 24 hrs following a single intravenous administration of salt (HBSS), or of 0.1 LD₅₀ L. monocytogenes ΔactA, L. monocytogenes ΔinlB, or wild-type L. monocytogenes. The cytokines assayed were the p70 subunit of interleukin-12 (IL-12); TNFalpha; IFNgamma; MCP-1; IL-10; and IL-6. Cytokine levels were determined using the Cytokine Bead Array (CBA) kit (BD Biosciences, San Jose, Calif.). Results are represented as mean+/−SD. The results demonstrated that wild type Listeria, Listeria ΔactA; and Listeria ΔinlB; stimulated expression of interferon-gamma; MCP-1; and IL-6. Of these three, administering wild type Listeria or Listeria ΔactA resulted in the most marked increases in expression of these cytokines.

The present invention provides a method for stimulating expression of IFN-gamma; MCP-1; IL-6; or both IFN-gamma and MCP-1; both IFN-gamma and IL-6; or both IL-6 and MCP-1; or all three of MCP-1, IL-6, and IFN-gamma, comprising administering Listeria ΔactA; Listeria ΔinlB; or attenuated mutant Listeria ΔactΔinlB.

Also provided is a method for stimulating MCP-1 dependent immune response; IFN-gamma dependent immune response; or IL-6 dependent immune response, comprising administering Listeria ΔactA; Listeria ΔinlB; or attenuated mutant Listeria ΔactΔinlB. Moreover, what is provided is a method for stimulating an immune response dependent on both IFN-gamma and MCP-1; both IFN-gamma and IL-6; both MCP-1 and IL-6; or dependent on all three of IFN-gamma, MCP-1, and IL-6, comprising administering Listeria ΔactA; Listeria ΔinlB; or attenuated mutant Listeria ΔactΔinlB (FIG. 17).

The following concerns FIGS. 18A, 18B, and 18C. Listeria (not engineered to express any heterologous antigen) provoked the activation and recruitment of NK cells to the liver, where these effects were shown to be mediated by interferon-beta. The following demonstrates that IFN-alpha/beta signaling is required for activation and recruitment of NK cells to the liver in response to Listeria. Livers from 3 individual mice per experimental group were harvested 24 hrs. post single IV administration of 1×10⁷ c.f.u. of L. monocytogenes ΔactA. The harvested livers were processed, and the leukocyte population was counted by forward and side scatter with flow cytometry. The NK cell compartment was evaluated by counting cells that stained positive for both DX5 and/or CD69. The results demonstrated that, with Listeria administration, CD69 expression on NK cells increased from a basal level of about 250 (no Listeria) to about 1500 (yes Listeria) (FIG. 17A). This increase was markedly reduced where mice were IFN receptor knockout mice, thus demonstrating a role of interferon-alpha/beta in Listeria's influence on NK cells activation. Regarding NK cell recruitment, FIG. 17B demonstrates that the percent of NK cells among the total hepatic white blood cells increased from about 13% (no Listeria) to about 30% (yes Listeria), where this effect was reduced in the IFN receptor knockout mice.

In addition to assessing NK cell number, serum cytokine was measured, 24 hrs following a single IV administration of L. monocytogenes ΔactA/ΔinlB. Cohorts of five mice were given a single IV administration of L. monocytogenes ΔactAΔinlB at the dose indicated in the figure and serum was sampled 24 hrs later. The positive control for innate activation consisted of a single IV dose of 100 micrograms of poly I:C (FIG. 18C). The results demonstrate the dramatic effect of Listeria in increasing serum MCP-1. In detail, mice were titrated with Listeria ΔactAΔinlB, where the titration involved zero; 10,000; 0.1 million; 1 million; and 10 million administered bacteria. Again, the results demonstrate that Listeria stimulates an increase in MCP-1 expression. Methods for assessing DX5 expression are available (see, e.g., Arase, et al. (2001) J. Immunol. 167:1141-1144).

Cytokine levels were measured in serum, where the serum was from blood harvested from mice at various times after administering Listeria or a toll-like receptor (TLR) agonist. The treatment groups were (1) Salt water (HBSS) treatment only (0.2 ml); (2) L. monocytogenes ΔactAΔinlB (1×10⁷ bacteria); (3) L. monocytogenes Δhly (deleted in the gene encoding listeriolysin) (3×10⁸ bacteria); (4) L. monocytogenes killed but metabolically active (KBMA) (3×10⁸ bacteria) (see, e.g. Brockstedt, et al. (2005) Nat. Medicine 11:853-860); (5) heat killed L. monocytogenes ΔactAΔinlB (3×10⁸ bacteria); (6) poly(I:C) (0.1 mg); or (7) CpG (0.1 mg). Peripheral blood was withdrawn at various times, and assessed for cytokine concentration (Mouse Cytokine/Chemokine LINCOplex® Kit Catalog # MCYTO-70K; Linco, St. Charles, Mo.; or BD® Cytometric Bead Array, San Jose, Calif.). CpG was CpG ODN 1826, purchased through Invivogen. Cytokine levels were measured on samples withdrawn at 2, 4, 8, 12, and 24 hours after administration of bacteria or TLR agonist.

The cytokines measured included granulocyte-colony stimulating factor (G-CSF); interferon-gamma (IFN-gamma); interleukin-1alpha (IL-1alpha); interleukin-6 (IL-6); interleukin-10 (IL-10); interleukin-12p70 (IL-12p70); interleukin-13 (IL-13); IP-10; KC (mouse ortholog of IL-8); MCP-1; MIP-1α; and TNF.

The following cytokines were also measured, where in the case of these cytokines, they were not detected in serum: IL-1beta; IL-2; IL-4; IL-5; IL-7; IL-9; IL-15; IL-17; and granulocyte-monocyte-colony stimulating factor (GM-CSF). In short, these cytokines were not detected under the recited conditions.

Table 7 discloses some of the results.

TABLE 7
Cytokine concentrations in mouse serum after administering Listeria,
poly(I:C), or CpG.

| | | | | | Group 5 | | |
|---|---|---|---|---|---|---|---|
| | | | | | Listeria | | |
| | | | | | ΔactA | | |
| | | Group 2 | Group 3 | Group 4 | ΔinlB | Group 6 | |
| | Group 1 | Listeria | Listeria | Listeria | (heat | Poly | Group 7 |
| | HBSS | ΔactAΔinlB | Δhly | (KBMA) | killed) | (I:C) | CpG |
| | Kinetics and cytokine concentration (pg/ml) | | | | | | |
| G-CSF | Basal | Linear | Early | Early | Early | Early rise | Early rise |
| | level | rise from | high rise | high rise | high rise | to 1200 pg/ml | to 3000 pg/ml |
| | (300-600 pg/ml). | 2-24 h, to | to 18,000 pg/ml | to 25,000-100,000 | to 13,000 pg//ml | (2 h), with | (2 h), with |
| | | a peak of | (2 h), with | pg/ml | (2 h), then | peak at | peak at |
| | | 15,000 pg/ml | peak at | (2-12 h), | gradual | 8-12 h | 8-12 h |
| | | (24 h). | 8-12 h | then | return to | (6,000 pg/ml), | (10,000 pg/ml), |
| | | | (20,000 pg/ml), | return to | basal at | and drop | and drop |
| | | | and | basal | 24 h. | to basal | to basal |
| | | | gradual | (24 h). | | (24 h). | (24 h). |
| | | | drop to | | | | |
| | | | basal | | | | |
| | | | (24 h). | | | | |
| IFN- | Basal | Near | Near | Near | Basal | Early rise | Increase |
| gamma | level | basal at | basal at | basal at | level. | to 15 pg/ml | detected |
| | (<0.05 pg/ml). | 2-4 h, | 2 h, with | 2 h, with | | (2 h) with | at 4 h (10 pg/ml) |
| | | with rise | rise at 4 h, | rise at 4 h, | | plateau | and 8 h |
| | | at 8 h, and | and low | and peak | | (25-30 pg/ml) | (23 pg/ml), |
| | | high | peak (65 pg/ml) | (760 pg/ml) | | at | with |
| | | peak | at | at | | 4-8 h, | decrease. |
| | | (2500 pg/ml) | 8 h, with | 8 h, with | | followed | |
| | | at | decrease. | decrease. | | by return | |
| | | 12 h. | | | | to near | |
| | | | | | | basal. | |
| IL-1alpha | Basal | Near | Early | Early | Early | Near | Early |
| | level (5 pg/ml). | basal at | increase | increase | increase | basal at | increase |
| | | 2 h, with | to 750 pg/ml | to 1200 pg/ml | to 700 pg/ml | 2 h, with | to 130 pg/ml |
| | | linear | (2 h), with | (2 h), with | (2 | plateau | (2 h), with |
| | | increase | peak at | peak at | and 4 h), | (170-300 pg/ml) | peak at |
| | | starting | 4 h (1000 pg/ml) | 8 h (1500 pg/ml) | and | at | 8 h (600 pg/ml) |
| | | from | and drop | and drop | gradual | 8-12 h, | and drop |
| | | 4-24 h to | towards | towards | drop | and basal | to basal |
| | | peak (900 pg/ml) | basal by | basal by | towards | at 24 h. | by 24 h. |
| | | at | 24 h. | 24 h. | basal by | | |
| | | 24 h. | | | 24 h. | | |
| IL-6 | Basal | Basal at | Early rise | Early | Early | Early rise | Early rise |
| | level | 2 h, with | (to 1000 pg/ml) | high rise | peak (750 pg/ml) | (7500 pg/ml) | (2000 pg/ml) |
| | (5-70 pg/ml). | increase | with peak | with peak | (2 h) with | at | at |
| | | starting at | at 2 h, | at 2 h | return to | 2 h, with | 2 h, with |
| | | 4 h, peak | with | (5000 pg/ml), | basal by | gradual | gradual |
| | | at 8 h | gradual | with | 4 h. | drop | drop |
| | | (1250 pg/ml), | return to | gradual | | (5000 pg/ml | (1000 pg/ml |
| | | and drop | basal at | drop | | at | at |
| | | to 200 pg/ml) | 24 h. | (2500 pg/ml | | 4 h) to | 4 h) to |
| | | (24 h). | | at | | near basal | near basal |
| | | | | 4 h) to | | at 12 h. | at 12 h. |
| | | | | basal at | | | |
| | | | | 24 h. | | | |
| IL-10 | Basal | Basal at | Moderate | Moderate | Sporadic | Sporadic | Basal at |
| | level | 2 h, with | levels at | levels at | spikes in | spikes in | 2 h, with a |
| | (<1 pg/ml). | gradual | 2 h, 4 h, | 2 h and | the range | the range | peak at |
| | | increase, | 12 h, with | 12 h, with | of 14-35 pg/ml | of 14-80 pg/ml | 4 h (200 pg/ml), |
| | | starting at | a peak at | a peak at | found at | found at | and low |
| | | 8 h, to a | 8 h (250 pg/ml). | 4-8 h | 2 h and at | 2 h and at | plateau |
| | | peak at | Basal at | (200-250 pg/ml). | 8 h. | 8 h. Basal | from |
| | | 24 h (40 pg/ml). | 24 h. | Basal at | Basal at | at 4 h, | 8-24 h |
| | | | | 24 h. | 4 h, 12 h, | 12 h, 24 h. | (30-60 pg/ml). |
| | | | | | 24 h. | | |
| IL-12p70 | Basal | Basal at | Early rise | Early rise | Early rise | Early rise | Early rise |
| | level | 2 h, with | with a | to | with a | with a | with a |
| | (40-60 pg/ml). | gradual | peak of | 200-400 pg/ml | peak of | peak of | peak of |
| | | increase | 100-150 | found at | 180 at 2 h | 300 at 2 h | 1100 at |
| | | to a peak | at 2 h-8 h, | 2-4 h, | followed | followed | 2 h |
| | | of 550 pg/ml | followed | with a | by a | by a | followed |
| | | (12 h), | by a | peak of | steady | steady | by a |
| | | and | decrease | 1500 pg/ml | decrease | decrease | steady |
| | | decrease | to 65-75 pg/ml | (8 h), and | (basal at | (basal at | decrease, |
| | | to 250 pg/ml | (12-24 h). | drop to | 8-24 h). | 12-24 h). | reaching |
| | | (24 h). | | near basal | | | basal at |
| | | | | levels | | | 24 h. |
| | | | | (12 h-24 h). | | | |
| IL-13 | Basal | Basal | Basal | Early rise | Basal | Basal | Basal |
| | level | levels at | levels at | to about | levels at | levels at | levels at |
| | (3.2 pg/ml). | 2 h-24 h, | 2 h-24 h, | 80 pg/ml | 2 h-24 h, | 2 h-24 h, | 2 h-24 h, |
| | | but with | but with | (2 h), with | but with | but with | but with |
| | | sporadic | sporadic | near basal | sporadic | sporadic | sporadic |
| | | spikes (to | spikes (to | level at | spikes (to | spikes (to | spikes (to |
| | | about 250 pg/ml) | about 250 pg/ml) | 4 h, and | about 250 pg/ml) | about 200 pg/ml) | about 100 pg/ml) |
| | | at | at | peak to | at | at | at |
| | | 24 h in | 8 h in | 380 pg/ml | 12 h in | 4 h, 12 h, | 4 h and 8 h |
| | | some | some | (8 h), and | some | 24 h, in | in some |
| | | mice. | mice. | drop to | mice. | some | mice. |
| | | | | basal | | mice. | |
| | | | | (12 h, | | | |
| | | | | 24 h), | | | |
| | | | | with | | | |
| | | | | sporadic | | | |
| | | | | spikes at | | | |
| | | | | 12 h and | | | |
| | | | | 24 h. | | | |
| IP-10 | Basal | Early rise | Early rise | Early rise | Early rise | Early rise | Early rise |
| | level | at 2 h to | to 900 pg/ml | to 1000 pg/ml | to 820 pg/ml | to 1400 pg/ml | to 1000 pg/ml |
| | (<10 pg/ml). | 500 pg/ml, | (2 h) with | (2 h) with | at | at | at |
| | | with a | a plateau | plateau at | 2 h, with | 2 h, with | 2 h, with |
| | | peak | at this | this level | gradual | peak at | peak at |
| | | occurring | level to | continuing | drop, | 4 h (2100 pg/ml), | 4 h(1400 pg/ml), |
| | | at 8 h-24 h | 8 h, and | to 12 h | with near | and | and |
| | | (1400 pg/ml | decrease | with | basal | gradual | gradual |
| | | at | to 250 pg/ml | slight | levels at | drop (800 pg/ml | drop (400 pg/ml |
| | | 12 h). | (24 h). | drop to | 12 h and | at | at |
| | | | | 700 pg/ml | 24 h. | 24 h). | 24 h). |
| | | | | (24 h). | | | |
| KC | Basal | Early rise | Early | Early | Early rise | Early rise | Early rise |
| | level | to 500 pg/ml | high rise | high rise | to 1500 pg/ml | to 900 pg/ml | to 1100 pg/ml |
| | (<25 pg/ml). | (2 h) with | to 2000 pg/ml | to 5100 pg/ml | at | (2 h) with | (2 h) with |
| | | lower | (2 h) with | at | 2 h, with | near basal | drop to |
| | | levels at | maintained | 2 h, with | return to | levels | 500 pg/ml |
| | | 4 h-8 h | low levels | gradual | a maintained | (4 h-24 h). | (4 h), and |
| | | (200 pg/ml), | (<300 pg/ml) | drop, and | basal | | basal |
| | | increase | at | near basal | level at | | levels at |
| | | at 12 h | 4-12 h, | levels at | 4 h-8 h. | | 12 h-24 h. |
| | | (700 pg/ml) | and basal | 24 h. | | | |
| | | and drop | level at | | | | |
| | | at 24 h | 24 h. | | | | |
| | | (200 pg/ml). | | | | | |
| MCP-1 | Basal | Early rise | Early | Early | Early rise | Early | Early rise |
| | level | by 2 h | high rise | high rise | to 7000 pg/ml | high rise | to 10,000 pg/ml |
| | (100 pg/ml). | (3000 pg/ml) | to 9000 pg/ml | to 16,000 pg/ml, | (2 h), | to 29,000 pg/ml | (2 h), then |
| | | with peak | (2 h), with | with a | followed | (2 h), then | gradual |
| | | at 12 h | gradual | peak at | by sudden | gradual | drop to |
| | | (10,000 pg/ml) | decrease, | 4 h | drop at | drop | 5000 pg/ml |
| | | and maintained | and basal | (24,000 pg/ml), | 4 h, with | (10,000 pg/ml | (8 h) and |
| | | levels at | levels at | and | near basal | at | low levels |
| | | 24 h (5500 pg/ml). | 12 h and | gradual | levels | 8 h), and | (800 pg/ml) |
| | | | 24 h. | drop to | (8 h-24 h). | low levels | at |
| | | | | 1000 pg/ml | | (1000 pg/ml) | 12 h-24 h. |
| | | | | (24 h). | | at | |
| | | | | | | 12 h and | |
| | | | | | | 24 h. | |
| MIP-1a | Basal | Basal | Early rise | Early | Early rise | Early rise | Early rise |
| | level | level until | to 850 pg/ml | high rise | to 600 pg/ml | to 1800 pg/ml | to 1300-1500 |
| | (3.2 pg/ml). | 12 h, | at | to 3000 pg/ml | (2 h), with | (2 h) with | at 2-4 h, |
| | | where | 2 h, | (2 h), | drop to | gradual | with |
| | | level at | followed | with4500 pg/ml | near basal | drop | gradual |
| | | 24 h is | by drop | peak at | by 4 h. | towards | return to |
| | | 280 pg/ml. | to near | 4 h, then | | basal by | near basal |
| | | | basal by | drop. | | 8 h. | at 12 h. |
| | | | 8 h. | | | | |
| TNF | Basal | Slow | Early | Early | Early rise | Early rise | Plateau of |
| | level | increase | peak of | peak of | to 500 pg/ml | to 1500 pg/ml | 300-550 |
| | (3.2 pg/ml). | evident | 550 pg/ml | 1600 pg/ml | (2 h) with | (2 h) with | at 2-8 h, |
| | | by 2-4 h, | at 2 h, | by | return to | return | followed |
| | | with peak | with | 2 h, with | basal by | towards | by drp to |
| | | (250 pg/ml) | gradual | gradual | 8 h. | basal by | basal at |
| | | at | drop to | drop to | | 8 h. | 24 h. |
| | | 12 h. | basal by | basal by | | | |
| | | | 12 h. | 12 h. | | | |
| The present invention, in certain embodiments, provides methods of modulating, e.g., stimulating, expression of one or any combination of G-CSF; IFN-gamma; IL-1alpha; IL-6; IL-10; IL-12p70 (interleukin-12 is a heterodimeric cytokine of p40 and p35 subunits); IL-13; IP-10; KC; MCP-1; MIP-1a; TNF. Provided is a method of stimulating | | | | | | | |
| # or inhibiting a condition or disorder that is dependent on, or is modulated by, one or any combination of G-CSF; IFN-gamma; IL-1alpha; IL-6; IL-10; IL-12p70 (interleukin-12 is a heterodimeric cytokine of p40 and p35 subunits); IL-13; IP-10; KC; MCP-1; MIP-1a; TNF. | | | | | | | |

B. Monkey Cytokines

Cytokine expression was measured in non-human primates that were administered Lm ΔactAΔinlB. Cynomolgus monkeys, both male and female, were administered with vehicle, 1×10⁷, 3×10⁸, or 1×10¹⁰ cfu of Lm ΔactAΔinlB. A total of 32 cynomolgous monkeys (16 per gender) were randomly assigned to the four dose groups.

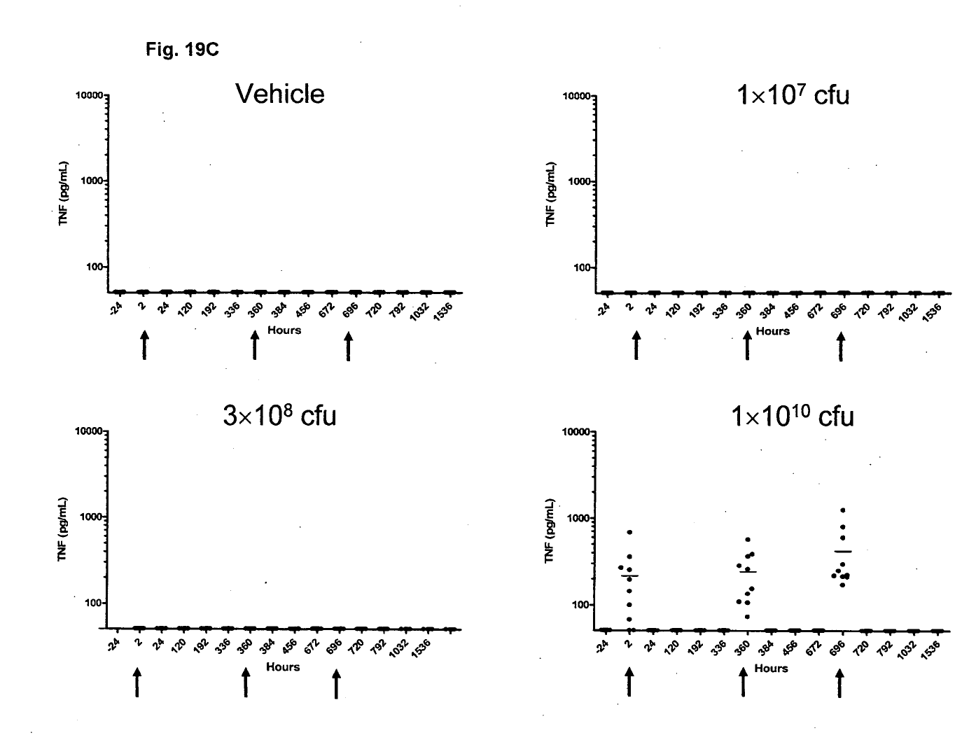

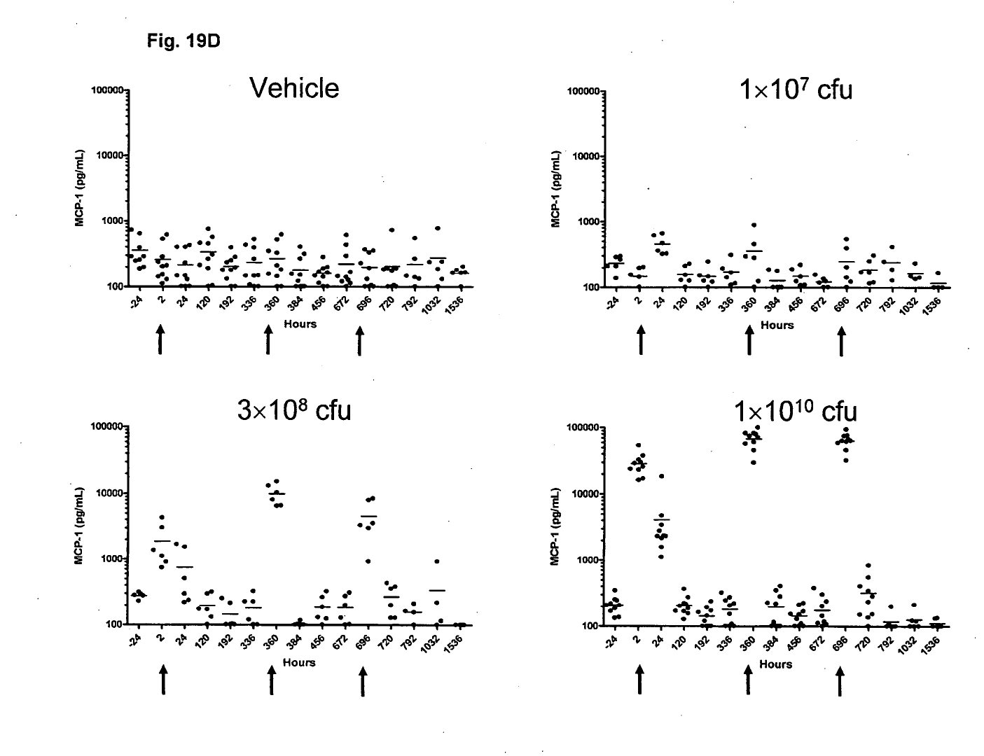

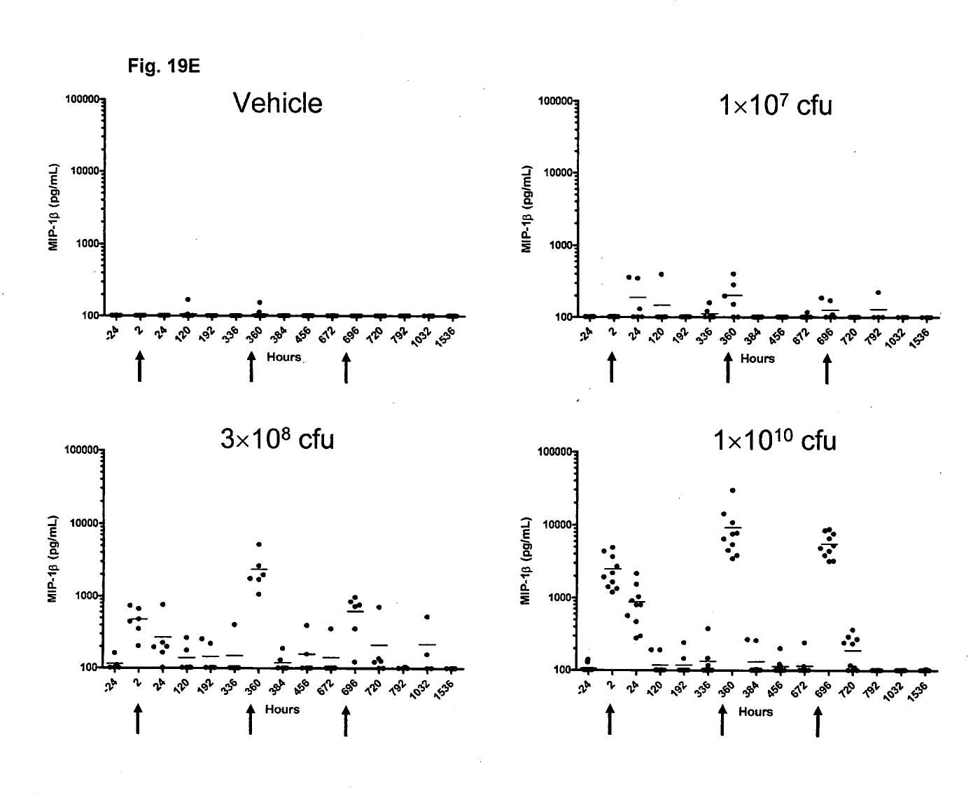

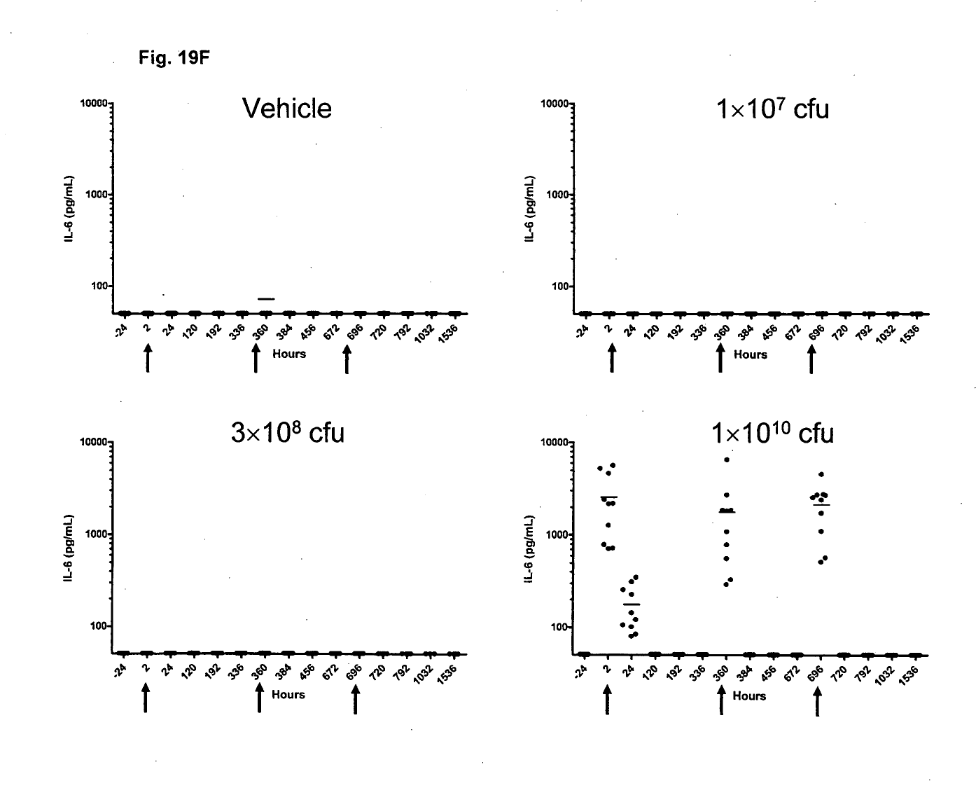

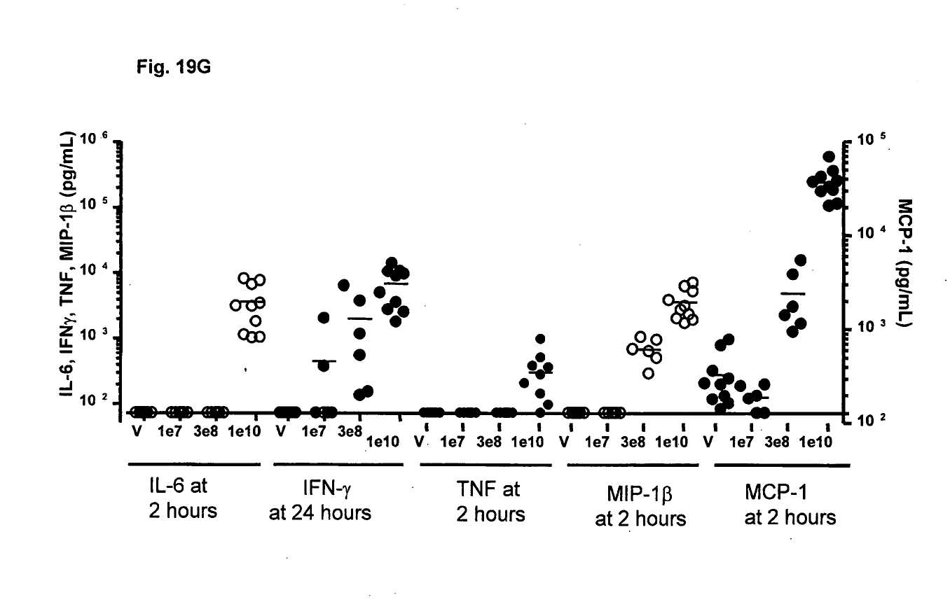

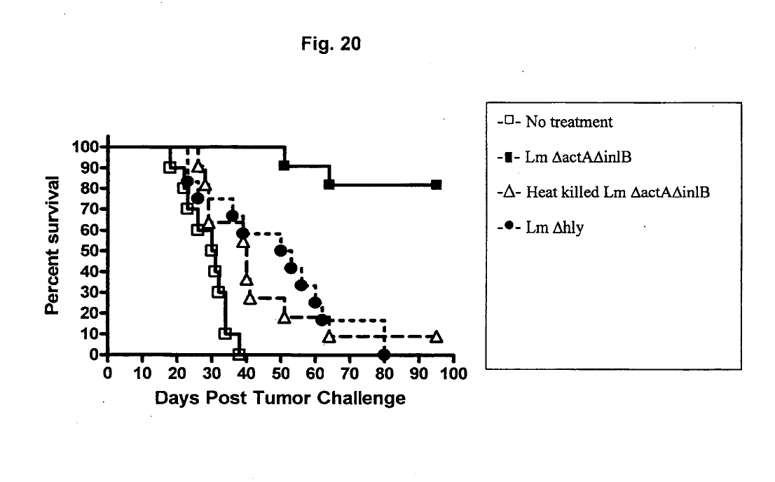

Administration was via a 30 minute (i.v.) infusion every week for five total doses. Serial serum and plasma samples were analyzed for the respective cytokines: IL-1 Ralpha; IFNgamma; TNFalpha; MCP-1; MIP-1beta; and IL-6 (FIGS. 19A-F). FIG. 19G also shows cytokine expression by cynomolgus monkeys, and discloses cytokine expression following the first infusion of Lm ΔactAΔinlB. IL-6, IFNgamma, TNF, MIP-1beta, and MCP-1 were measured after the initial infusion, as indicated (FIG. 19G). Serum levels of each of these cytokines increased, specifically in response to Lm ΔactAΔinlB, where the increases all demonstrated a dependence on the dose.

XIV. Optimal Anti-Tumor Activity Requires Cytosolic Entry by Listeria monocytogenes.

Liver-specific CT-26 metastasis were established following the protocol described by Jain et al., Ann. Surg. Oncol. 10:810-820 (2003) with slight modifications. CT26 is an N-nitroso-N-methylurethane-induced murine colon adenocarcinoma cell line derived from Balb/c mice. Cells were maintained in culture in Dulbecco modified Eagle medium (DMEM) supplemented with 10% fetal bovine serum (FBS) and penicillin/streptomycin (50 U/ml).

On Day 0, female Balb/c mice were implanted with 1×10⁵ CT26 cells via hemispleen surgery. Briefly, Balb/c mice were anesthetized via isoflurane and a left flank incision was made to expose the spleen. The spleen was divided into two hemispleens by using two medium-size Horizon titanium surgical clips (Weck Closure Systems, Research Triangle Park, N.C.) leaving the vascular pedicles intact. Using a 27-gauge needle, 10⁵ viable CT-26 cells were injected into one half of the spleen. The CT-26 tumor cells then flow into the splenic and portal veins and deposit in the liver. The vascular pedicle draining the cancer-contaminated hemispleen was ligated and the CT-26-contaminated hemispleen was excised, leaving a functional hemispleen free of tumor cells.

To understand the necessity for bacterial entry into the cytosol, tumor bearing mice were immunized with either live Lm ΔactAΔinlB, heat-killed (HK) Lm ΔactAΔinlB, or L. monocytogenes unable to produce LLO (Δhly, unable to escape the phagocytic vacuole). The Listeria were diluted in HBSS to the appropriate concentration and administered intravenously into the mice in a final volume of 100 or 200 μl. Balb/c mice bearing 3 day established hepatic metastasis were treated with Lm ΔactAΔinlB (3e7 cfu), heat-killed Lm ΔactAΔinlB (3e8 cfu), or Δhly Lm (3e8 cfu). The vaccinations were given on day 3, 10, and 17. The percent survival is shown in FIG. 20 for each group (n=6-10 mice per group).

Both HK-Lm ΔactAΔinlB and LLO-deficient L. monocytogenes significantly prolonged the median survival (MST 40 and 52 days respectively) relative to untreated controls (MST 31 days), although a majority of the animals succumbed to tumor burden. This is in striking contrast to mice that were treated with Lm ΔactAΔinlB where 80% of Lm ΔactAΔinlB treated mice remained tumor free for the duration of the study (FIG. 20). These results indicate that optimal Lm-induced anti-tumor activity requires cytosolic entry.

Many modifications and variations of this invention, as will be apparent to one of ordinary skill in the art, can be made to adapt to a particular situation, material, composition of matter, process, process step or steps, to preserve the objective, spirit, and scope of the invention. All such modifications are intended to be within the scope of the claims appended hereto without departing from the spirit and scope of the invention. The specific embodiments described herein are offered by way of example only, and the invention is to be limited by the terms of the appended claims, along with the full scope of the equivalents to which such claims are entitled; and the invention is not to be limited by the specific embodiments that have been presented herein by way of example.

What is claimed is:

1. A method for treating a mammal having a cancerous or non-listerial infectious condition, wherein the cancerous or infection condition is in the liver of the mammal, comprising administering to the mammal an effective amount of a metabolically active, attenuated Listeria, wherein the Listeria does not comprise a nucleic acid encoding a non-listerial antigen capable of stimulating a specific immune response against the condition, and wherein the attenuated Listeria is administered to the mammal in multiple doses.

2. The method of claim 1, wherein the cancerous or infectious condition is inhibited or reduced in the mammal by the administration of the effective amount of the attenuated Listeria.

3. The method of claim 1, wherein survival of the mammal is enhanced by the administration of the effective amount of the attenuated Listeria.

4. The method of claim 1, wherein the attenuated Listeria is attenuated in one or more of:
a. growth;
b. cell to cell spread;
c. binding to or entry into a host cell;
d. replication; or
e. DNA repair.

5. The method of claim 4, wherein the attenuated Listeria is attenuated in:
a. cell to cell spread; or
b. both cell-to-cell spread and entry into nonphagocytic cells.

6. The method of claim 1, wherein the Listeria is attenuated by one or more of:
a. an actA mutation;
b. an inlB mutation;
c. a uvrA mutation;
d. a uvrB mutation;
e. a uvrC mutation;
f. a nucleic acid targeting compound; or
g. a uvrAB mutation and a nucleic acid targeting compound.

7. The method of claim 6, wherein the Listeria is attenuated by:
a. an actA mutation; or
b. both an actA mutation and an inlB mutation.

8. The method of claim 7, wherein the nucleic acid targeting compound is a psoralen.

9. The method of claim 1, wherein the Listeria cannot do one or more of:
a. form colonies;
b. replicate; or
c. divide.

10. The method of claim 1, wherein the Listeria is killed, but metabolically active (KBMA).

11. The method of claim 1, wherein the attenuated Listeria is administered intravenously.

12. The method of claim 1, wherein the attenuated Listeria is administered in three or more doses.

13. The method of claim 1, wherein the attenuated Listeria is one or both of:
a. not administered orally to the mammal, or
b. administered as a composition that is at least 99% free of other types of bacteria.

14. The method of claim 1, wherein the attenuated Listeria is administered to the mammal in a pharmaceutical composition.

15. The method of claim 1, wherein the mammal has not previously been administered a vaccine against the cancerous or infectious condition.

16. The method of claim 1, wherein the method does not further comprise administering a vaccine against the cancerous or infectious condition to the mammal.

17. The method of claim 1, wherein the mammal comprises the cancerous condition.

18. The method of claim 17, wherein the condition comprises a tumor or cancer.

19. The method of claim 18, wherein the condition comprises a cancer that has metastasized to the liver.

20. The method of claim 19, wherein the cancer is colorectal cancer.

21. The method of claim 1, wherein the mammal comprises the non-listerial infection.

22. The method of claim 1, wherein the infectious condition comprises one or more of:
a. hepatitis B;
b. hepatitis C;
c. human immunodeficiency virus (HIV);
d. cytomegalovirus (CMV);
e. Epstein Barr virus (EBV); or
f. leishmaniasis.

23. The method of claim 1, wherein the administering stimulates an innate immune response against the condition.

24. The method of claim 1, wherein the administering stimulates an acquired immune response against the condition.

25. The method of claim 1, wherein the administering stimulates one, or any combination, of a:
a. NK cell;
b. NKT cell;
c. dendritic cell (DC);
d. monocyte or macrophage;
e. neutrophil; or
f. toll like receptor (TLR) or nucleotide binding oligomerization domain (NOD) protein,
as compared with immune response in the absence of the administering of the effective amount of the attenuated Listeria.

26. The method of claim 1, wherein the administering stimulates increased expression of any one, or any combination, of:
a. CD69;
b. interferon-gamma (IFNgamma);
c. interferon alpha (IFNalpha) or interferon beta (IFNbeta);
d. interleukin 12 (IL 12);
e. monocyte chemoattractant protein (MCP 1); or
f. interleukin 6 (IL 6),
as compared with expression in the absence of the administering of the effective amount of the attenuated Listeria.

27. The method of claim 1, wherein the mammal is human.

28. The method of claim 1, wherein the Listeria is Listeria monocytogenes.

29. The method of claim 1, further comprising administering one, or any combination of:
a. an agonist or antagonist of a cytokine;
b. an inhibitor of a T regulatory cell (Treg); or
c. a tumor cell attenuated in growth or replication.

30. The method of claim 29, wherein the inhibitor of a Treg is cyclophosphamide (CTX).

31. The method of claim 1, wherein the effective amount comprises at least about 1×10³ CFU/kg or at least about 1×10³ Listeria cells/kg.

32. A method for inducing an immune response against a cancer cell, tumor, or non-listerial infective agent in a mammal, wherein the mammal comprises the cancer cell, tumor, or non-listerial infective agent in its liver, comprising administering to the mammal an effective amount of a metabolically active, attenuated Listeria, wherein the Listeria does not comprise a nucleic acid encoding a non-listerial antigen capable of stimulating a specific immune response against the condition, wherein the attenuated Listeria is administered to the mammal in multiple doses, and wherein the attenuated Listeria is one or both of:
a. not administered orally to the mammal, or
b. administered as a composition that is at least 99% free of other types of bacteria.

33. The method of claim 32, wherein the attenuated Listeria is attenuated in one or more of:
a. growth;
b. cell to cell spread;
c. binding to or entry into a host cell;
d. replication; or
e. DNA repair.

34. The method of claim 33, wherein the attenuated Listeria is attenuated in:
a. cell to cell spread; or
b. both cell-to-cell spread and entry into nonphagocytic cells.

35. The method of claim 32, wherein the Listeria is attenuated by one or more of:
a. an actA mutation;
b. an inlB mutation;
c. a uvrA mutation;
d. a uvrB mutation;
e. a uvrC mutation;
f. a nucleic acid targeting compound; or
g. a uvrAB mutation and a nucleic acid targeting compound.

36. The method of claim 35, wherein the Listeria is attenuated by:
a. an actA mutation; or
b. both an actA mutation and an inlB mutation.

37. The method of claim 35, wherein the nucleic acid targeting compound is a psoralen.

38. The method of claim 32, wherein the Listeria cannot do one or more of:
a. form colonies;
b. replicate; or
c. divide.

39. The method of claim 32, wherein the Listeria is killed, but metabolically active (KBMA).

40. The method of claim 32, wherein the attenuated Listeria is administered intravenously.

41. The method of claim 32, wherein the attenuated Listeria is administered in three or more doses.

42. The method of claim 32, wherein the mammal is not administered a vaccine capable of stimulating a specific immune response against the cancer cell, tumor, or non-listerial infective agent.

43. The method of claim 32, wherein the mammal comprises the cancer cell or tumor.

44. The method of claim 32, wherein the mammal comprises the non-listerial infective agent in its liver.

45. The method of claim 32, wherein the immune response inhibits or reduces one, or any combination, of the:
a. number or tumors or cancer cells;
b. tumor mass; or
c. titer of an infectious agent,
in the mammal.

46. The method of claim 32, wherein the administering stimulates an innate immune response against the cancer cell, tumor, or non-listerial infective agent.

47. The method of claim 32, wherein the administering stimulates an acquired immune response against the cancer cell, tumor, or non-listerial infective agent.

48. The method of claim 32, wherein the administering stimulates one, or any combination, of a:
a. NK cell;
b. NKT cell;
c. dendritic cell (DC);
d. monocyte or macrophage;
e. neutrophil; or
f. toll like receptor (TLR) or nucleotide binding oligomerization domain (NOD) protein,
as compared with immune response in the absence of the administering of the effective amount of the attenuated Listeria.

49. The method of claim 32, wherein the administering stimulates increased expression of any one, or any combination, of:
a. CD69;
b. interferon-gamma (IFNgamma);
c. interferon alpha (IFNalpha) or interferon beta (IFNbeta);
d. interleukin 12 (IL 12);
e. monocyte chemoattractant protein (MCP 1); or
f. interleukin 6 (IL 6),
as compared with expression in the absence of the administering of the effective amount of the attenuated Listeria.

50. The method of claim 32, wherein the mammal is human.

51. The method of claim 32, wherein the Listeria is Listeria monocytogenes.

52. The method of claim 32, wherein the immune response comprises stimulating one or both of:
a. an increase in the percent of hepatic leukocytes that is NK cells, compared to the percent without the administering of the attenuated Listeria; or
b. an increase in expression of an activation marker by a hepatic NK cell, compared to the expression without the administering of the attenuated Listeria.

53. The method of claim 32, wherein the effective amount of attenuated Listeria comprises at least about 1×10³ CFU/kg or at least about 1×10³ Listeria cells/kg.

54. A method for inducing an immune response against a cancer cell, tumor, or non-listerial infective agent in a mammal, wherein the mammal comprises the cancer cell, tumor, or non-listerial infective agent in its liver, comprising administering to the mammal an effective amount of a metabolically active, attenuated Listeria, wherein the Listeria does not comprise a nucleic acid encoding a non-listerial antigen capable of stimulating a specific immune response against the condition, wherein the attenuated Listeria is administered to the mammal in multiple doses, and wherein the attenuated Listeria is one or both of:
a. administered in a pharmaceutical composition; or
b. a non-naturally occurring strain.

55. A method for treating a mammal having a cancerous or non-listerial infectious condition, wherein the cancerous or infectious condition is in the liver of the mammal, comprising administering to the mammal an effective amount of a metabolically active, attenuated Listeria, wherein the Listeria does not comprise a nucleic acid encoding a non-listerial antigen capable of stimulating a specific immune response against the condition, and wherein the Listeria is administered to the mammal in the absence of a separately generated, vaccine-induced immune response to the cancerous or infectious condition in the mammal.
